(19) United States
(12) Patent Application Publication
(10) Pub. No.: US 2026/0115202 A1
(43) Pub. Date: Apr. 30, 2026

(54) TOPICAL COMPOSITIONS COMPRISING AN ESTETROL COMPONENT AND USE OF SAID COMPOSITIONS FOR WOUND HEALING

(71) Applicant: NEURALIS SA, Liège (BE)

(72) Inventors: Héloise GAIDE CHEVRONNAY, Liège (BE); Céline GERARD, Liège (BE); Lali RONSONI ZANCAN, Liège (BE); Matthew James HARDMAN, Liège (BE)

(73) Assignee: NEURALIS SA, Liège (BE)

(21) Appl. No.: 19/118,510

(22) PCT Filed: Oct. 6, 2023
(86) PCT No.: PCT/EP2023/077760

§ 371 Date: Apr. 4, 2025

(30) Foreign Application Priority Data
Oct. 7, 2022 (EP) 22200360.0

(51) Int. Cl. A61K 31/565; A61K 9/00; A61K 9/06; A61K 47/10; A61K 47/32; A61K 47/34; A61K 47/38; A61P 17/02

(52) U.S. Cl. CPC A61K 31/565; A61K 9/0014; A61K 9/06; A61K 47/10; A61K 47/32; A61K 47/34; A61K 47/38; A61P 17/02

(57) ABSTRACT

The present invention relates to topical compositions comprising an estetrol component and the use of said compositions to deliver an effective amount of an estetrol component to the skin, particularly for wound healing. Optionally, the estetrol component may be comprised in a composition that further comprises ingredients that are favourable for wound healing. The invention relates in particular to compositions such as estetrol-comprising gels and in particular hydrogels, creams, and ointments. The compositions of the present invention have a beneficial effect on the wound healing process and patient recovery.

### FIELD OF THE INVENTION

The present invention broadly relates to treating wounds of the skin by means of an effective amount of an estetrol component. Furthermore, the present invention relates to pharmaceutical compositions comprising an estetrol component suited for treating wounds of the skin. The pharmaceutical compositions and related methods subject of the invention have favourable effects on wounds, the wound healing process, and ultimately patient recovery.

### BACKGROUND OF THE INVENTION

Throughout a lifetime, each individual is exposed numerous times to adverse stimuli or events that result in cutaneous wounds, such as injury, surgery, burns, and pathology-induced wounds of skin and optionally underlying tissues. Often, these wounds heal without a substantial burden for the subject following routine adequate care, which includes practices such as disinfection and/or temporarily covering the wound site. However, complications may arise if the individual has impaired wound healing, if there is excessive inflammation or infection at the wound site, or in situations wherein the wound site covers a substantial area of the individual.

While often perceived as a “basic” functionality of skin tissue, wound healing is the culmination of different complex molecular mechanisms. In normal skin tissue (i.e., non-wounded skin tissue), a protective layer against external stimuli is formed by the epidermis (outmost skin layer) and dermis (corium) (Kolarsick et al., JDNA. 2011). Upon damage introduced to said protective layer, a repair process is initiated that aims to repair the damage. This repair process has been described in the art in detail and comprises the subsequent steps of hacmostasis (blood clotting), inflammation, proliferation (growth of new tissue), and maturation (tissue remodelling).

When examined in more detail, the above repair process is characterised by a number of molecular mechanisms that also have been the subject of study in the art (e.g. in Rodrigues et al., Physiol Rev. 2019). In brief, during the clotting phase (commonly considered a part of the inflammation phase), a fibrin clot is generated to prevent further blood loss. The inflammation phase is characterised by initial vasoconstriction and subsequent vasodilation, and recruitment of cells such as neutrophils, monocytes and macrophages. The proliferation phase is characterised by angiogenesis, fibroblast differentiation, and formation of granulation tissue. The granulation tissue formation allows for re-epithelialisation by epithelial cells (keratinocytes) which migrate to cover the wound site. Additionally, the later part of the proliferation phase encompasses fibroblast-mediated contraction of the wound. The final wound healing phase, i.e. the maturation phase, is characterised by the rearrangement and cross-linking of collagen fibres formed at the wound site that increase the tensile strength of the wound and induce formation of scar tissue. In individuals that have impaired wound healing, one or more of the above phases or sub-processes thereof are disturbed, or essentially lacking (Avishai et al., EPMA J. 2017).

The importance of proper wound care cannot be overstated, particularly because the prevalence of chronic wounds is expected to increase in the coming decades as a consequence of increasing prevalence of chronic diseases such as diabetes, cancer, and autoimmune diseases. Epidemiological studies have already warned of this burgeoning healthcare burden. A marked increase has been reported over a 10-year period, with a prevalence of 605,036,000 in 2015 compared to 492,883,000 in 2005 based on global, regional and national data from over 195 countries and territories (reported by the Global Burden of Disease (GBD) 2015 Disease and Injury Incidence and Prevalence Collaborators, Lancet, 2016). Global estimates suggest that at least 7 million people suffer complications following surgery each year, including at least 1 million deaths. Delayed acute wound healing increases the post-surgical risk of morbidity and mortality. A good example are surgical site infections, which are the second leading cause of hospital-acquired infections. Major complications in acute wounds are linked to age-related failure to heal and hormone deficiency. Here, the 65+ population (currently 15-28% of total) is increasing globally.

While currently numerous wound care strategies exist (both mechanical and pharmaceutical), there remains an unmet need for effective wound care strategies and pharmaceutical compositions that aid in said wound care. Such compositions would ideally aid the wound healing of subjects with impaired wound healing, such as elderly subjects and subjects affected by chronic wounds caused by for example a lack of motility and/or underlying pathologies.

### SUMMARY OF THE INVENTION

As evidenced in detail by the examples enclosed herewith, the present inventors have surprisingly found that estetrol is particularly suited for use in wound healing, including wound healing in subjects characterised by impaired wound healing. By extensive experimentation, it was found that estetrol components can be safely included in pharmaceutical compositions aiming to assist in wound healing. Said compositions have no or limited effect on the increase in uterine weight that can occur with topical use of estrogens in female subjects through inadvertent systemic exposure. More particularly, the inventors have found that inclusion of particular amounts of an estetrol component in pharmaceutical compositions aiming to assist in wound healing provides an unprecedented compromise between efficacy and adverse effects such as an increase in uterus weight. The absence of uterus weight increase indicates that systemic effects of the pharmaceutical compositions comprising an estetrol component are limited and arguably even absent at certain dosages, even upon inclusion of a permeation enhancer.

From the in vitro and in vivo wound-healing experiments follows that estetrol exerts at least similar effects as estradiol on e.g. wound closure, re-epithelialisation, and anti-inflammation. Estradiol and estetrol treatment increases fibroblast ER expression, while estetrol also promotes fibronectin expression and inhibits MMP activity. Estetrol also seems to promote dermal fibroblast migration in scratch assays and promote epidermal keratinocyte migration to a higher extent than estradiol. Further, estetrol is show to modulate wound-relevant epidermal gene expression with a slightly higher magnitude of effect than estradiol and to promote wound closure more efficiently than estradiol. These improvements on wound-healing combined with reduced systemic effects (e.g. on uterine weight gain) make estetrol compositions an attractive alternative for estradiol and other estrogen-based compositions used for wound-healing.

Finally, in some embodiments, the compositions are additionally characterised by a gradual release profile which is a further advantage in the context of wound healing. The present invention thus contributes significantly to new and innovative wound care strategies.

Accordingly, in a first aspect the invention is directed to a pharmaceutical composition comprising of from 0.02% to 1.5% (w/w) of an estetrol component, preferably comprising of from 0.05% to 1.2% (w/w) of an estetrol component, of from about 0.02% to about 1% (w/w) of an estetrol component of from about 0.03% to about 1% (w/w) of an estetrol component, preferably of from about 0.04% to about 1% (w/w) of an estetrol component, more preferably of from about 0.05% to about 1% (w/w) of an estetrol component, most preferably from about 0.06% to about 0.5% (w/w) of an estetrol component, more preferably of from about 0.09% to about 1.1% (w/w), even more from 0.1% to 1% (w/w) of an estetrol component, most preferably of from 0.3% to 0.7% (w/w) of an estetrol component. Preferably, said composition is for topical use, or for topical application, such as for topical application to the skin.

Preferably, said composition does not result in a significant systemic effect in the subject upon or after topical application.

In particular embodiments, the pharmaceutical composition comprises of from about 0.03% to about 0.12% (w/w) of an estetrol component, preferably of from about 0.04% to about 0.08% (w/w) of an estetrol component, more preferably of from about 0.05% to about 0.07% w/w of an estetrol component, most preferably about 0.06% (w/w) of an estetrol component.

In particular embodiments, the pharmaceutical composition further comprises a permeation enhancer that enables permeation through the stratum corneum and/or permeation through the wound eschar.

In particular embodiments, the pharmaceutical composition is a composition for use in topical wound healing.

In another aspect, the invention is directed to a (pharmaceutical) composition comprising an estetrol component for use in topical wound healing, wherein the composition optionally further comprises a permeation enhancer enabling permeation through the stratum corneum.

In particular embodiments, the (pharmaceutical) composition comprises of from about 0.01% to about 5% (w/w) of an estetrol component, preferably of from about 0.02% to about 1% (w/w) of an estetrol component, more preferably of from about 0.03% to about 0.75% (w/w) of an estetrol component, yet more preferably of from about 0.04% to about 0.5% (w/w) of an estetrol component, most preferably about 0.06% (w/w) of an estetrol component.

In yet another aspect, the invention is directed to a hydrogel formulation comprising of from about 0.02% to about 1.5% (w/w) of an estetrol component, more particularly of about 0.05 to 1.2% (w/w), even more particularly of from about 0.09% to about 1.1% (w/w), or from about 0.1% to about 1% (w/w).

In particular embodiments, the hydrogel comprises from about 0.05 to about 1.3% (w/w), more particularly from about 0.08 to about 1.2% (w/w), even more particularly of from about 0.09% to about 1.1% (w/w), or from about 0.1% to about 1% (w/w) of an estetrol component. In alternative embodiments, the hydrogel comprises from about 0.03% to about 0.75% (w/w) of an estetrol component, preferably of from about 0.04% to about 0.5% (w/w) of an estetrol component, more preferably of from about 0.05% to about 0.25% (w/w) of an estetrol component, most preferably about 0.06% (w/w) of an estetrol component.

As can be derived from the examples section, a 0.06% (w/w) estetrol component was shown to have no effect on uterus weight increase in the tested mice, which is in strong contrast to the effect on uterus weight increase by applying the commercially available Estrogel®; comprising 0.06% estradiol.

In addition, also higher concentrations of 0.22 and 0.5% (w/w) estetrol still had a significantly lower effect on uterus weight increase in mice as compared to Estrogel®. This shows that topical application of a composition comprising estetrol is less prone to result in systemic effect in the subject, which is particularly important in female subjects. In particular embodiments, the hydrogel further comprises a permeation enhancer that enables permeation through the stratum corneum.

In particular embodiments, the hydrogel is for use in topical wound healing.

Optionally, the (pharmaceutical) composition of any of the aspects and embodiments described herein is in the form of a formulation selected from the group consisting of: emulsions, suspensions, ointments, pastes, lotions, gels (including hydrogels), foams, mousses, and creams.

In embodiments wherein the (pharmaceutical) composition is a hydrogel, said hydrogel is characterised by a favourable release profile, e.g. more favourable when compared to other formulations such as but not limited to creams.

In a preferred embodiment, the hydrogel described herein is characterised by a mean cumulative amount of estetrol component released of at least about 2.5 μg/cm², at least about 5 μg/cm², at least about 7 μg/cm², at least about 10 μg/cm², at least about 15 μg/cm², at least about 20 μg/cm², at least about 25 μg/cm², preferably at least about 50 μg/cm², more preferably at least 100 μg/cm², more preferably at least about 150 μg/cm², more preferably at least about 200 μg/cm² within the square root of 1 hour across an isopore membrane in a receptor solution of 40:30:30 v/v/v ethanol:PEG400:water. Depending on the surface of the wound, the concentration of the estetrol component may need to be reduced in order not to result in a cumulative administration of estetrol in too high amounts. This can be easily calculated by a physician or pharmacist.

In preferred embodiments, the hydrogel described herein is characterised by a mean cumulative amount of estetrol component released of at least about 50 μg/cm², at least about 100 μg/cm², at least about 150 μg/cm², at least about 200 μg/cm², at least about 250 μg/cm² at least about 300 μg/cm², at least about 350 μg/cm², at least about 400 μg/cm², at least about 450 μg/cm² within 8 hours across an isopore membrane in a receptor solution of 40:30:30 v/v/v ethanol:PEG400:water. More preferably, the hydrogel described herein is characterised by a mean cumulative amount of estetrol component released of at least about 80 μg/cm².

Preferably, the hydrogel described herein is characterised by a mean % applied dose of the estetrol component into a receptor solution of 40:30:30 v/v/v ethanol:PEG400:water of at least about 15%, preferably at least about 20% after 1 hour and/or a mean % applied dose of the estetrol component into a receptor solution of 40:30:30 v/v/v ethanol:PEG400:water of at least about 40%, preferably at least about 50%, more preferably at least 80% after 8 hours and most preferably at least 90% after 8 hours.

In any of the above aspects, the (pharmaceutical) composition, (pharmaceutical) composition for use, or hydrogel may comprise a permeation enhancer in an amount of from about 0.5% to about 60% (w/w), or preferably in an amount of from about 0.05% to about 5% (w/w). Preferably, the permeation enhancer comprises a substance or molecule that enables permeation through the stratum corneum (i.e. a permeation enhancing molecule), and a solvent. Preferably, the permeation enhancing molecule is selected from the group consisting of: ethanol, ether such as diethylene glycol monoethyl ether (Transcutol®), benzyl alcohol, fatty acids and esters thereof, or any combination thereof. Preferably, the permeation enhancer comprises a solvent comprising one or more polyethylene glycols (PEG), propylene glycol (PG), or combinations thereof. Preferably, the PEG is a PEG having a molecular weight of between about 200 g/mol and about 600 g/mol such as a PEG selected from the group consisting of: PEG200, PEG300, PEG400, PEG500, PEG600, or any combination thereof. More preferably, the permeation enhancer comprises a solvent comprising a PEG having a molecular weight of about 400 g/mol such as PEG400. In some aspects, the permeation enhancer comprises up to 50% PEG400) (w/w) and/or from about 15% to 45% PEG400) (w/w), and/or from about 20% to 40% PEG400) (w/w), and/or from about 30% to 35% PEG400) (w/w). Most preferably, the permeation enhancer comprises of from about 14% to about 21% PEG400) (w/w) and/or from about 10% to about 25% PEG (w/w). In alternative preferred embodiments, the permeation enhancer comprises from about 0.5% to about 10% of permeation enhancer such as for example PEG400. In further alternative preferred embodiments, the permeation enhancer comprises from about 0.5% to about 5% of permeation enhancer such as for example PEG400.

In certain embodiments, the (pharmaceutical) composition, (pharmaceutical) composition for use, or hydrogel comprises benzyl alcohol, preferably in an amount of from about 1% to about 3%.

In any of the above aspects, the (pharmaceutical) composition, (pharmaceutical) composition for use, or hydrogel may comprise a thickener. In particular embodiments, the (pharmaceutical) composition, (pharmaceutical) composition for use, or hydrogel comprises a thickener in an amount of from about 0.3% to about 20% (w/w), or preferably in an amount of from about 0.3% to about 3% (w/w), or more preferably in an amount of from 0.5% to 3% (w/w). Preferably, the (pharmaceutical) composition, (pharmaceutical) composition for use, or hydrogel comprises a thickener selected from the group consisting of a hydroxyethyl cellulose (HEC), a carboxymethyl cellulose (CMC), a high molecular weight cross-linked acrylic based polymer, a non-ionic triblock copolymer, or any combination thereof. Preferably, the high molecular weight cross-linked acrylic based polymer is Carbopol®.

Preferably, the HEC is HEC250 HHX. Preferably, the non-ionic triblock copolymer has an approximate molecular mass of between about 1800 and about 4000 and a polyoxyethylene content of from about 70 to about 80%. Preferably, the non-ionic triblock copolymer is selected from a poloxamer, such as poloxamer 188, poloxamer 407, or a combination thereof.

In any of the above aspects, the (pharmaceutical) composition, (pharmaceutical) composition for use, or hydrogel may comprise a preservative. In particular embodiments, the (pharmaceutical) composition, (pharmaceutical) composition for use, or hydrogel may comprise a preservative in an amount of from about 1% to about 10% (w/w), preferably of from about 1% to about 3% (w/w). Preferably, the preservative is selected from the group consisting of: lysozyme, nisin, quaternary ammonium preservatives, parabens, phenoxyethanol, benzyl alcohol, chlorobutanol, phenol, sorbic acid, thimerosal, natural preservatives, and any combination thereof.

In any of the above aspects, the (pharmaceutical) composition, (pharmaceutical) composition for use, or hydrogel may comprise an emollient. In particular embodiments, the (pharmaceutical) composition, (pharmaceutical) composition for use, or hydrogel may comprise an emollient in an amount of from about 2.5% to about 30% (w/w), preferably of from about 8% to about 12% (w/w), most preferably in an amount of about 10% (w/w). Preferably, the emollient is selected from the group consisting of: glycerol, acetyl alcohol, stearyl alcohol, stearic acid, isopropyl palmitate, squalene, lanolin, glycerin, petrolatum, petroleum, and any combination thereof.

Therefore, in any of the aspects and embodiments described herein, the (pharmaceutical) composition. (pharmaceutical) composition for use, or hydrogel may comprise in addition to the estetrol component a permeation enhancer, a thickener, and as optional ingredient a preservative and/or an emollient, which are each preferably selected from the groups described herein. In any of the aspects and embodiments described herein, the (pharmaceutical) composition may be complemented to 100% (w/w) by means of an aqueous solution such as water.

In particular embodiments, the (pharmaceutical) composition, (pharmaceutical) composition for use, or hydrogel may comprise in addition to the estetrol component (w/w):
from about 0.1% to about 60% (w/w) of a permeation enhancer, preferably wherein said permeation enhancer comprises a permeation enhancer molecule and a solvent or solvent system:
from about 0.3% to about 20% (w/w) of a thickener;
optionally a preservative and/or an emollient;
water up to 100% (w/w).

In further embodiments, the (pharmaceutical) composition, (pharmaceutical) composition for use, or hydrogel may comprise from 0.05% to 0.6% (w/w) estetrol and in addition:
from about 0.1% to about 5% (w/w) of a permeation enhancer, preferably wherein said permeation enhancer comprises a permeation enhancer molecule and a solvent or solvent system:
from about 0.3% to about 3% (w/w) of a thickener.
optionally a preservative and/or an emollient:
water up to 100% (w/w).

In further particular embodiments the (pharmaceutical) composition, (pharmaceutical) composition for use, or hydrogel may comprise in addition to the estetrol component (w/w):
from about 16% to about 20% (w/w) PEG400;
from about 18% to about 22% (w/w) PG:
from about 8% to about 12% (w/w) glycerol:
from about 1% to about 2% (w/w) HEC; and
from about 1.5% to about 2.5% (w/w) benzyl alcohol.

In alternative embodiments the (pharmaceutical) composition, (pharmaceutical) composition for use, or hydrogel may comprise in addition to the estetrol component:
from about 18% to about 22% (w/w) PEG400;
from about 0.1% to about 1% (w/w) Carbopol®; and
from about 4% to about 6% (w/w) Transcutol®.

In yet alternative embodiments the (pharmaceutical) composition, (pharmaceutical) composition for use, or hydrogel may comprise in addition to the estetrol component:
from about 25% to about 55% (w/w) PEG400, preferably from about 35% to about 45% (w/w) PEG400;
from about 0.1% to about 1% (w/w) Carbopol®, preferably from about 0.25% to about 0.75% (w/w) Carbopol®; and
from about 0.1% to about 5% (w/w) Transcutol®, preferably from about 0.75% to about 3% Transcutol®.

In yet alternative embodiments the (pharmaceutical) composition, (pharmaceutical) composition for use, or hydrogel may comprise in addition to the estetrol component:
from about 38% to about +5% (w/w) PEG400;
from about 0.1% to about 1% (w/w) Carbopol®; and
from about 0.8% to about 3% (w/w) Transcutol®.

Aspects of the invention encompass each of the (pharmaceutical) compositions, hydrogels, and alternatives described herein as a medicament, i.e. in a therapeutic and/or prophylactic context. In particular embodiments, the (pharmaceutical) compositions and hydrogels of the above aspects are for use in wound healing. In further embodiments, the use in wound healing corresponds to their use as topical formulations in the treatment of wounds. Similarly, aspects of the invention relate to the use of a pharmaceutical composition or hydrogel described herein for the manufacture of a medicament for the topical treatment of wounds. Related to the preceding, the aspects of the invention equally encompass methods of topical wound treatment comprising administration of any one of the pharmaceutical compositions or hydrogels described herein to a wound or wound site of a subject.

In particular embodiments, the (pharmaceutical) compositions and hydrogels of the above aspects are for use in treatment of acute wounds. Optionally, the acute wound is a surgical wound or a wound caused by acute injury. The wound can also be a partial thickness wound (e.g., at a skin graft donor site). In alternative embodiments, the pharmaceutical compositions and hydrogels of the above aspects are for use in treatment of chronic wounds. Optionally, the chronic wound is a wound caused and/or maintained by a diabetic disease. Other major causative factors of chronic wounds are ischemia, radiation, foreign bodies and prolonged external pressure. Generally chronic wounds are divided into infected or ischemic wound.

In particular embodiments, the medical use or treatment as described herein results in improved histological healing parameters versus non-treated wounds. Preferably, the medical use or treatment results in improved incidence of complete wound closure, accelerated wound closure, and/or facilitation of surgical wound closure.

In preferred embodiments, the medical use or treatment improves the quality of healing also referred to as cosmesis. Particularly for surgical wounds less scaring is observed.

In particular embodiments, the pharmaceutical compositions and hydrogels as described herein show a beneficial effect on inflamed wounds. With the use, the inflammation itself and/or the inflammatory progression is prevented. Wounds can also be prevented from recurring.

In particular embodiments, the (pharmaceutical) compositions and hydrogels of the above aspects are for use in treatment of wounds of subjects having impaired wound healing including an impaired delayed cutaneous wound healing or bacterially delayed wound healing. In particular embodiments, the impaired wound healing is characterized by reduced wound edge migration. In other embodiments, the impaired wound healing is characterized by increased wound edge proliferation.

In particular embodiments, the (pharmaceutical) compositions and hydrogels of the above aspects are for use in treatment of infected wound sites, combat wounds, burns, and chronic leg ulcers. In some embodiments, said infected wounds can be associated with reduced re-epithelialisation, increased proliferation, a heightened inflammatory response and perturbed wound matrix deposition. One particularly studied pathogen in this respect is Klebsiella pneumoniae and the estetrol component containing compositions disclosed herein are shown to reduce inflammation in a model of Klebsiella pneumoniae infected wounds.

In particular embodiments, the (pharmaceutical) compositions and hydrogels of the above aspects are for use in improving re-epithelialization of a wound site, increased proliferation of cells at a wound site, reducing an inflammatory response at/in a wound site, improving matrix deposition like matrix remodeling, and improving angiogenesis at wound sites.

In particular embodiments, the medical use or treatment as described herein results in improved quality of healing, also referred to as cosmesis. This is forming an interesting aspect, particularly in the context of surgical wound healing.

In particular embodiments, the medical use or treatment as described herein results in an improved inflammatory profile of the wound site versus non-treated wounds. Preferably, the medical use or treatment results in an improved macrophage and neutrophil profile indicative for a reduced local wound inflammation versus non-treated wounds.

Optionally, the subject is a subject of elderly age, such as a subject of ≥50 years, or preferably ≥60 years. Optionally, the (pharmaceutical) composition or hydrogel as described herein is applied to the wound site on at least two distinct instances as part of the treatment or medical use. Optionally, the treatment may span a period of at least 1 week, or even 1 month or longer, e.g. 12 weeks. Alternatively, the (pharmaceutical) composition or hydrogel as described herein is applied to the wound site continuously over a prolonged period of time. Preferably, the prolonged period of time corresponds to at least 1 day, at least 1 week, or at least 1 month. In particular embodiments, the (pharmaceutical) composition or hydrogel as described herein is comprised in a wound dressing, bandage, patch, or plaster. Each type of scaffold or matrix could be used. Treatment might also be needed for prolonged durations, e.g. 12 weeks and the forms as described herein are particularly suited to facilitate extended administration periods.

Further aspects of the invention are directed to packaging units comprising the pharmaceutical composition of hydrogel described in any embodiment herein. A packaging unit contains preferably one or more dosage units of the (pharmaceutical) composition (or optionally hydrogel) described herein. Suitable packaging units include any container that is capable of enclosing and preserving liquids. Optionally, the packaging unit is a box, a display unit, an ampoule, a bottle, a vial, a tube, a syringe, a cartridge, a bag, a sachet, a pouch, a film, a laminate, a foil, a can, a cylinder, or a pressurized container.

The above and further aspects and embodiments of the invention are described in the following sections and in the appended claims. The subject matter of the appended claims is hereby specifically incorporated in this specification.

### BRIEF DESCRIPTION OF THE FIGURES

FIG. 1. Mean cumulative amount of estetrol monohydrate released per unit area (μg/cm²) between the 1 and 8 h experimental period (presented as a square root of time) from 10 formulations across an isopore membrane in receptor solution 40:30:30 v/v/v ethanol:PEG400:water. Error bars represent the standard deviation of the mean (n=6).

FIG. 2. Mean cumulative amount of estetrol monohydrate released per unit area (μg/cm²) between the 1 and 8 h experimental period (presented as a square root of time) from 7 aqueous gel formulations across an isopore membrane in receptor solution 40:30:30 v/v/v ethanol:PEG400:water. Error bars represent the standard deviation of the mean (n=6).

FIG. 3. Mean cumulative amount of estetrol monohydrate released per unit area (μg/cm²) between the 1 and 8 h experimental period (presented as a square root of time) from 3 cream formulations across an isopore membrane in receptor solution 40:30:30 v/v/v ethanol:PEG400:water. Error bars represent the standard deviation of the mean (n=6).

FIG. 4. Mean percentage (%) applied dose of estetrol monohydrate released in to the receptor solution between the 1 and 8 h experimental period (presented as a square root of time) from +aqueous gel formulations across an isopore membrane in receptor solution 40:30:30 v/v/v ethanol:PEG400:water. Error bars represent the standard deviation of the mean (n=5-6).

FIG. 5. Mean cumulative amount of estetrol monohydrate released per unit area (μg/cm²) between the 1 and 8 h experimental period (presented as a square root of time) from 4 aqueous gel formulations across an isopore membrane in receptor solution 40:30:30 v/v/v ethanol:PEG400:water. Error bars represent the standard deviation of the mean (n=5-6).

FIG. 6. Effect of topical placebo (PBO), EstroGel® (EG), AG24, AG25 and AG26 application on uterine weight and morphology. Eight-weeks-old female mice were injected subcutaneously with LPS 24 h and 2 h prior to wounding (6 mice per group), EstroGel®, AG24, AG25, AG26 or placebo were applied in a thin layer over the top of the wound one day before, at the time of wounding and at 1, 2, 3 and 4 days after the wounding. Uterine morphological changes at 5 days were assessed by measuring uterine weight (A) and taking uterine photos (B). Scale bar=10 mm. Results are presented as mean+s.e.m. Differences versus placebo were determined using paired t-test: *P-values≤0.05, and ***P-values≤0.001

FIG. 7. Topical E4 treatment promotes re-epithelialization in LPS-induced delayed wound healing mice model. Eight-weeks-old female mice were injected subcutaneously with LPS 24 h and 2 h prior to wounding (6 mice per group). EstroGel®, AG24, AG25, AG26 or placebo were then applied in a thin layer over the top of the wound one day before, at the time of wounding and at 1, 2, 3 and 4 days after the wounding, Histological images were used to calculate wound re-epithelialisation percentage at day 5. Histological sections were subjected to K14 immunohistochemistry to visualize the newly forming epidermis. The degree of re-epithelialization was determined as the length of neo-epidermis divided by the distance between the wound margins, multiplied by 100. Results are presented as mean±s.e.m. Differences versus placebo were determined using paired t-test: *P-values≤0.05, and ***P-values≤0.001, n=6 mice per group. Mean+SEM.

FIG. 8. Topical E4 treatment leads to reduced numbers of wound neutrophils. Immunohistochemistry and quantification of wound neutrophils (A), with representative images (B) for each treatment group. **p≤0.01, n=6 mice per group. Mean+SEM.

FIG. 9. Topical E4 treatment leads to reduced numbers of wound macrophages. Immunohistochemistry and quantification of wound macrophages (A), with representative images (B) for each treatment group. *p≤0.05, **p≤0.01, n=6 mice per group. Mean+SEM.

FIG. 10. Topical E4 treatment promotes a pro-resolution wound phenotype, with reduced M1 marker expression and increased M2 marker expression. Wound tissue RNA was isolated and wound expression of M1 (TNF-α and IL1-β) and M2 (Fizz1 and Ym1) markers quantified via qPCR. *p≤0.05, **p<0.01, n=6 mice per group. Mean+SEM.

FIG. 11. In vitro polarisation of peritoneal macrophages isolated from experimental mice indicates that topical treatments confer systemic effects on immune cell phenotype. Peritoneal macrophages were isolated upon completion of the in vivo study. Cells were cultured and polarised in vitro to M1 or M2 phenotype, with M1 (Tnf-α and iNOS) and M2 (Arg1 and Ym1) marker expression quantified via qPCR. *p≤0.05, n=3 replicates on pooled cells from n=6 mice. Mean+SEM.

FIG. 12. Effect of topical placebo (PBO). EstroGel® (EG). AG26 and AG28 application on uterine weight and morphology. Eight-weeks-old female mice were injected subcutaneously with LPS 24 h and 2 h prior to wounding (6 mice per group). On day 0 only (single administration), or on days −1, 0, 1 and 2 (repeated administration). EstroGel®, AG26, AG28 or their placebos were applied in a thin layer over the top of the wound. Uterine morphological changes at 3 days were assessed by measuring uterine weight. Results are presented as mean±s.e.m. Differences versus placebo were determined using paired t-test: *P-values≤0.05, and ***P-values≤0.001

FIG. 13. A) Effect of topical placebo (PBO). EstroGel® (EG). AG26 and AG28 application on re-epithelialisation. Eight-weeks-old female mice were injected subcutaneously with LPS 24 h and 2 h prior to wounding (6 mice per group). On day 0 only (single administration), or on days −1, 0, 1 and 2 (repeated administration), EstroGel®, AG26, AG28 or their placebos were then applied in a thin layer over the top of the wound. Histological images were used to calculate wound re-epithelialisation percentage at day 3. Histological sections were subjected to K14 immunohistochemistry to visualize the newly forming epidermis. The degree of re-epithelialization was determined as the length of neo-epidermis divided by the distance between the wound margins, multiplied by 100). Results are presented as mean #s.e.m. Differences versus placebo were determined using paired t-test: *P-values≤0.05, and ***P-values≤0.001, n=6 mice per group. Mean+SEM. B) and C) Single topical E4 administration already leads to reduced numbers of wound neutrophils in LPS-induced delayed wound healing mice model. Eight-weeks-old female mice were injected subcutaneously with LPS 24 h and 2 h prior to wounding (6 mice per group). On day 0 only (single administration), or on days −1, 0, 1 and 2 (repeated administration), EstroGel®; (EG, 0.06% E2 gel). AG24 (0.5% E4 gel), AG25 (0.22% E4 gel) and AG26 (0.06% E4 gel) or placebo (PBO) were then applied in a thin layer over the top of the wound. Number of neutrophils cell per cm2 were quantified on histological sections at day 3. **p<0.01, n=6 mice per group. B) Mean+SEM and C) Individual Data Points.

FIGS. 14. E2 and E4 both promote migration of human dermal fibroblasts. (A) Percentage closure was calculated after 36 hours, following treatment with vehicle. E2 or E4 across a range of concentrations. Data from four independent fibroblast donors (n=8). (B) Representative images from the same donor. **p≤0.01, *p≤0.05. Mean+SEM.

FIG. 15. Evaluating whether media/serum composition influences E2 and E4 effects on migration of human dermal fibroblasts. Cells were maintained in DMEM containing 10% charcoal stripped (CS) FBS and then switched to either 2% CS-FBS. 0) % FBS or 2% FBS, scratched and treated with vehicle. E2 or E4. Percentage closure was calculated after 24 hours. Data (A) and representative images (B) from a single donor (n=3). Mean+SEM.

FIG. 16. Both E2 and E4 promote migration of high passage mouse dermal fibroblasts. Percentage closure was calculated after 24 hours, following treatment with vehicle. E2 or E4 across a range of concentrations. Data from cells isolated from a single mouse (n=4). Representative images from cells isolated a single mouse. **p≤0.001, *p≤0.05. Mean+SEM.

FIG. 17. The effects of E4 on mouse dermal fibroblast migration are greater in high passage cells. Percentage closure was calculated after 24 hours, following treatment with vehicle. E2 or E4. Data from low passage cells (a, b) and high passage cells (c, d). Results are presented with cells isolated from 3 independent mice (n=12). Representative images from cells isolated from the same mouse. **p≤0.001, *p≤0.05. Mean+SEM.

FIG. 18. E4 increases expression of both ERα and ERβ in mouse dermal fibroblast (MDFs). Expression of ERα (Esr1). ERβ (Esr2), was measured by qPCR using RNA isolated from MDFs treated with E2. E4, or ERα (PTT) and ERβ (DPN) agonists at 10⁻⁷M. Data from cells derived from n=3 independent mice. **p≤0.01, *p≤0.05. Mean+SEM.

FIGS. 19. E2 and E4 inhibit MMP2 activity in supernatants from human dermal fibroblasts (HDFs). HDFs from n=3 independent donors were treated with E2 or E4. Zymography was performed including standards for MMP2 and MMP9. Data (A) from n=6 experiments with n=3 independent donors. A representative zymogram (B) is shown from a single experiment/donor. **p≤0.01, *p≤0.05. Mean+SEM.

FIG. 20. E4 treatment increases the expression of ECM components in dermal fibroblasts from diabetic, but not wild-type mice. Expression of Collal and Fn1 was measured by qPCR using RNA isolated from MDFs derived from diabetic (db/db) or wild-type mice and treated with E2. E4. Data from cells derived from n=3 independent mice. *p≤0.05. Mean+SEM.

FIG. 21. Both E2 and E4 promote migration of normal human epidermal keratinocytes (NHEK). Percentage closure was calculated after 24 hours, following treatment with vehicle. E2 or E4 across a range of concentrations. Data (A: 15% human keratinocyte growth supplement (HKGS). B: 30% HKGS) from a single primary NHEK donor (n=9-15). Representative images area (C) from a single experiment. **p≤0.01, *p≤0.05. Mean+SEM.

FIG. 22. E4 treated primary mouse epidermal keratinocytes display a strong trend towards increased expression of both ERα and ERβ, and changes in markers of keratinocyte phenotype. Expression of ERα (Esr1), ERβ (Esr2), epithelial to mesenchymal transition marker Snail and keratinocyte differentiation marker Krt1 was measured by qPCR using RNA isolated from MEKs treated with E2 or E4 across a range of concentrations, or E2/E4+ the ER antagonist ICI for 24 hours. Data from cells derived from n=3 independent mice. *p≤0.05. Mean+SEM.

FIG. 23. Experiments in the human immune THP1 cell line reveal anti-inflammatory activity of both E2 and E4. THP1 cells were differentiated to a macrophage phenotype by PMA treatment followed by polarisation to an M1 or M2 phenotype. Expression of M1 marker TNF-α and M2 marker CCL17 was then measured by qPCR using RNA isolated from polarized cells treated with E2 or E4 at different concentrations, n=3. Mean+SEM.

FIG. 24. Pro-inflammatory markers are reduced following treatment with E2 or E4 in M1-stimulated. L929 differentiated, mouse bone marrow derived macrophages (BMDM). MBDMs were differentiated using 20% L929 growth media, and polarised to M1 phenotype using 20 ng/ml IFN-γ. 10 pg/ml LPS for 6 or 24 hours. Co-treatment with E2 or E4 led to a strong trend towards reduced expression of the M1 markers iNOS. Tnf-α and IL1-β (measured via qPCR) using RNA isolated from the treated cells. Data from pooled cells derived from n=3 independent mice. Mean+SEM.

FIG. 25. Pro-inflammatory markers are reduced following treatment with E2 or E4 in M1-stimulated. MCSF differentiated, mouse bone marrow derived macrophages (BMDM). BMDMs were differentiated using 30 ng/ml MCSF, and polarised to M1 using 100 ng/ml IFN-γ, 10 pg/ml LPS for 6 hours. Cells were then co-treated with E2 or E4 at 10⁻⁷M. Expression of the M1 markers iNOS, Tnf-α and IL1-β was measured by qPCR using RNA isolated from the treated cells. Data from pooled cells derived from n=3 independent mice. *p≤0.05. Mean+SEM.

FIG. 26. Pro-inflammatory markers are reduced following treatment with E2 or E4 in M1-polarised murine peritoneal macrophages. Peritoneal macrophages were freshly isolated and immediately pre-treated with E2 or E4 at 10⁻⁷M followed by polarisation to pro-inflammatory M1 phenotype using 100 ng/ml IFN-γ, and 10 pg/ml LPS for 6 hours. Expression of the M1 markers iNOS, Tnf-α and IL1-β was measured by qPCR using RNA isolated from the treated cells. Data from cells derived from n=3 independent mice. **p≤0.01 *p<0.05. Mean+SEM.

FIG. 27. HDF co-treatment with ER-specific antagonists suggests that both ERα and ERβ are involved in E4-promoted fibroblast migration. Percentage closure was calculated after 24 hours, following treatment with vehicle. E2. E4 or E2/E4 and the ERα-specific antagonist MPP or the ERβ-specific antagonist PHTPP. Data from two independent fibroblast donors (n=6). *p≤0.05 vs E4. Mean+SEM.

FIG. 28. Mouse bone marrow derived macrophages (BMDM) co-treatment with ER-specific antagonists suggests that both ERα and ERβ are involved in E4-promoted anti-inflammatory activity. BMDMs were isolated, differentiated using 20% L929 GM, and polarised to pro-inflammatory M1 phenotype using 100 ng/ml IFN-γ and 10 pg/ml LPS for 6 hours. E2 and E4 (10⁻⁷M) stimulated M1 polarised cells were co-treated with the ERα-specific antagonist MPP or the ERβ-specific antagonist PHTPP. Expression of pro-inflammatory marker IL1-β was measured by qPCR using RNA isolated from the treated cells. Cells from n=3 independent mice. **p<0.01 vs E4. Mean+SEM.

FIG. 29. BMDM co-treatment with ER-specific antagonists suggests that both ERα and ERβ are involved in E4-promoted anti-inflammatory activity. BMDMs were differentiated using 20% L929 GM, polarised to M1 using 100 ng/ml IFN-γ. 10 pg/ml LPS for 6. M1 polarised cells were treated with E2. E4. Estrogel® (EG). AG23 placebo or AG23 active formulation, iNOS was measured via qPCR. Cells from n=5-6 independent mice. *p≤0.05, **p≤0.01. Mean+SEM.

### DETAILED DESCRIPTION

As used herein, the singular forms “a”. “an”, and “the” include both singular and plural referents unless the context clearly dictates otherwise.

The terms “comprising”. “comprises” and “comprised of” as used herein are synonymous with “including”. “includes” or “containing”. “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression. The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g. any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined. For example, embodiments directed to products are also applicable to corresponding features of methods and uses.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”. “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, alternative combinations of claimed embodiments are encompassed, as would be understood by those in the art.

Including an estrogen in a pharmaceutical composition suited for topical application has been described in the art. An example of a commercially available topical formulation is EstroGel®, which is an estradiol-comprising gel. EstroGel® is indicated in the treatment of moderate to severe vasomotor symptoms and moderate to severe symptoms of vulvar and vaginal atrophy due to menopause. Although EstroGel® has potential benefits in wound healing, it has not been marketed for this indication as such. Furthermore, it is known that conventional estrogen formulations are suboptimal for direct application to individuals and are characterised by a considerable list of adverse effects that may be linked to the use of estradiol and/or unintended systemic exposure. Such adverse effects can include nausea, vomiting, stomach cramps, bloating, swelling, weight gain, breast pain, breast tenderness, headache, vaginal itching, vaginal discharge, aberrant menstrual regulation, spotting, hair loss, etc. (cf. Mayo Clinic report on transdermal estradiol: Estradiol (Transdermal Route) Side Effects-Mayo Clinic). Hence, known pharmaceutical compositions are accompanied by considerable amounts of unwanted systemic effects on the individual. As described in the summary of the present disclosure, pharmaceutical compositions comprising an estetrol component are provided that have at least similar wound healing properties as pharmaceutical compositions that have been described in the art but display markedly diminished systemic effects. For example, while known estradiol-comprising wound healing compositions lead to a distinct increase in uterus weight indicating a systemic response to estradiol, compositions comprising an estetrol component increase uterus weight to a lesser extent with some compositions even showing virtually no effect on uterus weight while still being effective in wound healing. This observation even remains valid upon inclusion of a permeation enhancer. This allows for the formulation of a pharmaceutical composition comprising a relatively low estrogen dosage. The wound healing properties of such low doses of an estetrol component is additionally remarkable given that estetrol has been historically considered a weak estrogen when compared to other estrogens such as estradiol (Gérard et al., J Endocrinol, 2015). Hence, both the compositions as such as described herein and the medical use for wound healing of estetrol components cannot be anticipated nor envisaged based on what is known in the art.

In view hereof, a first aspect of the invention is directed to a pharmaceutical composition comprising of from about 0.02% to about 0.18 (w/w) of an estetrol component.

The term “estetrol component”, as used throughout this document, encompasses substances selected from the group consisting of estetrol, esters of estetrol, esters of estetrol wherein the hydrogen atom of at least one of the hydroxyl groups has been substituted by an acyl radical of a hydrocarbon carboxylic, sulfonic acid or sulfamic acid of 1-25 carbon atoms, estetrol hydrates such as estetrol monohydrate; and combinations thereof. It is understood that when “estetrol” is mentioned throughout any section of this specification, any estetrol-containing component (i.e. compound) and/or estetrol derivative (such as the ones mentioned above) is also envisaged. More preferably, in the context of the present disclosure, a particularly preferred estetrol component suitable for the dosage unit or the cosmetic or medical uses and methods of treatment described herein is estetrol, including hydrates thereof. Most preferably, said estetrol component is estetrol monohydrate.

The term “estetrol” as used herein refers to 1,3,5 (10)-estratrien-3,15alpha, 16alpha, 17beta-tetrol or 15alpha-hydroxyestriol as well as hydrates of estetrol, e.g. estetrol monohydrate. “Estetrol”, or short “E4” is an estrogen steroid produced by the foetal human liver (PubChem CID: 27125). Estetrol may be described as a 3-hydroxy steroid corresponding to 17beta-estradiol wherein the 15a and 16a positions are substituted for two additional hydroxy groups. It is known that estetrol is an estrogen receptor agonist (Coelingh Bennink et al., Estetrol review: profile and potential clinical applications, Climacteric, 2008). In instances wherein the estetrol component described herein indicates estetrol, said estetrol may be endogenous estetrol. Alternatively, the estetrol may be chemically synthesised, synthesised by the use of (mutant) recombinant enzymes, or synthesised by any combination thereof. Estetrol may alternatively be indicated in the art by its molecular formula: C₁₈H₂₄O₄, or by structural formula (I):

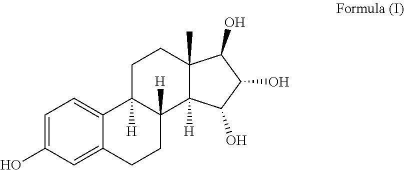

“(w/w)”, or alternatively indicated throughout the art by terms such as “weight by weight” or “weight for weight”, refers to the contribution of a particular molecule or substance within a composition or mixture as measured by its weight (i.e., mass).

In particular embodiments, the composition comprises of from about 0.01% to about 0.18% (w/w) of an estetrol component such as for example estetrol. In preferred embodiments, the composition comprises of from about 0.02% to about 0.16% (w/w) of an estetrol component such as for example estetrol, preferably of from about 0.03% to about 0.14% (w/w) of an estetrol component such as for example estetrol, more preferably of from about 0.04% to about 0.12% (w/w) of an estetrol component such as for example estetrol, yet more preferably of from about 0.05% to about 0.10% (w/w) of an estetrol component such as for example estetrol, even more preferably of from about 0.05% to about 0.08% (w/w) of an estetrol component such as for example estetrol.

In alternative particular embodiments, the composition comprises of about 0.18% (w/w) or less of an estetrol component such as for example estetrol. In preferred embodiments, the composition comprises of about 0.16% (w/w) or less of an estetrol component such as for example estetrol, preferably of about 0.14% (w/w) or less of an estetrol component such as for example estetrol, more preferably of about 0.12% (w/w) or less of an estetrol component such as for example estetrol, yet more preferably of about 0.10% (w/w) or less of an estetrol component such as for example estetrol, even more preferably of about 0.08% (w/w) or less of an estetrol component such as for example estetrol.

Optionally, the composition comprises of about 0.04% to 1% (w/w) of estetrol. In certain embodiments, the composition comprises of about 0.05% to 0.5% (w/w) of estetrol. In further embodiments, the composition comprises about 0.06% to 0.5% (w/w) of estetrol.

In preferred embodiments, the estetrol is present or used herein as a monohydrate. Hence, in particular embodiments the pharmaceutical composition comprises of from about 0.01% to about 0.18% (w/w) of estetrol monohydrate. In preferred embodiments, the composition comprises of from about 0.02% to about 0.16% (w/w) of estetrol monohydrate, preferably of from about 0.03% to about 0.14% (w/w) of estetrol monohydrate, more preferably of from about 0.04% to about 0.12% (w/w) of estetrol monohydrate, yet more preferably of from about 0.05% to about 0.10% (w/w) of estetrol monohydrate, even more preferably of from about 0.05% to about 0.08% (w/w) of estetrol monohydrate.

In alternative particular embodiments, the pharmaceutical composition comprises of about 0.18% (w/w) or less of estetrol monohydrate. In preferred embodiments, the composition comprises of about 0.16% (w/w) or less of estetrol monohydrate, preferably of about 0.14% (w/w) or less of estetrol monohydrate, more preferably of about 0.12% (w/w) or less of estetrol monohydrate, yet more preferably of about 0.10% (w/w) or less of estetrol monohydrate, even more preferably of about 0.08% (w/w) or less of estetrol monohydrate.

The above embodiments do not exclude the presence of non-estetrol estrogenic components in the pharmaceutical composition. Also envisaged are different estetrol components within a single composition. In such embodiments, the composition may by means of illustration and not limitation comprise both estetrol or, more particularly, estetrol monohydrate and an ester of estetrol.

With “topical application” application to a particular place on or in the body is meant. In particular, application to body surfaces such as the skin or mucous membranes is envisaged. When used topically, local effects are usually sought, particularly local effects on the skin and mucous membranes. While topical application also includes application to the vagina, topical application is preferably understood as application to the skin within the present context.

In particular embodiments, the pharmaceutical composition comprises a permeation enhancer that enables permeation through the stratum corneum. In the context of the present invention, the term “permeation enhancer”, interchangeably used with terms including but not limited to “permeability enhancer”. “permeability increaser”. “permeability inducer”, and even “skin enhancer”. It is appreciated by a skilled person that the term “permeation enhancer” upon use throughout the present disclosure indicates a permeation enhancer molecule as part of a solvent or solvent system. Different permeation enhancers have been described in detail in the art and include without limitation those listed in the CPE database (Vasyuchenko et al., Pharmaceutics. 2021). Exemplary and non-limiting permeation enhancers are described further herein.

The composition and different cellular architectural layers of the skin are known. A skilled person appreciates that the human skin can generally be considered to comprise three distinct layers: the epidermis, the dermis, and the hypodermis. The epidermis is the upper layer of the skin which comprises mainly of keratinocytes, i.e. epithelial cells that proliferate and differentiate to eventually generate the stratum corneum (outermost layer of dead skin cells). The epidermis forms a barrier to environmental pathogens such as bacteria, regulates the amount of water released from the body and plays a predominant role in wound healing. The second skin layer, i.e., the dermis (alternatively “corium” or “skin connective tissue”) is situated between the hypodermis and epidermis and mainly comprises (mesenchymal) fibroblasts. The dermis is tightly connected to the epidermis by means of a basement membrane, i.e., a sheet-like type of extracellular matrix. The dermis is also considerably thicker than the epidermis and the fibroblasts in the dermis produce the extracellular matrix (collagen, glycosaminoglycans including hyaluronic acid, elastic fibres . . . ). The main roles of the dermis are maintaining skin thickness and elasticity, maintaining skin hydration (through the water-holding capacity of glycosaminoglycans, including hyaluronic acid) and wound healing, i.e. by reforming and remodelling the damaged extracellular matrix. The deepest layer of the skin is commonly indicated as the “hypodermis (layer)”, interchangeably annotated in the art by terms such as “subcutaneous tissue” and “hypoderm”. “subcutis”, and “superfacial fascia”.

Therefore, a skilled person appreciates that “stratum corneum” to which reference is made herein refers to the outer layer of the epidermis that is composed of multiple layers of terminally differentiated keratinocytes made primarily of the proteinaceous material keratin. The composition, function, and particulars of the stratum corneum have been described in great detail in the art (e.g. in Matsui and Amagai. Int Immunol. 2015). It is generally considered that permeation of pharmaceutically active agents throughout the skin is rate limited by the stratum corneum. Thus, the permeation enhancers envisaged by the present invention aid in permeation of at least the estetrol component throughout the stratum corneum and/or wound eschar. Permeation is particularly enabled in burn wounds.

The term “Pharmaceutically active ingredient”, interchangeably used throughout the present disclosure with “pharmaceutically active agent” is to be interpreted according to the definition of the term by the World Health organisation: “a substance used in a finished pharmaceutical product (FPP), intended to display pharmacological activity or to otherwise have direct effect in the diagnosis, cure, mitigation, treatment or prevention of disease, or to have direct effect in restoring, correcting or modifying physiological functions in human beings”.

The compositions described herein are particularly suited for use in wound healing and may therefore be interchangeably be referred to as “pharmaceutical compositions” in any given instance. Preferably, the compositions described herein are used for topical wound healing. The expression “used for topical wound healing” as referred to herein relates to the treatment of one or more wounds of a subject, wherein the area of the wound can be defined by a certain surface area. Furthermore, the expression indicates the use of the compositions described herein for treatment of wounds of the skin of a subject. The term “topical wound healing” further indicates localized administration (i.e. application) of the composition described herein to the wound area. Topical administration also may involve the use of transdermal administration means such as but not limited to transdermal patches, as discussed further throughout the present disclosure.

The terms “wound area” and “wound size” as used herein refer to a physical measure of disruption of the normal continuity of structures. The wound area or wound size can e.g. be expressed in square cm. Depending on the wound size or area, the concentration of the composition to be applied on said wound may have to be adjusted in order not to exceed the maximally tolerated dose. The composition can in such a case be prepared specifically for said subject or one can have different grades or concentrates pre-prepared. For a larger wound it is therefore advisable to use the compositions according to the invention in lower concentrations, while application on smaller wounds allows higher concentrations to be used. If the results from the mouse studies are extrapolated to conditions in a human, it can be calculated that the 0.06% (w/w) concentration in mice corresponds to 0.1 mg of the estetrol component in a hydrogel to be applied to a human subject. Hence the topical application of an estetrol component of about 0.1 mg to a subject can be assumed to have no systemic effects, for example the thickening of the uterus in a female subject. The higher doses of 0.5% (w/w) in mice can be extrapolated to 0.9 mg or about 1 mg estetrol that can be topically administered with no significant risk of systemic effect, or at least with less systemic effect than the commercially available Estrogel®; comprising 0.06% estradiol and which is regulatory approved for topical application.

The terms “subject”. “individual” or “patient” can be used interchangeably herein, and typically and preferably denote humans, but may also encompass reference to non-human animals, preferably warm-blooded animals, even more preferably mammals, such as, e.g., non-human primates, rodents, canines, felines, equines, ovines, porcines, and the like. The term “non-human animals” includes all vertebrates, e.g., mammals, such as non-human primates. (particularly higher primates), sheep, dog, rodent (e.g. mouse or rat), guinea pig, goat, pig, cat, rabbits, cows, and non-mammals such as chickens, amphibians, reptiles etc. In certain embodiments, the subject is a non-human mammal. Preferred subjects are human subjects including all genders and all age categories thereof. Both adult subjects, new-born subjects, and foetuses are intended to be covered by the term “subject”. Thus, both adult and new-born subjects are intended to be covered. Examples of subjects include humans, dogs, cats, cows, goats, and mice. Preferred subjects in the context of the invention are defined further below.

In another yet related aspect, the invention concerns an estetrol component for use in wound healing, more particularly localized or topical wound healing. Hence, the present invention envisages the use of an effective amount of an estetrol component for use in wound healing by applying an estetrol component to the skin or wound area of the subject. The term “an effective amount” refers to an amount necessary to obtain a physiological effect. The physiological effect may be achieved by one dose or by repeated doses. In certain embodiments, the invention concerns an estetrol component in presence of a permeation enhancer enabling permeation through the stratum corneum for use in wound healing. In further embodiments, the invention concerns a pharmaceutical composition comprising an effective amount of an estetrol component and a permeation enhancer for use in wound healing.

Preferably, the estetrol component for use in wound healing is estetrol, most preferably estetrol monohydrate. In alternative embodiments, the invention concerns a pharmaceutical composition comprising an effective amount of an estetrol component in conjunction with a second composition comprising a permeation enhancer for use in wound healing. In such embodiments, the particular order of administration of the first and second composition to the wound area (i.e. wound site) is not particularly limiting. Hence, the compositions may be applied either sequentially or (near) simultaneously.

In certain embodiments, the pharmaceutical composition is for use in wound healing and comprises of from about 0.01% to about 5% (w/w), such as from about 0.08 to about 1.2% (w/w), from about 0.09% to about 1.1% (w/w), or from about 0.1% to about 1% (w/w) of an estetrol component (such as estetrol, preferably estetrol monohydrate) and a permeation enhancer, preferably of from about 0.02% to about 2.5% (w/w) of an estetrol component and a permeation enhancer, more preferably of from about 0.02% to about 2% (w/w) of an estetrol component and a permeation enhancer, more preferably of from about 0.03% to about 1.5% (w/w) of an estetrol component and a permeation enhancer, yet more preferably of from about 0.03% to about 1% (w/w) of an estetrol component and a permeation enhancer, yet more preferably of from about 0.03% to about 0.75% (w/w) of an estetrol component and a permeation enhancer, yet even more preferably of from about 0.04 to about 0.5% (w/w) of an estetrol component and a permeation enhancer, most preferably about 0.06% (w/w) of an estetrol component and a permeation enhancer.

In alternative embodiments, the pharmaceutical composition is for use in wound healing and comprises of from about 0.01% to about 5% or less (w/w), such as from about 0.08 to about 1.2% (w/w), from about 0.09% to about 1.1% (w/w), or from about 0.1% to about 1% (w/w) of an estetrol component (such as estetrol, preferably estetrol monohydrate) and a permeation enhancer, preferably about 2.5% (w/w) or less of an estetrol component and a permeation enhancer, more preferably about 2% (w/w) or less of an estetrol component and a permeation enhancer, more preferably about 1.5% (w/w) or less of an estetrol component and a permeation enhancer, yet more preferably about 1% (w/w) or less of an estetrol component and a permeation enhancer, vet more preferably 0.75% (w/w) or less of an estetrol component and a permeation enhancer, yet even more preferably about 0.5% (w/w) or less of an estetrol component and a permeation enhancer, most preferably about 0.1% (w/w) or less of an estetrol component and a permeation enhancer.

Preferably, the pharmaceutical composition referred to herein is a hydrogel, or is comprised in a hydrogel. Gels are semi-solid systems in which liquids are solidified by gel skeleton formers. In a hydrogel, the liquid that forms the gel is water or an aqueous solution. The term “hydrogel” as used herein refers to an aqueous solution of active ingredients that is mainly solidified with macromolecular hydrophilic substances to form a gel. Macromolecular hydrophilic substances and thus polymeric materials swell when they come into contact with water, which, depending on the concentration, results in solutions with pseudoplastic flow behavior or plastic structures that contain a necessary aqueous component. Thus, hydrophilic gels consist of water or aqueous solutions that are usually gelled with hydrophilic macromolar compounds. Gels built up with hydrophilic macromolar scaffolds are generally thixotropic. In contrast to creams, gels are referred to as true single-phase systems.

Hydrogels improve or restore the moisture balance to a wound bed by balancing hydration with absorption of excessive fluid. Hydrogels as envisaged herein may comprise any suitable polymer or combination of polymers, such as but not limited to hydrophilic polymers, acrylic acid, acrylamide, and 2-hydroxyethylmethacry late.

Hydrogels are particularly preferred forms of the compositions disclosed herein in view of their capacity to provide moisture balance to a wound bed by balancing hydration with absorption of excessive fluid. Examples of hydrogels include, but are not limited to, synthetic hydrogels, stimuli-sensitive hydrogels. (poly) peptide based hydrogels, hybrid hydrogels, and DNA based hydrogels. Production methods for each of these hydrogel categories have been described in the art and are therefore known to a skilled person.

By means of illustration and not limitation, examples of synthetic hydrogels include double network hydrogels. Examples also include nanocomposite hydrogels. Stimuli-sensitive hydrogels are characterised by their capacity to undergo changes in swelling that may be mediated by external stimuli (e.g., pH, temperature, ionic strength, solvent type, electric field, magnetic field, light, and chelating species). Examples of stimuli-sensitive hydrogels include without limitation hydrogels formed from block co-polypeptides recombinant segments of natural structural proteins such as elastin, silk, silk-like, and elastin-like peptide blocks, and recombinant triblock copolymers of one or more polypeptide sequences.

The term “hybrid hydrogels” is used herein to indicate hydrogels comprising components from at least two distinct classes of molecules, such as for example synthetic polymers and biological macromolecules, interconnected either covalently or non-covalently.

In the gel formulation as described herein, the presence of some excipients such as propylene glycol, glycerol. Transcutol®, benzyl alcohol, and PEG400) can increase the solubility of estetrol. The use of these excipients in the gel formulation allows estetrol to be soluble and completely available to permeate through the membrane or, similarly, through the stratum corneum and/or the wound eschar and/or into burn wounds. This contributes to an increase in the rate of diffusion across the membrane or, similarly, through the stratum corneum and/or the wound eschar and/or into burn wounds as predicted by Fick's law of diffusion, since a higher estetrol concentration gradient is produced by increasing estetrol solubility between the two sides of the membrane.

The cream-diffusion profile showed a slower release of estetrol in the same time when compared of gel formulations. In a cream formulation, the estetrol component is likely located inside the internal phase of the emulsion and needs to diffuse towards the external phase of the emulsion before being able to diffuse through the barrier membrane or, similarly, through the stratum corneum and/or the wound eschar and/or into burn wounds.

However, the high partition coefficient of the cream's base prevents estetrol from diffusing rapidly along its concentration gradient. The whole process is slower and the release rate of estetrol decreases dramatically.

It is understood that any reference to “composition” and “pharmaceutical composition” encompasses any hydrogels described herein, and vice versa.

Preferably, the hydrogel envisaged herein comprises a permeation enhancer that enables permeation through the stratum corneum. More preferably, the hydrogel referred to herein is a hydrogel comprising of from about 0.02% to about 10% (w/w) of an estetrol component such as estetrol, preferably estetrol monohydrate. Preferably, the hydrogel referred to herein is a hydrogel comprising of from about 0.02% to about 5% (w/w) of an estetrol component, more preferably, the hydrogel referred to herein is a hydrogel comprising of from about 0.02% to about 2.5% (w/w) of an estetrol component, more preferably the hydrogel comprises of from about 0.03% to about 0.75% (w/w) of an estetrol component, preferably of from about 0.04% to about 0.5% (w/w) of an estetrol component, more preferably of from about 0.05% to about 0.25% (w/w) of an estetrol component, such as from about 0.08 to about 1.2% (w/w), from about 0.09% to about 1.1% (w/w), or from about 0.1% to about 1% (w/w), most preferably the hydrogel comprises of from about 0.06% (w/w) of an estetrol component.

Alternatively, the hydrogel referred to herein is a hydrogel comprising about 10% (w/w) or less of an estetrol component. Preferably, the hydrogel referred to herein is a hydrogel comprising about 5% (w/w) or less of an estetrol component, more preferably, the hydrogel referred to herein is a hydrogel comprising about 2.5% (w/w) or less of an estetrol component, more preferably the hydrogel comprises about 0.75% (w/w) or less of an estetrol component, preferably about 0.5% (w/w) or less of an estetrol component, more preferably about 0.25% (w/w) or less of an estetrol component, more preferably about 0). 1% or less (w/w) of an estetrol component, most preferably about 0.08% or less (w/w) of an estetrol component.

In particular embodiments wherein the (pharmaceutical) composition is a hydrogel, said hydrogel is characterised by a favourable release profile, e.g. when compared to other formulations such as, but not limited to creams. In preferred embodiments, the hydrogel described herein is characterised by a mean cumulative amount of estetrol component released of at least about 2.5 μg/cm², at least about 5 μg/cm², at least about 7 μg/cm², at least about 10 μg/cm², ate least about 15 μg/cm², at least about 20 μg/cm², at least about 25 μg/cm², preferably at least about 50 μg/cm², more preferably at least 100 μg/cm², more preferably at least about 150 μg/cm², more preferably at least about 200 μg/cm² within 1 hour across an isopore membrane in a receptor solution of 40:30:30 v/v/v ethanol:PEG400:water. In alternative embodiments, the hydrogel described herein is characterised by a mean cumulative amount of estetrol component released of from about 25 μg/cm² to about 200 μg/cm² within 1 hour across an isopore membrane in a receptor solution of 40:30:30 v/v/v ethanol:PEG400:water. Depending on the surface of the wound, the concentration of the estetrol component may need to be reduced in order not to result in a cumulative administration of estetrol in too high amounts. This can be easily calculated by a physician or pharmacist. In preferred alternative embodiments, the hydrogel described herein is characterised by a mean cumulative amount of estetrol component released of from about 25 μg/cm² to about 100 μg/cm² within 1 hour across an isopore membrane in a receptor solution of 40:30:30 v/v/v ethanol:PEG400:water. In further preferred alternative embodiments, the hydrogel described herein is characterised by a mean cumulative amount of estetrol component released of from about 25 μg/cm² to about 50 μg/cm² within 1 hour across an isopore membrane in a receptor solution of 40:30:30 v/v/v ethanol:PEG400:water.

Preferably, the hydrogel described herein is characterised by a mean cumulative amount of estetrol component released of at least about 350 μg/cm², preferably at least about 400 μg/cm², more preferably at least about 450 μg/cm² within 8 hours across an isopore membrane in a receptor solution of 40:30:30 v/v/v ethanol:PEG400:water.

Preferably, the hydrogel described herein is characterised by a mean % applied dose of the estetrol component into a receptor solution of 40:30:30 v/v/v ethanol:PEG400:water of at least about 15%, preferably at least about 20% after 1 hour and/or a mean % applied dose of the estetrol component into a receptor solution of 40:30:30 v/v/v ethanol:PEG400:water of at least about 40%, preferably at least about 50%, more preferably at least 80% after 8 hours. In alternative embodiments, the hydrogel described herein is characterised by a mean % applied dose of the estetrol component into a receptor solution of 40:30:30 v/v/v ethanol:PEG400:water of from about 15% to about 30% after 1 hour and/or a mean % applied dose of the estetrol component into a receptor solution of 40): 30:30 v/v/v ethanol:PEG400:water of from about 45% to about 90% after 8 hours.

In highly preferred embodiments, the hydrogel described herein is characterised by an estetrol component release rate (i.e., slope) of from about 2.5 μg/cm²/√h, at least about 5 μg/cm²/√h, at least about 7 μg/cm²/√h, at least about 10 μg/cm²/h, ate least about 15 μg/cm²/√h, at least about 20 μg/cm²/√h, at least about 25 μg/cm²/√h to about 50 μg/cm²/√h and a mean cumulative amount of estetrol component released of from about 50 μg/cm² to about 100 μg/cm² after 8 hours, and optionally a percentage of estetrol amount released of from about 75% to about 95% across an isopore membrane in receptor solution 40:30:30 v/v/v ethanol:PEG400:water. Yet more preferably, the hydrogel described herein is characterised by an estetrol component release rate (i.e., slope) of from about 30 μg/cm²/√h to about 35 μg/cm²/√h and a mean cumulative amount of estetrol component released of from about 75 μg/cm² to about 90 μg/cm² after 8 hours, and optionally a percentage of estetrol amount released of from about 85% to about 90% across an isopore membrane in receptor solution 40:30:30 v/v/v ethanol:PEG400:water. Most preferably, the hydrogel described herein is characterised by an estetrol component release rate (i.e., slope) of from about 33 μg/cm²/√h to about 34 μg/cm²/√h and a mean cumulative amount of estetrol component released of from about 82 μg/cm² to about 83 μg/cm² after 8 hours, and optionally a percentage of estetrol amount released of from about 86% to about 88% across an isopore membrane in receptor solution 40:30:30 v/v/v ethanol:PEG400:water.

In embodiments wherein the composition is a cream, said cream may be characterised by an estetrol component release rate (i.e., slope) of from about 2.5 μg/cm²/√h, at least about 5 μg/cm²/√h, at least about 7 μg/cm²/√h, at least about 10 μg/cm²/h, ate least about 15 μg/cm²/√h, at least about 20 μg/cm²/h, or from 15 μg/cm²/√h to about 75 μg/cm²/√h across an isopore membrane in receptor solution 40:30:30 v/v/v ethanol:PEG400:water. Preferably, said cream may be characterised by an estetrol component release rate (i.e., slope) of from about 25 μg/cm²/√h to about 55 μg/cm²/√h across an isopore membrane in receptor solution 40:30:30 v/v/v ethanol:PEG400:water.

The cream may further be characterised by a mean cumulative amount of estetrol component released of from about 1 μg/cm² to about 100 μg/cm² after 1 hour across an isopore membrane in receptor solution 40:30:30 v/v/v ethanol:PEG 400:water. Preferably, the cream may be characterised by a mean cumulative amount of estetrol component released of from about 5 μg/cm² to about 80 μg/cm² after 1 hour across an isopore membrane in receptor solution 40:30:30 v/v/v ethanol:PEG400:water. The cream may further be characterised by a mean cumulative amount of estetrol component released of from about 25 μg/cm² to about 150 μg/cm² after 8 hours across an isopore membrane in receptor solution 40:30:30 v/v/v ethanol:PEG400:water. Preferably, the cream may be characterised by a mean cumulative amount of estetrol component released of from about 50 μg/cm² to about 120 μg/cm² after 8 hours across an isopore membrane in receptor solution 40:30:30 v/v/v ethanol:PEG 400:water.

As is evident for a skilled person, the present disclosure encompasses the use of each of the particular forms of the pharmaceutical composition disclosed herein for use in wound healing. Hence, the present invention is also directed to a hydrogel as disclosed herein for use in wound healing.

While a hydrogel is a preferred form of the pharmaceutical composition disclosed throughout the present disclosure, this does not exclude other topical formulations known in the art. Suitable formulations therefore include, but are not limited to emulsions, suspensions, ointments, pastes, lotions, gels (including hydrogels), foams, mousses, sprays, and creams. Each of these terms are intended to correspond to their generally accepted meaning. Similarly, these topical formulations can either be applied directly to the skin as such, or in combination with a dressing, patch, bandage, band aid, tampon, the inside of a plaster, or the like to prevent the formulation from being removed off the skin and in some embodiments to shield the wound from external influences such as dirt and microorganisms.

“Emulsion” broadly refers to any mixture of at least two liquids that are unmixable (i.e., immiscible, unblendable) and thus wherein a first liquid is distributed in small droplets (dispersed phase) throughout a second liquid (dispersion medium). Therefore, in certain embodiments the pharmaceutical composition described herein is an oil-in-water or water-in-oil emulsion. Emulsions are widely used in skin care formulations and can be classified as creams and lotions. Related hereto. “suspension” broadly refers to a heterogeneous mixture containing solids dispersed in a liquid phase that are not dissolved and have a size which is sufficiently large to allow for sedimentation.

“Cream” generally refers to a water-in-oil emulsion wherein an aqueous phase is dispersed in an oil phase, but may equally be an oil-in-water emulsion in which an oil is dispersed within an aqueous base. It is generally accepted that creams differ from emulsions in that emulsions are stable suspensions of small immiscible droplets of fluid immiscible with another fluid part of the emulsion, while a cream instead indicates a particular subset of emulsions that are more viscous and usually include more lipophilic and/or surfactant components.

A “lotion” is a low- to medium-viscosity liquid composition. Generally, lotions are less viscous than creams, but in some cases the viscosity of both can be similar. A lotion can contain finely powdered substances that are insolubilin the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

An “ointment” broadly generally refers to a more viscous oil-in-water cream, i.e., to a semi-solid substance containing an ointment base and optionally one or more pharmaceutically active ingredients (in the context of the present invention an estetrol component). Examples of suitable ointment bases include hydrocarbon bases, absorption bases, water-removable bases, and water-soluble bases. “Pastes” generally differ from ointments in that they contain a larger percentage of solids. Overall, pastes are more absorptive and less greasy when compared to ointments based on an identical set of ingredients/excipients.

“Foam” as used herein refers to a dispersion of gas particles in a liquid medium. Oil-in-water emulsions, water-in-oil emulsions, ethanol, water, solvents, liquid oil, propylene glycol, and glycerine can be listed as examples of liquid media in a foam. It is appreciated by a skilled person that foams may be generated by reducing the surface tension of the liquid mixing in (a) gaseous substance(s), causing bubble formulation. The acceptability of the foams is due to the fact that they are easy to apply on large areas of the skin, does not leave an oily or greasy film. and have rapid absorption into the skin. “Mousse” refers to a substance closely resembling a foam, but is commonly used to denote substances that are less aqueous. “Spray” means that the drug-containing solution is filled in a device suitable for spraying the drug-containing solution and released in a mist with the help of pressure.

As indicated above, any of the pharmaceutical compositions such as but not limited to the hydrogels disclosed herein may comprise a permeation enhancer. Preferably, the permeation enhancer comprises a molecule that enables permeation through the stratum corneum (i.e. a permeation enhancing molecule) and a solvent or solvent system. Optionally, the permeation enhancer is present in the composition such as but not limited to the hydrogels disclosed herein in an amount of from about 0.5% to about 60% (w/w), preferably in an amount of from about 1% to about 50% (w/w), more preferably in an amount of from about 2.5% to about 45% (w/w), more preferably in an amount of from about 5% to about 40% (w/w), more preferably in an amount of from 10% to about 30% (w/w) or alternatively in an amount of from about 0.1% to about 5% (w/w). Optionally, the permeation enhancer molecule is present in the composition such as but not limited to the hydrogels disclosed herein in an amount of from about 0.1% to about 25% (w/w), preferably in an amount of from about 0.5% to about 15% (w/w), more preferably in an amount of from about 1% to about 10% (w/w), more preferably in an amount of from about 2.5% to about 7.5% (w/w), more preferably in an amount of from 3.5% to about 5% (w/w). Optionally, the solvent (system) is present in the composition such as but not limited to the hydrogels disclosed herein in an amount of from about 1% to about 60% (w/w), preferably in an amount of from about 5% to about 50% (w/w), more preferably in an amount of from about 10% to about 40% (w/w), more preferably in an amount of from about 15% to about 30% (w/w), more preferably in an amount of from 18% to about 25% (w/w).

The permeation enhancer described herein is not particularly limiting for the invention and may therefore comprise or consist of a molecule selected from the group consisting of suberin, lignin, cutin, include dimethyl sulfoxide, ethanol, propylene glycol, glycerin, propylethylene glycols, urea, dimethyl acetamide, sodium lauryl sulfate, poloxamers, spans, tweens, lecithin, terpenes, and combinations thereof. Preferred permeation enhancing molecules in the context of the present invention include ethanol, ethers, benzyl alcohols, fatty acids and esters thereof, or any combination thereof. Particularly preferred permeation enhancer molecules in the context of the present invention include Transcutol®, benzyl alcohol, and any combination thereof. Benzyl alcohol (C₆H₅CH₂OH) may be interchangeably referred to in the art as “phenylmethanol”. “phenylcarbinol”, and “benzenemethanol”. Transcutol® is a commonly accepted trade name for 2-(2-ethoxyethoxy) ethanol, also annotated interchangeably in the art by diethylene glycol monoethyl ether (C₆H₁₄O₃). Optionally, the pharmaceutical composition described herein comprises as permeation enhancer of from about 1% to about 20% Transcutol® (w/w), preferably of from about 2% to about 10% Transcutol® (w/w), more preferably of from about 3% to about 8% Transcutol® (w/w), most preferably of from about 4% to about 6% Transcutol® (w/w).

The permeation enhancers of the present invention comprise a permeation enhancer molecule and a solvent or solvent system. Preferred solvents of the permeation enhancer include but are not limited to polyethylene glycols (PEG), propylene glycol (PG), and combinations thereof. “Polyethylene glycol” may be interchangeably indicated by terms such as but not limited to polyethylene oxide or poly(oxyethylene), poly(ethylene oxide), and polyoxyethylene have been described in detail in the art and are therefore known to a skilled person, who appreciates the polyethylene glycol is characterised by the chemical formula H—(O—CH₂—CH₂)ₙ—OH wherein n is an integer. Polyethylene glycols are polyether compounds derived from petroleum. Preferred PEGs are PEGs characterised by a molecular weight of from between about 150 g/mol to about 5000 g/mol, more preferably of from between about 200 g/mol to about 2500 g/mol, vet more preferably between about 250 g/mol to about 1000 g/mol, most preferably between about 300 g/mol to about 600 g/mol. Therefore, the PEG referred to herein may be selected from the group consisting of: PEG200, PEG300, PEG400, PEG500. PEG600, and any combination thereof. Most preferably, the permeation enhancer comprises as solvent or part of the solvent a PEG having a molecular weight of about 400 g/mol such as but not limited to PEG400. Propylene glycol, commonly annotated in the art as propane-1,2-diol, α-propylene glycol, 1,2-propanediol, 1,2-dihydroxypropane. Propylene glycol is characterised by the chemical formula CH₃CH(OH)CH₂OH.

Preferably, the permeation enhancer comprises of from about 5% to about 50% (w/w), preferably from about 5% to about 35% (w/w) or from about 10% to about 45% (w/w) PEG, preferably PEG400), and/or of from about 10% to about 35% (w/w) PG. More preferably, the permeation enhancer comprises of from about 10% to about 30% (w/w) PEG, preferably PEG400), and/or of from about 12% to about 30% (w/w) PG. More preferably, the permeation enhancer comprises of from about 12% to about 25% (w/w) PEG, preferably PEG400, and/or of from about 15% to about 25% (w/w) PG. More preferably, the permeation enhancer comprises of from about 14% to about 23% (w/w) PEG, preferably PEG400), and/or of from about 16% to about 23% (w/w) PG. More preferably, the permeation enhancer comprises of from about 16% to about 22% (w/w) PEG, preferably PEG400, such as from about 18% to about 22% (w/w) PEG or from about 16% to about 20% (w/w) PEG, and/or from about 18% to about 22% (w/w) PG.

Optionally, the pharmaceutical composition (which optionally is a hydrogel) comprises benzyl alcohol. The term “benzyl alcohol” is to be interpreted according to its common interpretation in the art and therefore indicates an aromatic alcohol characterised by the chemical formula C₆H₅CH₂OH. If present, the amount of benzyl alcohol in the composition is not particularly limiting. However, preferably the amount of benzyl alcohol is of from about 0.1% to about 10% (w/w), more preferably of from about 0.5% to about 5% (w/w), more preferably of from about 1% to about 3% (w/w), vet more preferably of from about 1.5% to about 2.5% (w/w).

Optionally, the pharmaceutical composition (which may be a hydrogel) comprises a thickener. A “thickener” may be alternatively indicated by terms such as but not limited to “thickening agent” and “viscosity agent”. “thickener” as used in the context of the present invention indicates any substance or molecule that upon addition to a liquid or semi-solid composition increases the viscosity and/or texture of said composition. Hence, if present, the thickener may be selected from the group consisting of: carboxylic acid polymers, crosslinked polyacrylate polymers, polyacrylamide polymers, polysaccharides, diblock polymers, triblock polymers, gums, and any combination thereof. In embodiments wherein the thickener is or comprises a polysaccharide, said polysaccharide may be selected from the group consisting of: cellulose, cellulose derivatives, carboxymethyl cellulose, cellulose acetate propionate carboxylate, hydroxyethyl cellulose, hydroxyethyl ethylcellulose, hydroxypropyl cellulose, hydroxypropyl methylcellulose, methyl hydroxyethyl cellulose, hydroxyalkylated cellulose, lignin, cutin, suberin, microcrystalline cellulose, sodium cellulose sulfate, scleroglucans, and any combination thereof. Suitable gums that may act as thickener include acacia, agar, algin, alginic acid, cetyl alcohol, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and any combinations thereof.

In certain embodiments, the pharmaceutical composition described herein (which optionally is a hydrogel) comprises a thickener as described herein in an amount of from about 0.1% to about 25% (w/w), preferably in an amount of from about 0.3% to about 20% (w/w), more preferably in an amount of from about 0.4% to about 15%, more preferably in an amount of from about 0.5 to about 10% (w/w), more preferably in an amount of from about 0.75% to about 5% (w/w), or in an amount of from about 0.3% to about 3% (w/w). Preferred thickeners in the context of the present invention include but are not limited to thickeners selected from the group consisting of: hydroxyethyl cellulose (HEC), carboxymethyl cellulose (CMC), high molecular weight cross-linked acrylic based polymer, non-ionic triblock copolymer, or any combination thereof. More preferred thickeners in the context of the present invention include but are not limited to hydroxyethyl celluloses, high molecular weight cross-linked acrylic based polymers, non-ionic triblock copolymer, and any combination thereof. A preferred high molecular weight cross-linked acrylic based polymer is Carbomer, interchangeably indicated throughout the art by the trade name Carbopol®. Both Carbopol® homopolymers (i.e. acrylic acid crosslinked with allyl sucrose or allyl pentacrythritol). Carbopol® copolymers (acrylic acid and C10-C30 alkyl acrylate crosslinked with allyl pentaerythritol), Carbopol® interpolymers (carbomer homopolymer or copolymer that contains a block copolymer of polyethylene glycol and a long chain alkyl acid ester) are envisaged. A particularly preferred Carbopol® in the context of the present invention is Carbopol® 980, alternatively indicated throughout the art as “Carbomer Homopolymer Type C USP NF”, which is an homopolymer of acrylic acid crosslinked with allyl sucrose or allyl pentaerythritol in cosolvent of cyclohexane and ethyl acetate having a viscosity of 40000 to 60000 cP. A preferred hydroxyethyl cellulose is HEC250 HHX. A preferred non-ionic triblock copolymer is a non-ionic triblock copolymer having a molecular mass of from about 1800 g/mol and about 4000 g/mol and a polyoxyethylene content of from about 70% to about 80%. Highly preferred non-ionic triblock copolymers include poloxamer 188, poloxamer 407, or combinations thereof.

In certain embodiments, the thickener is or comprises HEC and is present in the pharmaceutical composition in an amount of from about 0.1% to about 10% (w/w), preferably in an amount of from 0.2% to about 7.5% (w/w), more preferably in an amount of from about 0.5% to about 5% (w/w), more preferably in an amount of from about 0.75% to about 2.5% (w/w), most preferably in an amount of from about 1% to about 2% (w/w). In certain embodiments, the thickener is or comprises Carbopol® and is present in the composition in an amount of from about 0.1% to about 10% (w/w), preferably in an amount of from 0.2% to about 7.5% (w/w), more preferably in an amount of from about 0.5% to about 5% (w/w), more preferably in an amount of from about 0.75% to about 2.5% (w/w), most preferably in an amount of from about 0.75% to about 1.5% (w/w). In alternative embodiments. Carbopol® is present in the composition in an amount of from about 0.1% to about 1% (w/w). In certain embodiments, the thickener is or comprises poloxamer 188 and is present in the composition in an amount of from about 0.1% to about 30% (w/w), preferably in an amount of from about 1% to about 15% (w/w), more preferably in an amount of from about 2.5% to about 10% (w/w). In certain embodiments, the thickener is or comprises poloxamer 407 and is present in the composition in an amount of from about 0.1% to about 30% (w/w), preferably in an amount of from about 5% to about 25% (w/w), more preferably in an amount of from about 10% to about 20% (w/w).

In certain embodiments, the thickener is or comprises CMC and is present in the pharmaceutical composition in an amount of from about 0.1% to about 10% (w/w), preferably in an amount of from 0.2% to about 7.5% (w/w), more preferably in an amount of from about 0.5% to about 5% (w/w), more preferably in an amount of from about 0.75% to about 2.5% (w/w), most preferably in an amount of from about 1% to about 2% (w/w) or about 1.5% (w/w).

Optionally, the pharmaceutical composition (which is optionally a hydrogel) comprises a preservative. In particular embodiments, the composition comprises a preservative in an amount of from 0.5% to 20% (w/w), preferably in an amount of from 1% to about 10% (w/w), more preferably in an amount of from about 1% to about 3% (w/w). The exact preservative is not particularly limiting for the invention and may therefore be selected from the group consisting of: lysozyme, nisin, quaternary ammonium preservatives, parabens, phenoxyethanol, benzyl alcohol, chlorobutanol, phenol, sorbic acid, thimerosal, natural preservatives, and any combination thereof. A preferred preservative in the context of the invention is benzyl alcohol.

Optionally, the pharmaceutical composition (which may be a hydrogel) comprises an emollient. “Emollient” as used throughout the present disclosure refers to a material useful for preventing and/or treating a dryness of the skin, as well as providing an extra protection of the skin. The particular emollient is not particularly limiting for the invention and may therefore be selected from the group consisting of: glycerol, acetyl alcohols, stearyl alcohol, stearic acid, isopropyl palmitate, squalene, lanolin, glycerin, petrolatum, petroleum, and any combination thereof. A particularly preferred emollient in the context of the present invention is glycerol. The emollient may be present in the composition in an amount of from about 2.5% to about 30% (w/w), preferably in an amount of from about 5% to about 25%, more preferably in an amount of from 7.5% to about 20% (w/w), more preferably in an amount of from about 8% to about 12% (w/w). In further embodiments, the emollient may be glycerol that is present in the composition in an amount of from about 2.5% to about 30% (w/w), preferably in an amount of from about 5% to about 25% (w/w), more preferably in an amount of from 7.5% to about 20% (w/w), more preferably in an amount of from about 8% to about 12% (w/w), most preferably in an amount of about 10% (w/w).

As described in detail throughout the present disclosure, any of the pharmaceutical compositions described herein may comprise a certain amount of components (i.e. pharmaceutically active agents and/or excipients) in addition to the estetrol component. In any of the embodiments of the invention, a solvent can be added to arrive at certain concentrations of said components. In further embodiments, an aqueous solution is used to complement the composition. The term “aqueous solution” refers to any solution comprising water or in which the solvent is water.

Additionally. “aqueous solution” is used to describe solutions displaying commonalities to water or watery solutions, not limited to characteristics such as appearance, smell, colour, taste, viscosity, pH, absorbance, or physical state under particular temperatures. In such embodiments, the aqueous solution may be water. In alternative further embodiments, a non-aqueous solution is used to complement the composition. In yet alternative embodiments, a mixture of a non-aqueous solution and an aqueous solution is used to complement the composition.

As defined herein, the pH of a composition, solution, or formulation may be measured using various methods as known to a person skilled in the art, pH indicators may be used that discolour by uptake or release of H+-ions, wherein their resulting colour is indicative for a certain pH value. Alternatively, pH meters may be used that measure the difference in electrical potential between a pH electrode and a reference electrode. The difference in electrical potential relates to the acidity or pH of the solution.

Considering the above, an exemplary pharmaceutical composition according to the invention comprises in addition to the estetrol component in any one of the concentrations listed herein:
from about 0.1% to about 60% (w/w) of a permeation enhancer.
from about 0.3% to about 20% (w/w) of a thickener:
optionally a preservative and/or an emollient:
water up to 100% (w/w).

In certain embodiments, the pharmaceutical composition consists essentially of, or consists of a permeation enhancer, a thickener, a preservative, and an emollient (in addition to the estetrol component). In preferred embodiments, the composition according to the invention comprises in addition to the estetrol component:
from about 0.1% to about 10% (w/w) of a permeation enhancer molecule, preferably from about 0.25% to about 8% (w/w) of a permeation enhancer molecule:
from about 10% to about 60% (w/w) of a permeation enhancer solvent:
from about 0.3% to about 20% (w/w) of a thickener, preferably from about 0.3% to about 10% (w/w) of a thickener, more preferably from about 0.3% to about 5% (w/w) of a thickener, most preferably from about 0.3% to about 3% (w/w) of a thickener.
optionally a preservative and/or an emollient:
water up to 100% (w/w).

In yet further preferred embodiments, the pharmaceutical composition according to the invention comprises in addition to the estetrol component in any one of the concentrations listed herein:
from about 1% to about 7.5% (w/w) of a permeation enhancer molecule, preferably from about 0.5% to about 5% (w/w) of a permeation enhancer molecule;
from about 15% to about 45% (w/w) of a permeation enhancer solvent:
from about 0.3% to about 20% (w/w) of a thickener, preferably from about 0.3% to about 10% (w/w) of a thickener, more preferably from about 0.3% to about 5% (w/w) of a thickener, most preferably from about 0.3% to about 3% (w/w) of a thickener.
optionally a preservative and/or an emollient:
water up to 100% (w/w).

In a particular embodiment, the pharmaceutical composition (optionally a hydrogel) comprises, consists essentially of, or consists of, in addition to the estetrol component in any one of the concentrations listed herein:
from about 8% to about 40%, preferably from about 16% to about 20%, most preferably about 18% (w/w) PEG400;
from about 9% to about 44%, preferably from about 18% to about 22%, most preferably about 20% (w/w) PG;
from about 4% to about 24%, preferably from about 8% to about 12%, most preferably about 10% (w/w) glycerol:
from about 0.5% to about 4%, preferably from about 1% to about 2%, most preferably about 1.5% (w/w) HEC; and
from about 0.75% to about 1.25%, preferably from about 1.5% to about 2.5%, most preferably about 2% (w/w) benzyl alcohol.

In a particular embodiment, the pharmaceutical composition (optionally a hydrogel) comprises, consists essentially of, or consists of, in addition to the estetrol component in any one of the concentrations listed herein:
from about 9% to about 44%, preferably from about 18 to about 22%, most preferably about 20% (w/w) PEG400;
from about 0.01% to about 2%, preferably from about 0.1% to about 1%, most preferably about 0.5% (w/w) Carbopol®; and
from about 0.1% to about 15%, 2% to about 12%, preferably from about 4% to about 6%, most preferably about 5% (w/w) Transcutol®.

In a particular embodiment, the pharmaceutical composition (optionally a hydrogel) comprises, consists essentially of, or consists of, in addition to the estetrol component in any one of the concentrations listed herein:
from about 10% to about 55%, preferably from about 30% to about 50%, most preferably from about 40% to 45% (w/w) PEG400;
from about 0.01% to about 2%, preferably from about 0.1% to about 1%, most preferably about 0.5% (w/w) Carbopol®; and
from about 0.1% to about 15%. 0.2% to about 10%, preferably from about 0.5% to about 5%, most preferably from about 1% to about 2.5% (w/w) Transcutol®.

The pharmaceutical composition described herein such as but not limited to the hydrogel described herein are envisaged for use as a medicament, both in a therapeutic and prophylactic context. More particularly, the composition described herein such as but not limited to the hydrogel described herein are envisaged for the medical use of wound healing. Alternatively worded, the invention relates to the use of a composition such as a hydrogel as described herein for the manufacture of a medicament for wound healing, preferably topical wound healing. Yet alternatively worded, the present invention relates to methods of treating wounds, preferably to methods of topical wound treatment, comprising administration of any one of the compositions or hydrogels described herein to a wound (site) of a subject. In the context of the present invention the composition such as the hydrogel described herein is used as a topical formulation, i.e. a local treatment means for wounds that is applied to the wound site.

As used throughout the present disclosure, the terms “therapy” or “treatment” refer to the alleviation or measurable lessening of one or more symptoms or measurable markers of a pathological condition, in the context of the present invention one or more wounds. The terms encompass both the therapeutic treatment of a wound that has already developed (i.e. an established wound), as well as prophylactic or preventive measures, wherein the goal of the treatment is to prevent occurrence and/or re-occurrence, development and progression of wounds in a subject, i.e., on the skin of a subject. By means of illustration and limitation, a preventive use of the pharmaceutical composition described herein may be administration to a skin site that appears fragile in an elderly subject to prevent bedsores. An alternative example may be application to a skin site of a subject that will form a surgical insertion site in a foreseeable future. Measurable lessening includes any statistically significant decline in a measurable inflammation marker, wound area, and/or wound depth and/or wound width. Statistically significant as used herein refers to p values below 0.05, which is a commonly accepted cut-off score in statistical analysis as a skilled person appreciates. More particular indications of a healing wound site are described in detail further below. Beneficial or desired clinical results of the medical use (i.e., treatment) may include, without limitation, alleviation of pain and/or discomfort, improvement of one or more biological markers, diminishment of extent of the wound, stabilized (i.e. not worsening) state of the wound, acceleration of wound healing progression, the improvement of the quality of life of the patient and the like.

A skilled person is aware that in order to achieve an effective therapeutic treatment, a therapeutically effective dose needs to be administered to said subject. Therefore, in the context of the present disclosure “an effective amount” refers to an amount necessary to obtain a physiological effect. The physiological effect may be achieved by a single dose or by multiple doses. A “therapeutically effective amount” or “therapeutically effective dose” indicates an amount of estetrol component that when administered brings about a clinical positive response with respect to treatment of a subject afflicted by one or more wounds. Similarly, a “prophylactically effective amount” or “prophylactically effective dose” refers to an amount of estetrol component that inhibits or delays wound onset or wound progression. A skilled person is aware that terms such as “quantity”. “amount” and “level” are synonyms and have a well-defined meaning in the art and appreciates that these in the context of the present application refer to a relative quantification of an estetrol component part of a pharmaceutical composition such as but not limited to a hydrogel, or when indicated refer to an absolute quantification of an estetrol component which is considered an effective amount for the applications described herein upon application to the skin of a subject.

Optionally, the pharmaceutical compositions and hydrogels described herein are for use in treatment of acute wounds. The cause of the acute wound is not particularly limiting for the invention, and therefore includes both wounds caused by injuries and surgically-induced wounds. The cause of the injury is not limiting for the invention and therefore encompasses both accidental injuries and injuries caused by malintent (i.e., combat wounds). Non-limiting examples of acute wounds include abrasions (i.e., scraped or rubbed away skin), incisions (i.e., clean cut wounds), lacerations (i.e., torn and/or ragged wounds), punctures (i.e., wounds having a relatively small wound opening produced by a relatively narrow-pointed object), and avulsions (pulled or teared skin wound).

Optionally, the pharmaceutical compositions and hydrogels described herein are for use in treatment of burn wounds. A skilled person appreciates that a “burn wound” refers to a particular kind of tissue injury caused by contact with heat, flame, chemicals, electricity, or radiation. First degree burns are mainly characterised by redness: second degree burns are characterised by the presence of one or more blistered spots (i.e., vesication): third degree burns are characterised by the presence of necrosis. Burns of the first and second degree are commonly referred to in the art as partial-thickness burns (i.e. destruction of tissue through the epidermis extending to but not through the dermis), while burns of the third degree are commonly referred to as full-thickness burns (i.e. destruction characterised by full extension through the dermis).

Optionally, the pharmaceutical compositions and hydrogels described herein are for use in treatment of chronic wounds. “chronic wounds” as referred to herein refer to any wound that is not succeeding, or has not succeeded to proceed through a standard wound healing process. Thus, wounds can be clinically categorized as acute or chronic based on their time frame of healing. Notably, surgical wounds can become chronic wounds and are referred to as surgical wounds that fail to heal by secondary intention. The term “chronic wound” may interchangeably be used with synonymous terms such as but not limited to “hard-to-heal wound”. “difficult-to-heal wound”. “non-healing wound”, and “complex wound”. Chronic wounds have been characterised in detail in the art (e.g. in Vanwijck. Bull Mem Acad R Med Belg. 2001). The healing process in chronic wounds may be dysregulated by a plethora of factors that prolong one or more stages wound healing phases. Non-limiting examples include without limitation infection, tissue hypoxia, necrosis, exudate, and excess levels of inflammatory cytokines. Commonly observed features of chronic wounds include a prolonged or uncontrolled inflammatory phase, persistent infections, formation of drug-resistant microbial biofilms, and the inability of dermal and/or epidermal cells to respond to reparative stimuli.

“Inflammation” as used herein refers broadly to the physiologic process wherein vascularized tissues respond to injury. Similarly, the term “inflammatory processes” refers to a process wherein soluble inflammatory mediators cooperate with cellular components in order to confine and remove any agents causing distress. The term “inflammatory mediators” refers broadly to any molecular mediator of the inflammatory process. Inflammatory mediators are capable of acting both locally at the site of tissue damage and/or infection, and at more distant sites. Certain inflammatory mediators are activated by the inflammatory process, while other inflammatory mediators are produced and/or released from cellular sources upon response to inflammation or upon activation by other inflammatory mediators. Examples of inflammatory mediators of the inflammatory response include, but are not limited to, plasma proteases, complement, kinins, clotting proteins, fibrinolytic proteins, lipid mediators, prostaglandins, leukotrienes, platelet-activating factor, peptides, amines, and proinflammatory cytokines.

Terms such as “inflammation of the skin” or “skin inflammation” used herein are to be interpreted according to the commonly accepted meaning in the state of the art and thus indicate any local immune response of the skin. The cause of the skin inflammation is generally occurrence of an injury such as a wound. Skin inflammation can therefore be considered the result of cellular interactions in the skin of a subject, with immune cells remaining the most important cell type. Skin inflammation referenced herein both indicates the “standard” inflammation observed in a wound, but equally indicates excessive inflammation that exceeds the normal boundaries of inflammation, and may be the consequence of a bacterial or fungal infection in the wound site, or a defective host response (eg. as in diabetes). Non-limiting examples of bacteria that may be involved in the infection of wounds include without limitation Staphylococcus aureus. Coagulase-negative staphylococci. Corynebacteria. Pseudomonas acruginosa, Proteus mirabilis, Escherichia coli, Acinetobacter baumanii, Serratia marcescens, Stenotrophonas maltophilia, Streptococcus agalactiae, Enterobacter cloacae, Enterococci, Klebsiella pneumoniae, Morganella morganii, Providencia stuarii, Alcaligenes faecalis, Citrobacter amalonaticus, Citrobacter koseri, Klebsiella oxytoca, Kocuria kristinae and Pseudomonas stutzeri. Non-limiting examples of fungi that may be involved in the infection of wounds include without limitation Candida albicans. Candida parapsilosis and Aspergillus niger.

A bacterium of particular interest in the context of the present invention is Klebsiella pneumoniae, which is known to act as a wound pathogen in infected wound sites such as but not limited to acute wounds (including surgical, injury, and combat wounds), burns, and chronic leg ulcers (Crompton et al., Lab Invest. 2016). Klebsiella pneumoniae has been associated in the art with reduced re-epithelialisation, increased proliferation, a heightened inflammatory response and perturbed wound matrix deposition. The inventors have found that the topical composition described herein is particularly suited for use in reducing inflammation in a Klebsiella pneumoniae infected wound, and wounds considered at risk to develop infection by Klebsiella pneumoniae.

Non-limiting examples of chronic wounds include vascular ulcers, pressure ulcers, and diabetic ulcers. Vascular ulcers encompass arterial ulcers and venous ulcers. Therefore, in certain embodiments the chronic wound is a wound selected from the group consisting of: arterial ulcers, venous ulcers, pressure ulcers, diabetic ulcers, and combinations thereof. “Venous ulcers” as used herein are caused by an increased venous pressure caused by venous valvular deficiencies. Pressure-induced changes in blood vessel wall permeability results in the leakage of fibrin and other plasma components into the perivascular location, with said accumulation of fibrin having a negative effect on wound healing. Collagen synthesis is downregulated by fibrin, resulting in pericapillary fibrin cuff formation which creates a barrier for normal vessel function, and confines blood-derived growth factors. The term “arterial ulcers” indicate chronic wounds that are the result of arterial insufficiency which may be caused by atherosclerosis or embolism, leading to a narrowing of the arterial lumen and ischemia, preventing timely healing of minor injuries. “Pressure ulcers” develop as a result of prolonged unrelieved pressure and shearing force applied to skin and the underlying muscle tissue, leading to a decrease in oxygen tension, ischemia reperfusion injury, and tissue necrosis. Finally. “diabetic ulcers” arise as a consequence of aging and diabetes. Diabetes may additionally worsen vascular pathologies which may in turn worsen arterial insufficiencies, venous insufficiencies, and/or pressure ulcers. Further aberrations leading to the development of diabetic ulcers in diabetic patients include neuropathy (often linked to vascular impairment), muscle metabolism deficiencies, and certain microvascular pathologies caused by hyperglycemia. Macroscopic pathologies seen in chronic, particularly diabetic, wounds generally include cellular phenotypic abnormalities, such as but not limited to low mitogenic, low motogenic potential, and an inability to respond to environmental factors.

In particular embodiments, the pharmaceutical composition (which may be a hydrogel) described herein is for use in wound healing of subjects that are characterised by an impaired wound healing. The impaired wound healing may arise due to an underlying pathology as described throughout the present disclosure and/or an infection of the wound. Infection is a common cause of delayed wound healing. Live bacteria (and subsequently produced bacterial toxins) induce excessive inflammatory responses and tissue damage. Potential consequences of bacterial infections of wounds include the occurrence of abscesses, cellulites, osteomyelitis, or limb loss (e.g., in diabetic patients). Moreover, inflammatory cells recruited to the wound site upon infection thereof produce proteases capable of degrading the extracellular matrix and growth factors present at the wound site. A considerable portion of wound-colonizing bacteria form are able to form biofilms, leading in turn to increased bacterial survival and increase production of virulence factors. Hence, in certain embodiments the subject is a subject characterised by a wound comprising a biofilm.

In certain embodiments, the pharmaceutical composition is used to treat wounds of subjects that are considered at risk of developing chronic wounds, or have developed at least one chronic wound in an earlier point in time.

In embodiments wherein the subject is a subject characterised by impaired wound healing, said impaired wound healing occurs at least about 10% slower, preferably at least about 20% slower, preferably at least about 30% slower, preferably at least about 40% slower, preferably at least 50% slower, preferably at least 60% slower, preferably at least 70% slower, preferably at least 80% slower, preferably at least 90% slower, when compared to wound healing in a subject that is not considered or suspected to have impaired wound healing (i.e. a subject which is considered healthy or generally healthy).

The particular manifestation of impaired wound healing and the cause thereof is not particularly limiting for the invention. Hence, in certain embodiments a phase of wound healing may be impaired in the subject selected from the group of wound repair phases consisting of: haemostasis (blood clotting), inflammation, proliferation (growth of new tissue), and maturation (tissue remodelling), or any combination thereof. The impaired wound healing process may be additionally characterised by the occurrence of infections, hypoxia, necrotic tissue, exudate (seeping out of cells and fluid from a wound), excessive levels of inflammatory cytokines, and any combinations thereof.

Optionally, the subject is characterised by an impaired wound healing due to deficient or absent blood clotting. Immediately after injury in healthy subjects, platelets attach to damaged blood vessels, initiate a release reaction, and initiate a haemostatic reaction. This results in a blood-clotting cascade that prevents excessive bleeding and provides provisional protection for the wounded area. Blood platelets have been described to release numerous growth factors, cytokines, and other survival or apoptosis-inducing agents. Crucial components of the platelet release reaction include platelet-derived growth factor (PDGF) and transforming growth factors A1 and 2 (TGF-A1 and TGF-2), which will attract inflammatory cells (e.g. leukocytes, neutrophils, and macrophages).

Optionally, the subject is characterised by an impaired wound healing due to a deficient inflammatory wound healing phase. In healthy subjects, the inflammatory phase is initiated in response to capillary damage, which leads to formation of a provisional blood clot matrix comprising, among other components, fibrin and fibronectin. Said provisional matrix fills wound area and triggers influx of effector cells. Platelets present in the clot release multiple cytokines that recruit inflammatory cells (e.g. neutrophils, monocytes, and macrophages, amongst others), fibroblasts, and endothelial cells.

Optionally, the subject is characterised by an impaired wound healing due to a defective proliferation phase. In healthy subjects, the proliferative phase is characterised by active angiogenesis thereby creating new capillaries that enable nutrient delivery to the wound site and support fibroblast proliferation. These fibroblasts synthesize and deposit extracellular matrix (ECM) components that replace the provisional matrix. Said fibroblasts are further characterised by contractile properties mediated by smooth muscle actin organized in microfilament bundles or stress fibres.

Optionally, the subject is characterised by an impaired wound healing due to a deficient remodelling phase. In healthy subjects, the final healing phase involves gradual remodelling of the granulation tissue and reepithelialisation. Proteolytic enzymes, such as matrix metalloproteinases (MMPs) and their inhibitors (TIMPs, tissue inhibitors of metalloproteinases) play a key role in the remodelling phase. During re-epithelialization, fibronectin and Type III collagen, the main components of the granulation tissue (i.e. the new stroma tissue), is substituted gradually by type I collagen and supplemented with elastin. Elastin contributes to skin elasticity and is initially absent from the granulation tissue. Finally, the cell density in the wound normalizes through apoptosis of vascular cells and fibroblasts.

Wound edge proliferation is a part of the wound re-epithelialisation process, the latter indicating the process of covering (i.e. resurfacing, providing) with new epithelium. In wounds of the skin, re-epithelialization has been documented to progress from the surrounding wound margins (i.e. wound edges) toward the centre of the wound. Re-epithelialization is part of the proliferation phase and generally initiates about 16 to about 24 hours after injury by activation of keratinocytes due to recruitment of neutrophils, monocytes, and macrophages to the wound site as detailed above. Activated keratinocytes are characterised by changes in the cytoskeleton and cell surface receptors of the cells. Moreover, activated keratinocytes are hyperproliferative and produce components of the dermal-epidermal junction. For example, activated keratinocytes produce matrix metalloprotease 9 (MMP-9), which causes degradation of the dermal-epidermal junction and allows said keratinocytes to migrate over the wound. Keratinocyte migration is an early event in wound re-epithelialization. Further study of activated keratinocytes in wounds led to the observation of a moving cohesive epithelial sheet at the edge of the wound which migrates towards the centre of the wound. Different mechanisms have been proposed for the process of keratinocyte migration over the wound bed, each envisaged and appreciated in the context of the present invention.

Optionally, the impaired wound healing can be observed and/or be apparent through reduced wound edge migration of the wound. “wound edge” as used herein refers to the outer circumference of a wound area, i.e. the portion of the wound area that is adjacent to non-injured tissue area of the skin of a subject. A person skilled in the art is capable of observing and/or measuring the wound area, and consequently the wound edge. Such observations and/or measurements may be conducted at multiple points in time to determine whether the wound of the subject is healing at a “normal” rate (i.e., a rate within the boundaries of what is considered common in healthy subjects) or whether any delayed onset or reduced rate of wound healing is occurring. Optionally, in absence of any treatment, the impaired wound healing in a wound healing impaired subject is manifested by a reduced wound edge migration of at least 25%, preferably at least 50%, more preferably at least 75%, or most preferably at least 100% when compared to a healthy subject.

In particular embodiments, the compositions and hydrogels of the above aspects are for use in improving re-epithelialization of a wound site. In the present context, the improved re-epithelialization may refer to the overall process of re-epithelialization, but equally to a certain improvement in a specific aspect thereof. Therefore in certain embodiments the improved wound healing upon treatment with the composition described herein may improve a process selected from the group consisting of: improved re-epithelialization of a wound site, increased proliferation of cells at a wound site, reduction of an inflammatory response at or in a wound site, improving matrix deposition at wound sites, hair follicle mediated re-epithelialization, re-epithelialization in partial thickness wounds from the bottom up, and any combinations thereof.

The improved re-epithelialization may be characterized by an increased and/or fastened production of provisional matrix. In such embodiments, the amount of provisional matrix that is produced may be increased by at least about 10%, preferably at least about 25%, preferably at least about 50%, preferably at least about 75%, more preferably at least about 100%, and/or the production of said provisional matrix may be fastened by at least about 10%, preferably at least about 25%, preferably at least about 50%, preferably at least about 75%, more preferably at least about 100% when compared to a subject that is not treated with the composition (optionally hydrogel) described herein.

Alternatively or in addition to the increased and/or fastened production of provisional matrix, the improved re-epithelialization may be characterized by an increased and/or fastened activation (i.e. proliferation) and/or migration of keratinocytes. In such embodiments, the amount of activated keratinocytes may be increased by at least about 10%, preferably at least about 25%, preferably at least about 50%, preferably at least about 75%, more preferably at least about 100%, and/or the migration of keratinocytes may be increased by at least about 10%, preferably at least about 25%, preferably at least about 50%, preferably at least about 75%, more preferably at least about 100% when compared to a subject that is not treated with the composition (optionally hydrogel) described herein.

In particular embodiments, the compositions and hydrogels of the above aspects are for use increasing proliferation of cells at a wound site of a subject. Proliferation of cells may include without limitation the proliferation of endothelial cells, fibroblasts and/or keratinocytes. In certain embodiments, the proliferation of cells selected from the group of cells consisting of endothelial cells, fibroblasts, keratinocytes, and any combination thereof is increased by at least 10%, preferably at least 25%, more preferably at least 50%, even more preferably by at least 75%, most preferably by at least 100% when compared to the cell proliferation at a wound site in a subject that is not treated with the composition described herein.

In particular embodiments, the composition described herein is used for reducing an inflammatory response in a wound site of a subject. In such embodiment, the composition described herein is used for reducing the amount of one or more pro-inflammatory molecules and/or increasing the amount of one or more anti-inflammatory molecules at a wound site of a subject. Non-limiting examples of pro-inflammatory molecules envisaged herein include without limitation interleukin-1 beta (IL-Iβ), interleukin 4 (IL-4), interleukin 6 (IL-6), interleukin 8 (IL-8), tumor necrosis factor alpha (TNF-α), interferon gamma (IF-γ), interleukin 12 (IL-12), histamine, serotonin, neuropeptides, plasma proteases, complement, kinins, clotting proteins, fibrinolytic proteins, lipid mediators, prostaglandins, leukotrienes, and platelet-activating factor (PAF). In certain embodiments, the composition (optionally a hydrogel) as described herein is used for reducing the amount of one or more pro-inflammatory molecules selected from the group consisting of interleukin-1 beta (IL1-β), interleukin 4 (IL-4), interleukin 6 (IL-6), interleukin 8 (IL-8), tumor necrosis factor alpha (TNF-α), interferon gamma (IF-γ), interleukin 12 (IL-12), histamine, serotonin, neuropeptides, plasma proteases, complement, kinins, clotting proteins, fibrinolytic proteins, lipid mediators, prostaglandins, leukotrienes, and platelet-activating factor (PAF) that is produced at a wound site by at least 10%, preferably by at least 25%, more preferably by at least 50%, vet more preferably by at least 75%, most preferably by about 100% when compared the level of said one or more pro-inflammatory molecules present at a wound site of a subject that is not treated with the composition described herein. Preferably, the medical use or treatment results in an improved macrophage and neutrophil profile indicative for a reduced local wound inflammation versus non-treated wounds. In a particular embodiment, the treatment according to the invention decreases the number of inflammatory cells such as, but not limited to, macrophages and neutrophils in the wound. Thus, in particular embodiments, topical administration of the compositions according to the invention promotes a pro-resolution wound phenotype, with reduced M1 marker expression and increased M2 marker expression. In some embodiments, the number of both innate and acquired immune cells is reduced upon administration of the compositions of the invention. Typically, in further embodiments, beneficial effects on the function of other immune cells like dendritic cells. Langerhans cells and mast cell can be observed when the compositions as described herein are administered.

In preferred embodiments, use of the composition (e.g. a hydrogel) comprising the estetrol component results in improved histological healing parameters when compared to wounds that are not treated with any wound healing composition. More preferably, use of the composition comprising the estetrol component results in improved histological healing parameters when compared to wounds that are not treated with a wound healing composition not comprising the estetrol component. Optionally, the histological healing parameters is a histological skin parameter selected from the group consisting of: epidermal closure, epidermal differentiation, epidermal migration, granulation tissue formation and epidermal hyperplasia, granulation tissue and matrix formation, inflammation, and late stage matrix remodelling, which can be assessed histologically by respectively the presence of a newly formed epidermis, spinous and/or granulous epidermal differentiation markers, migrating cells, proliferating cells, collagen fibre deposition, immune cell markers, wound protease levels and matrix composition. Each of these parameters have been described in detail in the art (e.g. in Gupta and Kumar, Plast Aesthet Res. 2015). Alternative histological parameters that may be derived from one or more observations include but are not limited to the length of the reepithelialisation zone, the distance between the wound borders, the depth of the wound, the width of the wound, the thickness of the connective tissue, and the thickness of the natural dermis on the wound edges, the orientation of dermal matrix, wound cellularity, wound vascularisation.

In preferred embodiments, the composition described herein may be used for improving the incidence of complete wound closure in a subject, which optionally is a subject characterised by impaired wound healing. Complete wound closure indicates that the surface of the skin is fully closed, i.e. fully resurfaced with new epithelium. In such embodiments, the incidence of complete wound closure by using the composition comprising an estetrol component is increased by at least 25%, preferably by at least 50%, more preferably by at least 75%, most preferably by at least 100% when compared to wounds not treated with the composition, or based on the wound healing history of the subject.

In preferred embodiments, the composition described herein may be used for accelerating the time to achieve wound closure of a wound in a subject, which optionally is a subject characterised by impaired wound healing. In such embodiments, the time to achieve wound closure of a wound in a subject is reduced by at least 25%, preferably at least 50%, more preferably at least 75%, most preferably more than 80%, when compared to wounds not treated with the composition, or based on the wound healing history of the subject. A skilled person appreciates that certain parameters described in detail throughout the present disclosure, such as but not limited to a certain time that is needed to achieve full wound closure, is dependent inter alia on the size of the wound.

In preferred embodiments, the composition described herein may be used for facilitating surgical wound closure in a subject, which optionally is a subject characterised by impaired wound healing. In such embodiments, the composition described herein may increase the speed of surgical wound closure and/or increase the chance for a subject to achieve wound closure of wounds having an area and/or depth exceeding the area and/or depth of wounds that could be healed by said subject without use of the composition comprising the estetrol component.

In some embodiments the quality of the healing is improved, particularly in an infected wound.

The terms “formulation” or “composition” may be used interchangeably herein. In any of the embodiments concerning the compositions described herein, it is evident that said compositions may comprise one or more pharmaceutically or cosmetically acceptable carriers (i.e. excipients) that are not described in detail throughout the present disclosure. The term “pharmaceutically acceptable” as used herein is consistent with the art and means compatible with the other ingredients of a pharmaceutical or cosmetic composition and not deleterious to the recipient thereof. In a particularly preferred embodiment of the invention the (pharmaceutical) composition according to invention is designed for daily administration, i.e. it represents a daily dosage unit. The excipients that may be used in the pharmaceutical composition is not particularly limited and may therefore be one or more excipients selected from the group consisting of: an active pharmaceutical ingredient excipients, binder excipients, carrier excipients, co-processed excipients, coating system excipients, controlled release excipients, diluent excipients, disintegrant excipients, dry powder inhalation excipients, effervescent system excipients, emulsifier excipients, lipid excipients, lubricant excipients, modified release excipients, penetration enhancer excipients, permeation enhancer excipients, pH modifier excipients, plasticiser excipients, preservative excipients, preservative excipients, solubilizer excipients, solvent excipients, sustained release excipients, sweetener excipients, taste making excipients, thickener excipients, viscosity modifier excipients, filler excipients, compaction excipients, dry granulation excipients, hot melt extrusion excipients, wet granulation excipients, rapid release agent excipients, increased bioavailability excipients, dispersion excipients, solubility enhancement excipients, stabiliser excipients, capsule filling excipients, or any combination hereof. A skilled person is aware that use of such media and agents for pharmaceutical active substances is common practice and incorporation of these excipients is hence well known in the art. It is evident that all of the used ingredients should be non-toxic in the concentration contained in the final pharmaceutical composition and should not negatively interfere with the activity of the estetrol component, said estetrol component preferably being present in the pharmaceutical composition as the predominant pharmaceutically active ingredient. In certain embodiments, more than one excipient which a skilled person would classify as belonging to the same group of excipients is added to the pharmaceutical composition. In further embodiments, more than one excipient wherein the different excipients belong to different groups is added to the pharmaceutical composition. In certain embodiments, the excipients may fulfil more than one function and/or be classified by a skilled person as belonging to different groups or classes of excipients.

As indicated above, the particulars about the subject affected by a wound is not particularly limiting in the context of the present invention. Preferred subjects are elderly subjects. “Elderly subject” refers to a subject of old age, i.e. the age nearing or surpassing the life expectancy of a subject. An elderly subject is defined by an age of at least 60 years, preferably at least 70, at least 75, at least 80, at least 85, most preferably at least 85 years. In alternative embodiments, the subject is selected from the group consisting of: infants (i.e. juvenile subjects), adolescent subjects, and adult subjects. In certain embodiments described herein, the subject is diagnosed to be palliative or considered to be palliative.

Optionally, the pharmaceutical compositions envisaged by the present invention (which may optionally be hydrogels) may comprise further skin active components capable of providing a skin care benefit. The skin care benefit may include but is not limited to benefits related to cosmetic appearance of the skin. The further skin active component may provide an immediate and short lived (i.e. acute) benefit, and/or a long term and long lasting (i.e. chronic) benefit.

Optionally, the pharmaceutical compositions envisaged by the present invention (which may optionally be hydrogels) may comprise at least one further pharmaceutically active ingredient in addition to the estetrol component. In particular embodiments, the at least one further pharmaceutically active ingredient is selected from the group consisting of: anti-inflammatory agents, analgesic agents, and anti-infective agents. The anti-inflammatory ingredient may be a steroidal anti-inflammatory ingredient, a nonsteroidal anti-inflammatory ingredient, or a combination thereof. The analgesic ingredient (i.e. an ingredient capable of inducing a relief of pain) may be a non-opioid analgesic or an opioid analgesic. Suitable anti-infective agents include but are not limited to antibiotics.

In particular embodiments, the pharmaceutical compositions envisaged by the present invention (which may optionally be hydrogels) are used for wound healing in a context of skin grafting. In such embodiments, the compositions described herein may be combined with skin transplantation techniques such as but not limited to those relying on stem cell therapy, bioengineered skin, skin equivalents, skin substitutes, synthetic skin, or combinations thereof. In particular embodiments the compositions as used herein are used in wound healing after a skin transplantation deemed necessary due to extensive injuries to a considerable area of the skin of a subject, such as but not limited to skin transplantation in a context of burn wounds. Envisaged herein is also the healing of wounds from a skin graft donor site.

The pharmaceutical composition (which optionally is a hydrogel) as described herein is administered (i.e., applied) to the wound site. The composition may be applied by pouring, dropping, spraying, rubbing, or by any other appropriate means. Optionally, the composition is administered once to a wound site. Alternatively, the composition is administered at multiple points in time to a wound site, preferably at substantially regularly interspersed time points. In certain embodiments, the composition is administered daily to a wound site. Optionally, the composition is administered to and maintained on (i.e. not removed from the wound site) for a prolonged period of time corresponding to at least 30 minutes, preferably at least 1 hour, more preferably at least 2 hours, more preferably at least 4 hours, more preferably at least 8 hours, more preferably at least 1 day, more preferably at least 1 week, more preferably at least 1 month. In such embodiments, it is understood that the wound is continuously exposed to the composition for the indicated amount of time.

The terms “continuous”/“continuously” as used herein, means that the components are administered at relatively regular intervals, with no (therapeutically) significant interruptions. Naturally, minor interruptions may occur that do not affect the overall effectiveness of the present method, and indeed such aberrations are encompassed by the present invention.

The pharmaceutical composition (which optionally is a hydrogel) as described herein may be part of, i.e., comprised in any means that is suitable for application to the skin and/or wound site of a subject, preferably on or in close proximity to the wound site to deliver a pharmaceutically active ingredient to the skin (and/or wound site). Alternatively, the composition as described herein may be applied to any means that is suitable for application to the skin and/or wound site of a subject by said subject, another subject, or a skilled medical practitioner prior to application of the composition to the skin or wound site of the subject (i.e. the composition is used in conjunction with the means for application to the skin).

Preferably, the pharmaceutical composition is applied to the skin of the subject by means of a dressing. Numerous types of dressing have been described in the art and include without limitation gauze dressings, tulle dressings, alginate dressings, polyurethane dressings, film dressings, polysaccharide paste dressings, granule dressings, foam dressings, silicone dressings, synthetic polymer scaffold dressings, hydrocolloid dressings, occlusive dressings or combinations thereof. The dressing may be adhesive or non-adhesive. The term “occlusive dressing” as used herein refers to a dressing that prevents air and/or bacteria from contacting which retains one or more of the following: moisture, heat, body fluids, and medication. A skilled person is capable of selecting a suitable wound healing dressing to be used on a particular wound, and said selection may be made in function of parameters such as but not limited to the type of the wound, size of the wound, and healing progression of the wound.

Optionally, the dressing is a hydrogel dressing. Hydrogel dressings are composed to a large extent of water in a network of fibres that maintain integrity of the polymer gel. Water from said dressing is released to preserve an adequate moisture level of the wound. Examples of hydrogel dressings include without limitation Tegagel® and Intrasite®.

Methods and protocols to produce any of the above dressings have been described in the art. The estetrol component may be incorporated into/onto the dressing upon manufacturing of said dressing, but may equally be applied to a premanufactured dressing. By means of illustration and not limitation, a premanufactured dressing or portion thereof may be impregnated with the estetrol component. Alternatively, a premanufactured dressing or portion thereof may be coated with the estetrol component.

In particular embodiments, the pharmaceutical composition (which optionally is a hydrogel) is comprised in a skin replacement (i.e. skin substitute or dermal substitute). A skin substitute provides a three dimensional biomatrix that fulfil the functions of a cutaneous dermal layer that is able to either temporarily or permanently cover open skin wounds. The material of said skin substitute is not particularly limited, and may therefore comprise of biological materials, synthetic material, or combinations thereof. Non-limiting examples of biological material include without limitation human or porcine skin, and human or porcine intestine submucosa. The biological skin substitute may comprise different constituents including but not limited to collagen, glycosaminoglycan, fibronectin, hyaluronic acid, elastine, and any combinations thereof.

Alternatively, the pharmaceutical composition may be comprised in any other suitable means for application to skin tissue of a subject, and may therefore be comprised in means such as but not limited to bandages, band aids, patches, and plasters.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims. The herein disclosed aspects and embodiments of the invention are further supported by the following non-limiting examples.

### EXAMPLES

### Example 1. Manufacturing Processes

### Aqueous Gel Formulations

### Batches AG23, AG24, AG25, AG26

i. Water (first addition) was weighed into a Duran (vessel 1),
ii. The sample from Step (i) was placed on a hot plate stirrer at 800 rpm forming a vortex.
iii. The polymer (Carbopol®) was weighed via a weighing boat and dispersed in the contents of Step (ii) maintaining the vortex. The sample was left on stir for at least an hour to allow polymer dispersion.
iv. The weight of polymer dispersion in Step (iii) was recorded.
v. The sample from Step (iv) was autoclaved at standard conditions (121° C.+2° C., 2×10⁵ Pa, 15 mins).
vi. Once the autoclaved sample was cooled, the sample from Step (v) was re-weighed and additional water was added to compensate volatile loss from evaporation.
vii. The solvents (PEG 400, Transcutol® P (diethylene glycol monoethyl ether)) were weighed into a separate vessel.
viii. Estetrol monohydrate was weighed into the contents of Step (vii) and stirred at 500 rpm for 2.5 hours (hotplate magnetic stirrer at laboratory room temperature) to dissolve the drug.
ix. In the biosafety laminar flow hood, the contents of Step (viii) were filtered through multiple Spartan (regenerated cellulose) 0.2 μm sterile filters into multiple 20 mL autoclaved vials, this was to avoid the contamination of the bulk should a filter break,
x. The contents of Step (ix) were combined and weighed into a pre-autoclaved Duran vessel,
xi. The contents of Step (x) were poured into the contents of Step (vi) and mixed with an overhead stirrer and spatula: the mixing speed and time were recorded in the laboratory specific notebook,
xii. Water (ca. 4% of the total amount) was used to rinse the vessel from Step (x) and poured into vessel 1.
xiii. The formulation was left to rest for at least overnight
xiv. The pH of the sample was adjusted between the target pH, 6-6.5 with sodium hydroxide solution and taken to weight with water.
xv. It is to be noted that manufacturing Steps (ix) to (xiv) were performed under aseptic conditions in a laminar flow hood.

### Batches AG30, AG22 (Carbopol® 980)

i. Water (first addition) was weighed into a 150 mL Duran (vessel 1).
ii. The sample from Step (i) was placed on a hot plate stirrer at 800 rpm forming a vortex.
iii. The polymer (Carbopol® 980) was weighed via weighing boat and dispersed in the contents of Step (ii) maintaining the vortex. The sample was left on stir for at least an hour to allow polymer dispersion.
iv. The weight of polymer dispersion in Step (iii) was recorded,
v. PEG 400 and glycerol was weighed and added to the contents of Step (iv).
vi. The sample from Step (v) was autoclaved at standard conditions (121° C.+2° C., 2×10⁵ Pa, 15 mins).
vii. Once the autoclaved sample cooled, the sample from Step (v) was re-weighed and additional water was added to compensate volatile loss from evaporation.
viii. The solvents (propylene glycol (PG), Transcutol® P) were weighed into a 100 mL Duran,
ix. Estetrol monohydrate was weighed into the contents of Step (viii) and stirred at 500 rpm (hotplate magnetic stirrer at laboratory room temperature).
x. In the biosafety laminar flow hood, the contents of Step (ix) was filtered through multiple Spartan 0.2 μm sterile filters into multiple 20 mL autoclaved vials to avoid the contamination of the bulk should a filter break.
xi. The contents of Step (x) were combined and weighed into a pre-autoclaved 100 mL Duran,
xii. Sodium hydroxide 18% (previously filtered through a 0.2 μm sterile Spartan filter) was poured into the contents of Step (xi) and mixed.
xiii. The contents of Step (xii) was swiftly poured into the contents of Step (vii) and mixed with a spatula.
xiv. Water (ca. 4% of the total amount) was used to rinse the vessel from Step (x) and poured into vessel 1,
xv. The thickened sample was left to rest for at least overnight,
xvi. The pH of the sample was measured, and the remaining sterile water was added, alongside sodium hydroxide solution (also previously sterilised through a 0.2 μm Spartan filter) to adjust to pH 7-7.5 where required.

It is to be noted that manufacturing Steps (x) to (xvi) were performed under aseptic conditions in a laminar flow hood.

### Batch AG27 (Poloxamer)

i. Water (first addition) was weighed into a 150 mL Duran and cooled in a fridge maintained at 2-8° C., for 20 minutes.
ii. The sample from Step (i) was placed on a hot plate stirrer and poloxamers (P407 followed by P188) were weighed and added over a vortex at 800 rpm.
iii. The sample from Step (ii) were stirred with the aid of an overhead stirrer for 6 hours at 200 rpm until the poloxamer dissolved in the water bath maintained at 2-8° C.,
iv. In a separate vessel, PEG 400, benzyl alcohol (BA) and PG were weighed together,
v. Estetrol monohydrate was weighed and added to the contents of Step (iv) and left to stir overnight in a hot plate stirrer at 500 rpm.
vi. Once the drug dissolved, the required amount of solution of Step (v) was weighed onto the first vessel containing the poloxamer solution. The sample was left on a magnetic stirrer overnight at 300 rpm.
vii. The sample from Step (vi) was pH adjusted to 7-7.5.

### Batches AG28 (Hydroxyethyl Cellulose (HEC)), AG29 (Carboxymethyl Cellulose (CMC))

i. The solvent system for the formulation was prepared by weighing out all solvents, except water/buffer for the active systems only.
ii. The drug was added to the solvent systems for the active formulations and put on stir at 500 rpm overnight.
iii. Following the dissolution of the drug, whilst the Duran is on stir, the water/pH 7.0 buffer solution was added to the solvent system,
iv. A suitable amount of premix was weighed into a separate 150 mL Duran.
v. The polymer was dispersed whilst forming a vortex stirring at 800 rpm and the formulation was left to stir overnight at 500 rpm.
vi. For AG28, the pH was adjusted to pH 7-7.5, pH adjustment was not required for AG29 as it contains buffer phosphate-phosphate pH 7.0.

### Batch AG15 (Poloxamer)

i. Water (first addition) was weighed into a 150 mL Duran and cooled in a fridge maintained at 2-8° C., for 20 minutes.
ii. The sample from Step (i) was placed on a hot plate stirrer and poloxamers (P407 followed by P188) were weighed and added over a vortex at 800 rpm.
iii. The sample from Step (ii) was stirred with the aid of an overhead stirrer for 6 hours at 200 rpm until the poloxamer dissolved in the water bath maintained at 2-8° C.,
iv. In a separate vessel, PEG400, BA and PG were weighed together.
v. Estetrol was weighed and added to the contents of Step (iv) and left to stir overnight in a hot plate stirrer at 500 rpm. This step was omitted for the manufacture of placebo.
vi. Once the drug dissolved, the required amount of solution of Step (v) was weighed onto the first vessel containing the poloxamer solution. The sample was left on a magnetic stirrer overnight at 300 rpm.
vii. The sample from Step (vi) was pH adjusted to 7-7.5.

### Batch AG17

i. Water (first addition) was weighed into a 150 mL Duran (vessel 1).
ii. The sample from Step (i) was placed on a hot plate stirrer at 800 rpm forming a vortex.
iii. The polymer (Carbopol®) was weighed via weighing boat and dispersed in the contents of Step (ii) maintaining the vortex. The sample was left on stir for at least an hour to allow polymer dispersion.
iv. The weight of polymer dispersion in Step (iii) was recorded.
v. The sample from Step (iv) was autoclaved at standard conditions (121° C.±2° C., 2×10⁵ Pa, 15 mins).
vi. Once the autoclaved sample cooled, the sample from Step (v) was re-weighed and additional water was added to compensate volatile loss from evaporation.
vii. Benzyl alcohol, PEG 400 and PG were weighed into a separate 100 mL Duran.
viii. Estetrol monohydrate was weighed into the contents of Step (vii) and stirred at 500 rpm overnight (hotplate magnetic stirrer at laboratory room temperature) to dissolve the drug. This step was omitted for the placebo formulation
ix. Sodium hydroxide 18% (1.38 g) was poured into the contents of Step (vi) and mixed.
x. The contents of Step (ix) were swiftly poured into the contents of Step (vi) and mixed with a spatula,
xi. Water (4.5 g) was used to rinse the vessel from Step (ix) and poured into vessel 1,
xii. The sample which had thickened was left to rest for at least overnight.
xiii. The pH of the sample was measured, and the remaining water was added gradually whilst continuing to measure the pH.

### Batches AG18 (HEC), AG19 (CMC)

i. The solvent systems for placebo and active formulations were prepared by weighing out all solvents, except water/buffer for the active systems only.
ii. The drug was added to the solvent systems for the active formulations and put on stir at 500 rpm overnight.
iii. Following the dissolution of the drug, whilst the Duran was on stir, the water/pH 7.0 buffer solution was added to the solvent system.
iv. A suitable amount of premix was weighed into a separate 150 mL Duran.

The polymer was dispersed whilst forming a vortex stirring at 800 rpm and the formulation was left to stir overnight at 500 rpm.
v. For AG18, the pH was adjusted to pH 7-7.5, pH adjustment was not required for AG19 as it contained buffer phosphate-phosphate pH 7.0.

### Batches AG20, AG23

i. Steps (i)-(vi) from AG17 were followed.
ii. The solvents (PEG 400, Transcutol® P) were weighed into a separate vessel (100 mL).
iii. Estetrol monohydrate was weighed into the contents of Step (ii) and stirred at 500 rpm for 2.5 hours (hotplate magnetic stirrer at laboratory room temperature) to dissolve the drug. This step was omitted for the placebo formulation.
iv. In the biosafety laminar flow hood, the contents of Step (iii) were filtered through multiple Spartan (regenerated cellulose) 0.2 μm sterile filters into multiple 20 mL autoclaved vials, this was to avoid the contamination of the bulk should a filter break. It should be noted that initially the samples were going to be filtered through the Nalgene bottle system filter (PES), with the aid of a vacuum pump but the filters broke, hence Spartan syringe filters were used.
v. The contents of Step (iv) were combined and weighed into a pre-autoclaved 100 mL Duran.
vi. The contents of Step (v) were poured into the contents of Step (i) and mixed with a spatula.
vii. Water (4.5 g) was used to rinse the vessel from Step (v) and poured into vessel 1.
viii. As the formulation did not thicken, it was left to rest for at least overnight.
ix. The pH of the sample was adjusted between 6-6.5 with sodium hydroxide solution and taken to weight with water. It is to be noted that initially a process with sodium hydroxide 18% was performed, however, Carbopol® clumped or failed to hydrate. Furthermore, the original target pH was 7-7.5, however, precipitation of the polymer was observed above pH 6.5, samples were repeated and left between pH 6-6.5 to avoid this issue.

It is to be noted that manufacturing Steps (iv) to (ix) were performed under aseptic conditions in a laminar flow hood.

### Batches AG21, AG22

i. Steps (i) to (vi) from AG17 were followed.
ii. The solvents (PEG 400, PG, Trans P) were weighed into a 100 ml Duran.
iii. Estetrol monohydrate was weighed into the contents of Step (i) and stirred at 500 rpm (hotplate magnetic stirrer at laboratory room temperature). The drug dissolved within 2.5 hours of stirring for AG22 and overnight for AG21. This step was omitted for the placebo formulations.
iv. In the biosafety laminar flow hood, the contents of Step (viii) were filtered through multiple Spartan 0.2 μm sterile filters into multiple 20 mL autoclaved vials to avoid the contamination of the bulk should a filter break.
v. The contents of Step (ix) were combined and weighed into a pre-autoclaved 100 mL Duran.
vi. Sodium hydroxide 18% (1.38 g) (previously filtered through a 0.2 μm sterile Spartan filter) was poured into the contents of Step (x) and mixed.
vii. The contents of Step (xi) were swiftly poured into the contents of Step (vi) and mixed with a spatula,
viii. Water (4.5 g) was used to rinse the vessel from Step (x) and poured into vessel 1.
ix. The sample thickened and was left to rest for at least overnight.
x. The pH of the sample was measured, and the remaining sterile water was added, along sodium hydroxide solution (also previously sterilised through a 0.2 μm Spartan filter) to adjust to pH 7-7.5 where required.

It is to be noted that manufacturing Steps (ix) to (xv) were performed under aseptic conditions in a laminar flow hood.

TABLE 1
Compositions (% w/w) of estetrol monohydrate (E4) aqueous gel formulations.

| Formulation | AG15 | AG17 | AG18 | AG19 | AG20 | AG21 | AG22 | AG23 |
|---|---|---|---|---|---|---|---|---|
| Estetrol | 0.24 | 0.5 | 0.5 | 0.5 | 0.44 | 0.37 | 0.06 | 0.5 |
| monohydrate | | | | | | | | |
| (E4) | | | | | | | | |
| Buffer | — | — | — | 48.00 | — | — | — | — |
| phosphate- | | | | | | | | |
| phosphate | | | | | | | | |
| pH 7.0 | | | | | | | | |
| PEG 400 | 14.18 | 18.00 | 18.00 | 18.00 | 45.00 | 35.00 | 10.00 | 50.00 |
| Propylene | 14.18 | 20.00 | 20.00 | 20.00 | — | 5.00 | — | — |
| glycol | | | | | | | | |
| Transcutol ® P | — | — | — | — | — | — | 5.00 | 5.00 |
| Glycerol | — | 10.00 | 10.00 | 10.00 | — | 5.00 | — | — |
| Benzyl alcohol | 2.00 | 2.00 | 2.00 | 2.00 | — | — | — | — |
| Water (1st | 45.00 | 40.00 | 40.00 | — | 45.00 | 45.00 | 75.00 | 35.00 |
| Addition) | | | | | | | | |
| Carbopol ® 980 | — | 0.50 | — | — | 0.50 | 0.50 | 0.50 | 0.50 |
| HEC 250 HHX | — | — | 1.50 | — | — | — | — | — |
| CMC | — | — | — | 1.50 | — | — | — | — |
| Poloxamer 188 | 3.75 | — | — | — | — | — | — | — |
| Poloxamer 407 | 15.00 | — | — | — | — | — | — | — |
| Sodium | — | Adjust to | | — | Adjust to | | | Adjust to |
| hydroxide | | pH 7-7.5 | | | pH 7-7.5-7.5 | | | pH 6-6.5 |
| Citric | To | — | — | — | — | — | — | — |
| acid 0.1M* | adjust | | | | | | | |
| | to pH | | | | | | | |
| | 7-7.5 | | | | | | | |
| Water (2^{nd} | q.s. 100% | | | — | q.s. 100% | | | |
| addition) | | | | | | | | |
| TOTAL | | | | | | | | |

| Formulation | AG24 | AG25 | AG26 | AG27 | AG28 | AG29 | AG30 |
|---|---|---|---|---|---|---|---|
| Estetrol | 0.50 | 0.22 | 0.06 | 0.06 | 0.06 | 0.06 | 0.06 |
| monohydrate | | | | | | | |
| (E4) | | | | | | | |
| Buffer | — | — | — | — | — | 48.44 | — |
| phosphate- | | | | | | | |
| phosphate | | | | | | | |
| pH 7.0 | | | | | | | |
| PEG 400 | 43.95 | 33.05 | 20 | 14.18 | 18.00 | 18.00 | 35.00 |
| Propylene | — | — | — | 14.18 | 20.00 | 20.00 | 5.00 |
| glycol | | | | | | | |
| Transcutol ® P | 1.00 | 1.00 | 5.00 | — | — | — | — |
| Glycerol | — | — | — | — | 10.00 | 10.00 | 5.00 |
| Benzyl alcohol | — | — | — | 2.00 | 2.00 | 2.00 | — |
| Water (1st | 43.24 | 55.45 | 63.27 | 45.00 | 40.00 | — | 45.00 |
| Addition) | | | | | | | |
| Carbopol ® 980 | 0.50 | 0.50 | 0.50 | — | — | — | 0.50 |
| HEC 250 HHX | — | — | — | — | 1.50 | — | — |
| CMC | — | — | — | — | — | 1.50 | — |
| Poloxamer 188 | — | — | — | 3.75 | — | — | — |
| Poloxamer 407 | — | — | — | 15.00 | — | — | — |
| Sodium | Adjust to | | | Adjust to | | — | Adjust |
| hydroxide | pH 6-6.5 | | | pH 7-7.5 | | | pH 7-7.5 |
| Citric | — | — | — | — | — | — | — |
| acid 0.1M* | | | | | | | |
| Water (2^{nd} | q.s. 100% | | | | | — | q.s. |
| addition) | | | | | | | to 100% |
| TOTAL | | | | | | | |

### Creams

### Batches CR01, CR05, CR12

i. Placebo and active solvent systems were prepared by weighing out all solvents, except water/buffer for the active systems only.
ii. The drug was added to the solvent systems for the active creams and put on stir at 500 rpm overnight.
iii. After the drug had completely dissolved, whilst the container (Duran) was on stir, the water/pH 7.0 buffer solution was added to the solvent system.
iv. The oil phases were then prepared. For CR01, the oil phase was placed in the water bath at 75° C., until it was melted.
v. For CR05 and CR12, the oil phases were put in the oven for 2 hours at 160° C. (to mimic the process of sterilisation). Once the oil phases had cooled to room temperature, and re-solidified, they were placed in the water bath (75° C.) until the oil phases melted again.
vi. Before processing the active and placebo creams, the required amount of the solvent system pre-mix was weighed out into a separate Duran.
vii. The Durans with the pre-mix was placed in the water bath along with the homogeniser head for 5 minutes, viii. For CR01 and CR12, the creams were processed via an Ultra-turrax equipped with a 25G dispersing head at 10,000 rpm for 2 minutes.
ix. For CR05, during homogenisation, the cream was homogenised at 5000 rpm, for 5 minutes instead, to avoid overflowing.
x. After processing, the creams were hand stirred with a metal spatula until the cream reached room temperature,
xi. They were left to cure overnight,
xii. Formulations were pH adjusted to pH 7-7.5.

### Batches CR10, CR13, CR14, CR15, CR16

i. Placebo and active solvent systems were prepared by weighing out all solvents, except water/buffer for the active systems only.
ii. The drug was added to the solvent systems for the active creams and put on stir at 500 rpm overnight,
iii. After the drug had completely dissolved, whilst the Duran was on stir, the water/pH 7.0 buffer solution was added to the solvent system.

The oil phases of CR13, CR14 and CR15 were put in the oven for 2 hours at 160° C. (sterilised) and left to re-solidify at room temperature.
v. The aqueous phases were sterilised by filtration using sterile PES syringe filters 0.2 μm.
vi. The required amount of aqueous phase was weighed into a 250 mL Duran which was pre-sterilised by autoclaving.
vii. The oil phases were placed in a water bath at 75° C., until molten.
viii. Aqueous phases placed in the water bath along with the homogeniser head to equilibrate for 5 minutes.
ix. For each cream, oil phase was added to the respective aqueous phase and homogenised using an Ultra-turrax equipped with a 25G dispersing head at 10,000 rpm for 2 minutes.
x. After processing, the creams were hand stirred with a metal spatula until the cream reached room temperature,
xi. They were left to cure overnight.
xii. Formulations were pH adjusted to pH 7-7.5

It is to be noted that manufacturing Step (v) to (xii) were performed under aseptic conditions in a laminar flow hood and the sterile vessels were only opened in sterile laminar flow hood.

TABLE 2
Compositions (% w/w) of estetrol monohydrate (E4) cream formulations.

| Formulation | CR01 | CR05 | CR07 | CR08 | CR09 | CR10 | CR11 | CR12 | CR13 | CR14 | CR15 | CR16 |
|---|---|---|---|---|---|---|---|---|---|---|---|---|
| Estetrol | 0.24 | 0.24 | 0.28 | 0.21 | 0.35 | 0.50 | 0.06 | 0.28 | 0.21 | 0.35 | 0.06 | 0.50 |
| monohydrate | | | | | | | | | | | | |
| (E4) | | | | | | | | | | | | |
| Water | 49.76 | 49.76 | 39.72 | 44.79 | 39.65 | — | 64.94 | 29.72 | 34.79 | 29.65 | 54.94 | 34.50 |
| Buffer | — | — | — | — | — | 39.50 | — | — | — | — | — | — |
| phosphate- | | | | | | | | | | | | |
| phosphate | | | | | | | | | | | | |
| pH 7.0 | | | | | | | | | | | | |
| PEG 400 | 14.00 | 14.00 | — | 25.00 | 30.00 | 40.00 | 10.00 | — | 25.00 | 30.00 | 10.00 | 40.00 |
| Propylene | 14.00 | 14.00 | 28.00 | — | — | — | — | 28.00 | — | — | — | — |
| glycol | | | | | | | | | | | | |
| Transcutol ® P | — | — | — | — | 5.00 | — | 5.00 | — | — | 5.00 | 5.00 | — |
| Glycerol | — | — | 10.00 | 10.00 | 5.00 | — | — | 10.00 | 10.00 | 5.00 | — | — |
| Benzyl alcohol | 2.00 | 2.00 | 2.00 | — | — | — | — | 2.00 | — | — | — | — |
| Polawax ® NF | — | — | — | — | — | 7.00 | — | — | — | — | 7.00 | — |
| Glycerol | — | 1.31 | — | — | — | — | — | 1.31 | 1.31 | 1.31 | 1.31 | — |
| Monostearate | | | | | | | | | | | | |
| Brij S2 | 1.02 | — | 1.02 | 1.02 | 1.02 | 0.57 | 1.02 | — | — | — | — | 0.57 |
| Brij S721 | 2.98 | — | 2.98 | 2.98 | 2.98 | 3.43 | 2.98 | — | — | — | — | 3.43 |
| Cetomacrogol ® | — | 2.69 | — | — | — | — | — | 2.69 | 2.69 | 2.69 | 2.69 | — |
| 1000 | | | | | | | | | | | | |
| Cetyl alcohol | 4.00 | — | 4.00 | 4.00 | 4.00 | — | 4.00 | — | — | — | — | — |
| Stearic acid | 4.00 | — | 4.00 | 4.00 | 4.00 | — | 4.00 | — | — | — | — | — |
| Cetostearyl | — | 5.00 | — | — | — | — | — | 5.00 | 5.00 | 5.00 | 5.00 | — |
| alcohol | | | | | | | | | | | | |
| Castor oil | — | — | — | — | — | 9.00 | — | — | — | — | — | 9.00 |
| Mineral oil | 8.00 | 11.00 | 8.00 | 8.00 | 8.00 | — | 8.00 | 11.00 | 11.00 | 11.00 | 11.00 | — |
| Water (2nd | q.s. to 100 | | | | | | | | | | | |
| addition) | | | | | | | | | | | | |
| Sodium | Adjust to pH 7.0-7.5 | | | | | | | | | | | |
| hydroxide | | | | | | | | | | | | |
| solution | | | | | | | | | | | | |
| Total | 100.00 | 100.00 | 100.00 | 100.00 | 100.00 | 100.00 | 100.00 | 100.00 | 100.00 | 100.00 | 100.00 | 100.00 |

### Example 2. In Vitro Release Experiment 1

Following method development and a small scale preliminary in vitro release testing (IVRT) experiment, a full scale IVRT experiment was performed using 10 test formulations described in Example 1. The experimental conditions utilized are shown in Table 3.

TABLE 3
Experimental conditions for the full-scale IVRT.

| Parameter | Experimental conditions | |
|---|---|---|
| No. of formulations | 10 | |
| Replicates | n = 6 active; n = 1 blank | |
| Membrane | isopore | |
| Receptor solution | 40:30:30 v/v/v | |
| | Ethanol:PEG400:Water | |
| RS Sampling volume | 1.0 | mL |
| Dose Amount | 0.3 | g |
| Stir rate | 50 | rpm |

| | |
|---|---|
| Sampling Timepoints | t = 0, 30, 60, 120, 180, 240, |
| | 300, 360, 420, 480 minutes |
| Membrane temperature | 32° ± 1° C. |

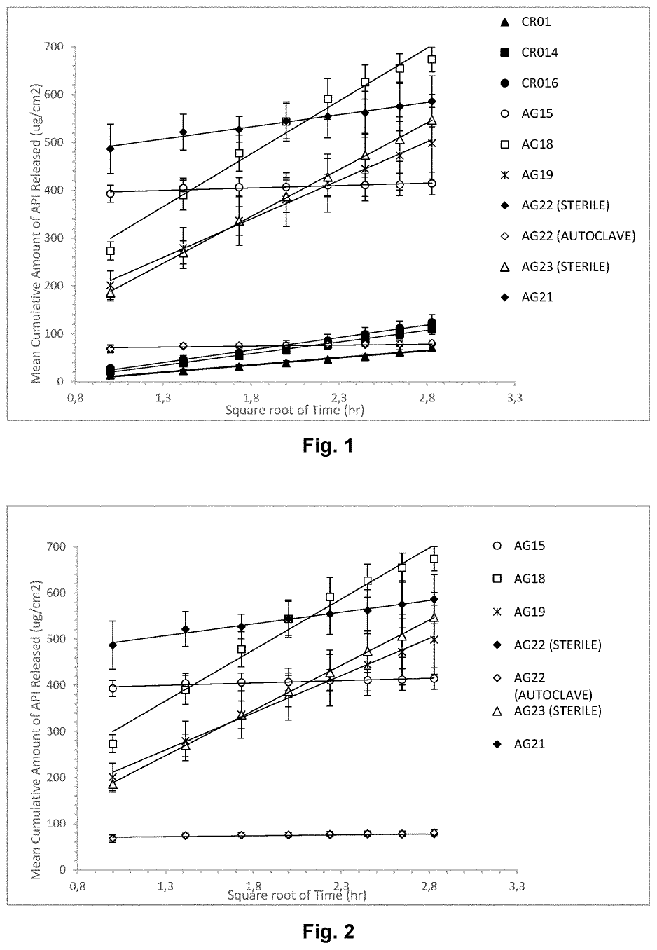

The full scale in vitro release experiment was performed using the experimental conditions developed during the method development and feasibility experiments. The results of all tested gel and cream formulations are shown in FIG. 1, with tabulated results shown in Table 4.

### Aqueous Gel Formulations

The release rates of the aqueous gel formulations are presented in FIG. 2 and table 4. The AG18, AG19, and AG23 formulations resulted in the highest release rates (161-221 μg/cm²/√hr), followed by AG21 (51 μg/cm²/√hr), with AG22 (sterile), AG22 (autoclave), and AG15 resulting in the lowest release rates (3-10 μg/cm²/√hr). As a general trend, the release rates observed correlate with the concentration of API, with the formulations with the highest release rates (AG18, AG19, and AG23) also containing the largest concentration of estetrol monohydrate (0.50% w/w).

The aqueous gels with the lowest release rates (AG21, AG22 (sterile), AG22 (autoclave), and AG15) were found to immediately release a large amount of the dosed API (ca. 50% or greater release at t=0.5 hr) into receptor solution. As a result of the large release of API, linear steady state drug release was not achieved (r2<0.9) for many of the formulations. Therefore, conclusions and statistical comparisons were not performed using the aqueous gel formulations.

### Cream Formulations

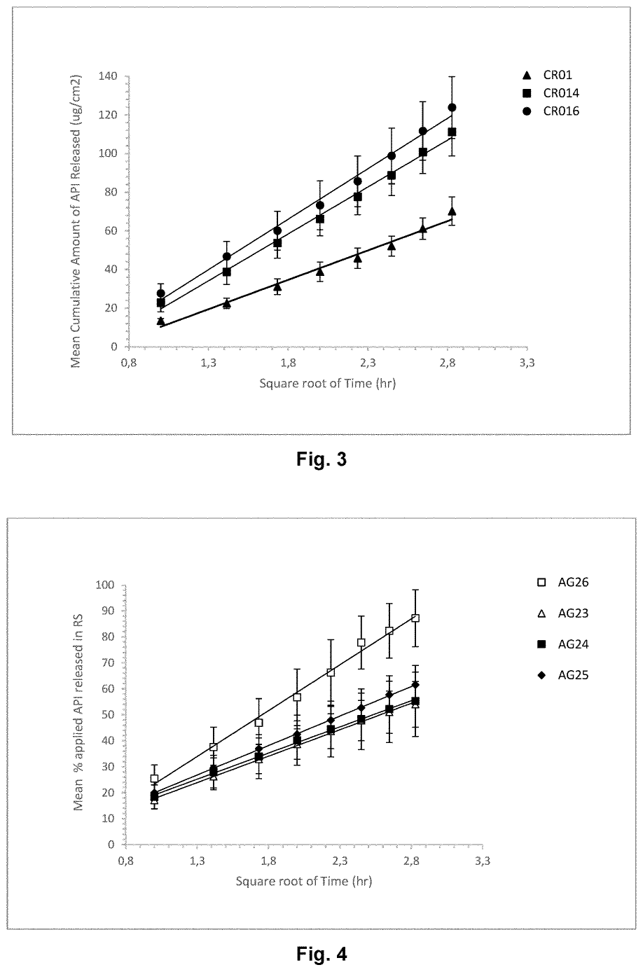

The release rates of the cream formulations are presented in FIG. 3 and complemented by table 4. CR16 (0.50% w/w) resulted in the highest release rate, followed by CR14 (0.35% w/w), and finally CR01 (0.24% w/w), with average release rates ranging from 32-53 μg/cm²/√hr. Following the same trend as the aqueous gels, the release rates correlated with the concentration of API present in the formulation, with the highest release rate observed in the formulation with the highest drug loading (CR16, containing 0.50% API and resulting in a release rate of 52.20 μg/cm²/√hr). One-way statistical analysis of the mean drug release (μg/cm²/√hr) of the cream formulations was performed using Tukey-Kramer (n=6). There were no statistical differences between the release rates of CR14 and CR16 (p>0.05), whereas the release rate of CR01 was observed to have a significantly lower release rate (p<0.05).

TABLE 4
Mean estetrol monohydrate release rate (μg/cm²/√hr)
between the 1 and 8 hr experimental timepoints from
10 formulations across an Isopore ® membrane
in receptor solution 40:30:30 v/v/v ethanol:PEG400:water.

| API Release Rate (slope; μg/cm²/√hr) | | | | Linearity (r2) | | |
|---|---|---|---|---|---|---|
| Formulation | n | Mean | Std Dev | n | Mean | Std Dev |
| AG22 | 6 | 3.85 | 2.33 | 6 | 0.74 | 0.09 |
| (Sterile) | | | | | | |
| AG22 | 6 | 5.54 | 4.36 | 6 | 0.77 | 0.13 |
| (autoclave) | | | | | | |
| AG15 | 6 | 9.97 | 4.75 | 6 | 0.75 | 0.25 |
| AG21 | 6 | 50.75 | 27.27 | 6 | 0.90 | 0.06 |
| AG19 | 6 | 161.01 | 24.52 | 6 | 1.00 | 0.00 |
| AG23 | 6 | 195.69 | 20.22 | 6 | 1.00 | 0.00 |
| (sterile) | | | | | | |
| AG18 | 6 | 220.93 | 6.96 | 6 | 0.98 | 0.02 |
| CR01 | 6 | 30.41 | 3.41 | 6 | 0.98 | 0.02 |
| CR14 | 6 | 48.51 | 4.18 | 6 | 0.99 | 0.00 |
| CR16 | 6 | 52.20 | 6.34 | 6 | 0.99 | 0.01 |

### Example 3. In Vitro Release Experiment 2

Following the results of the first in vitro release experiment (Example 2), a second IVRT experiment was performed using the 4 formulations and experimental parameters outlined in Table 5.

TABLE 5
IVRT study parameters and formulations employed
in in vitro drug release experiment 2.

| IVRT Parameters | Experimental conditions | |
|---|---|---|
| No. of formulations | Four: | |
| | AG23 (0.50% ACT) | |
| | AG24 (0.50% ACT) | |
| | AG25 (0.22% ACT) | |
| | AG26 (0.06% ACT) | |
| Replicates | n = 6 per active | |
| Membrane | Isopore ® | |
| Receptor solution | 40:30:30 v/v/v | |
| | Ethanol:PEG400:water | |
| RS Sampling volume | 1.0 | mL |
| Dose amount | 0.3 | g |
| Stir rate | 50 | rpm |

| | |
|---|---|
| Sampling Timepoints | 0, 30, 60, 120, 180, 240, |
| | 300, 360, 420 and 480 min |
| Membrane temperature | 32 ± 1° C. |

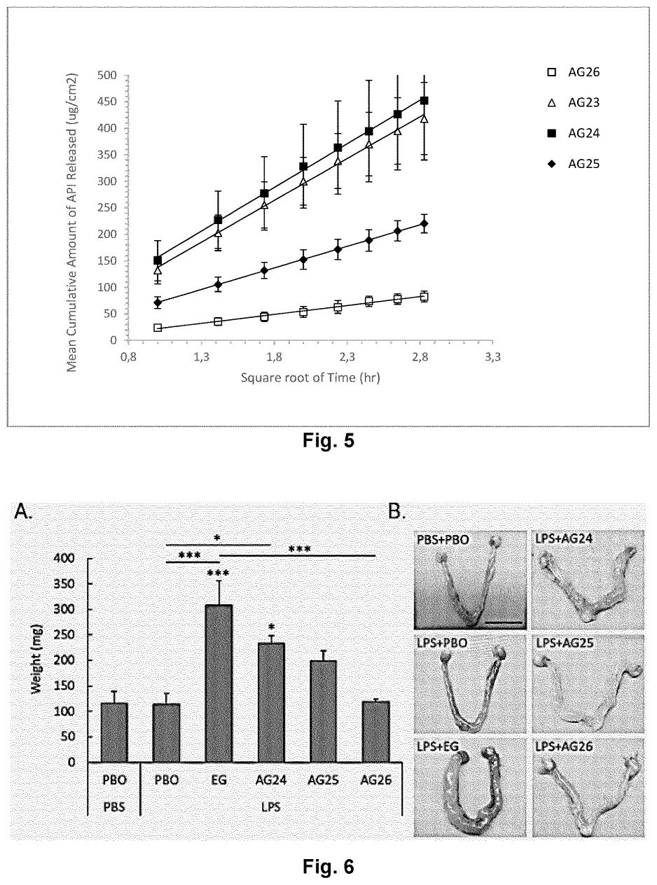

The second full scale in vitro release experiment was performed using the experimental parameters detailed in Table 5 and four aqueous gel formulations. The results of all tested formulations are shown in FIG. 4 and FIG. 5, with tabulated results shown in Table 6 and Table 7.

The purpose of this part of the study was to determine the effect of thermodynamic activity on the rate of release of the estetrol monohydrate from the formulations by comparing a non-optimised formulation (AG23 0.5% (w/w) API) to optimised formulations at a variety of strengths (AG24 0.5% (w/w), AG25 0.22% (w/w) and AG26 0.06% (w/w) API).

When considering these 4 formulations in terms of percentage release of applied dose of API (Table 17), 87.95±10.88% of API was released from formulation AG26 (0.06% (w/w) API) after 8 h, which was significantly higher (p<0.05) than from the other 3 formulations which were not statistically different to each other (p>0.05) (61.51±4.91% AG25 (0.22% (w/w) API), 60.67±4.86% AG24 (0.5% (w/w) API) and 56.88±5.90% AG23 (0.5% (w/w) API)).

Considering the percentage of applied dose release rates over time (slope) there was a significant difference (p<0.05) between formulation AG26 and the other three formulations (AG23, AG24 and AG25). No statistically significant differences were found between AG23, AG24 and AG25. Statistical analysis was performed by Tukey-Kramer test (n=5-6).

During the current study, formulation AG23 displayed a release rate of ca. 167±13 μg/cm²/hr compared to 195±20 μg/cm²/hr in the first IVRT full scale experiment. Formulation AG24 had the highest release rate (ca. 182±8 μg/cm²/hr) in the current study, followed by AG23, AG25 (ca. 81±5 μg/cm²/hr) and finally AG26 (ca. 34±3 μg/cm²/hr). The release rate of each formulation is significantly different (p<0.05) to the next, which in the case of AG23 and AG24, which both contain 0.5% (w/w) estetrol monohydrate, could be due to the issues encountered dosing AG23 or batch to batch variability compared to the batch of AG23 used in the previous experiment (which had a release rate much closer to the one seen here for AG24), or simply a formulation difference between AG23 and AG24. For the other two formulations the difference in release rate is as expected due to the lower API content in these formulations (AG23 and AG24 0.5% w/w, AG25 0.22% (w/w) and AG26 0.06% w/w).

To assess the differences between the aqueous gel formulations as reported above, a one-way statistical analysis was performed using Tukey-Kramer (n=5-6).

TABLE 6
Mean estetrol monohydrate release rate (μg/cm²/√hr)
between the 1 and 8 hr experimental timepoints from
aqueous gel formulations across an isopore membrane
in receptor solution 40:30:30 v/v/v ethanol:PEG400:water.

| API Release Rate (slope; ug/cm²/√hr) | | | | Linearity (r2) | | |
|---|---|---|---|---|---|---|
| Formulation | n | Mean | Std Dev | n | Mean | Std Dev |
| AG24 (0.50%) | 5* | 182.02 | 7.69 | 5 | 1.00 | 0.01 |
| AG23 (0.50%) | 5* | 167.19 | 13.43 | 5 | 1.00 | 0.00 |
| AG25 (0.22%) | 6 | 81.32 | 5.13 | 6 | 1.00 | 0.00 |
| AG26 (0.06%) | 6 | 33.61 | 3.05 | 6 | 0.98 | 0.02 |
| AG23** | 6 | 195.69 | 20.22 | 6 | 1.00 | 0.00 |

*Single replicate removed as identified as an outlier by Dixon's Q test
**Data from full scale IVRT experiment 1

TABLE 7
Cumulative esterol monohydrate (ug/cm2) and percentage of
applied dose released at the 8 hr experimental timepoint
from aqueous gel formulations across an isopore membrane
in receptor solution 40:30:30 v/v/v ethanol:PEG400:water.

| Cumulative Amount of API | | | | | Percentage of applied | |
|---|---|---|---|---|---|---|
| Released (ug/cm²) | | | | | API released | |
| Formulation | n | Mean | Std Dev | n | Mean | Std Dev |
| AG24 | 5* | 495.91 | 39.72 | 5 | 60.67% | 4.86% |
| AG23 | 5* | 440.72 | 45.69 | 5 | 56.88% | 5.90% |
| AG25 | 6 | 220.43 | 17.58 | 6 | 61.51% | 4.91% |
| AG26 | 6 | 82.95 | 10.36 | 6 | 87.16% | 10.88% |

*Single replicate removed as identified as an outlier by Dixon's Q test

### Example 4. In Vivo LPS Treatment Protocol (Delayed Wound Healing Model)

The in vivo LPS-induced delayed wound healing model was reproduced as previously described (Crompton R, Williams H, Ansell D, Campbell L, Holden K, Cruickshank S, Hardman M J. Oestrogen promotes healing in a bacterial LPS model of delayed cutaneous wound repair. Lab Invest. 2016 April, 96 (4): 439-49).

Female wild-type (C57BL/6J) 8 weeks old mice were split into six groups (6 animals per experimental group):
Double placebo control (no LPS, placebo treatment),
LPS control (LPS, placebo treatment),
LPS with topical EstroGel (estradiol gel, contains 0.06% 17β-estradiol as hemihydrate in a hydro-alcoholic gel),
LPS with topical AG24 (E4 at 0.5%),
LPS with topical AG25 (E4 at 0.22%),
LPS with topical AG26 (E4 at 0.06%).

The day prior to wounding (day −1), all the animals were weighed and anaesthetised using oxygen and isoflurane (flow rate 1.25-2 L with 2-2.5% isoflurane depending on clinical signs). Animals were shaved and dorsal areas prepped. Wound positions (2 wounds) were marked on the dorsum of each animal. Animals received a first subcutaneous injection of 2 μg K. pneumoniae-derived LPS diluted in DPBS (1 μg per wound: Sigma Aldrich, UK: L4268) at the wound site, except the double placebo group that received DPBS only. A thin layer of EstroGel, AG24, AG25, AG26 or placebo were then applied to the dorsum of each animal, measured as 60 μl total (30 μl/wound). Animals recovered in a warming cabinet and were then single housed in fresh cages containing alpha pad, RO water, food, mash and a house. On day 0 (24 hours after first anaesthesia), mice were re-anaesthetised and dorsal skin cleaned with chlorhexidine wipes. Two 6 mm dorsal excisions were created. 2 μg LPS (1 μg per wound) was subcutaneously injected at the wound site as above, except for the double placebo mice that were injected with DPBS. EstroGel, AG24, AG25, AG26 or AG23 as Placebo was then applied in a thin layer over the top of the wound (60 μl total: 30 μl/wound), being careful not to damage the “LPS bleb”. Buprenorphine (0.1 mg/Kg), an analgesic, was administered post-operatively via subcutaneous injection in the scruff and each animal was imaged. Mice were recovered in a warming cabinet and returned to single housing. Observations were performed post-operatively.

On days 1, 2, 3 and 4, for treated groups, topical EstroGel, AG24, AG25, AG26 or placebo were re-applied in a thin layer over the top of the wounds (60 μl total: 30 μl/wound) during observations.

On day 5, mice were humanely culled via rising concentration of CO2 and cervical dislocation.

The uteri were removed and weighed.

Non-wounded skin (NS) from the treatment site was collected at the time of wounding (day 0), processed for histological analysis and snap frozen. Wound tissue was collected five days after wounding (day 5). Wounds were halved, with the bottom half of each wound fixed for histological analysis and the top half snap frozen.

To perform immunohistochemistry (IHC) and histological analysis, tissue samples were fixed in 10% buffered formalin and embedded in paraffin wax for sectioning. Tissue sections were dewaxed in xylene and rehydrated through an ethanol gradient before carrying out Haematoxylin and Eosin staining, and IHC for immune cells.

For the isolation and culture of murine peritoneal macrophages, the peritoneal cavity of euthanised mice were filled with 5 ml ice cold PBS supplemented with 3% FBS and 1% antibiotic-antimycotic. The fluid containing the cells was removed using a needle and syringe and cells were seeded into 12-well plates at 1×10⁶ cells/ml in RPM1 growth medium supplemented with 10% FBS and 1% penicillin/streptomycin. Cells were left overnight to allow macrophages to adhere and then washed twice to remove any non-adherent cells. Cells were cultured for a further 24 hours and then polarised to an M1 or M2 state. M1 macrophages were induced using 100 ng/ml IFN-γ and 1 μg/ml LPS. For M2 macrophages. 20 μg/ml anti-IFN-γ and 10 ng/ml IL4 was used.

For isolation of RNA and quantitative real time PCR murine macrophages were collected in Trizol and RNA was isolated using a Trizol plus RNA isolation kit (Invitrogen. Thermo Fisher) according to the manufacturer's instructions. RNA was reverse transcribed to cDNA using GoScript reverse transcriptase (Promega). Quantitative real-time PCR was carried out using 2×Takyon SYBR Green mastermix and a CFX Connect thermocycler. To assess the extent of M1 and M2 polarisation of peritoneal macrophages from the different treatment groups, primers for mouse genes Il-1B, Tnf-α, iNos, Arg1, Fizz1 and Ym1 were used. Primers for GAPDH were used to normalise data. Relative gene expression was set against the M0 PBS+PBO control group unless otherwise stated.

Statistically significance was evaluated using one-way ANOVA with Tukey post hoc analysis or paired t-test as appropriate.

It is apparent from FIG. 6 that EstroGel® application promoted uterine weight gain and hypertrophic changes. Topical application of E4 affects uterine weight in a dose-dependent manner, with AG26 showing no effect and AG24 inducing uterine weight gain. The data confirm that topical application of low concentration E4 formulations (AG26) to open wounds for 6 days, in contrast to EstroGel® (E2) and high-level E4 formulations (AG24, AG25), did not result in systemic side effects. These are expressed in the increase in uterus weight. Next, we evaluated wound closure from histological samples. Histological sections were subjected to K14 immunohistochemistry to visualise the newly forming epidermis. The degree of re-epithelialisation was determined as the length of neo-epidermis divided by the distance between the wound margins, multiplied by 100.

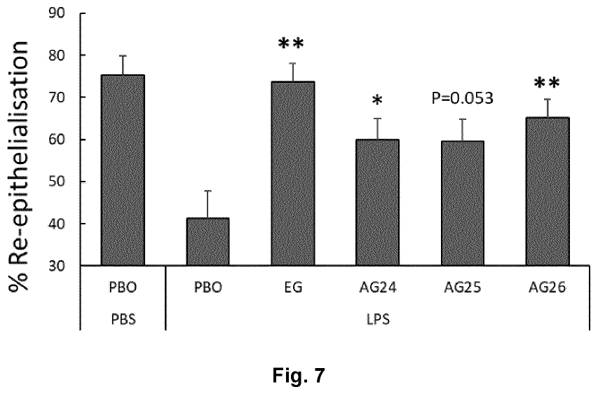

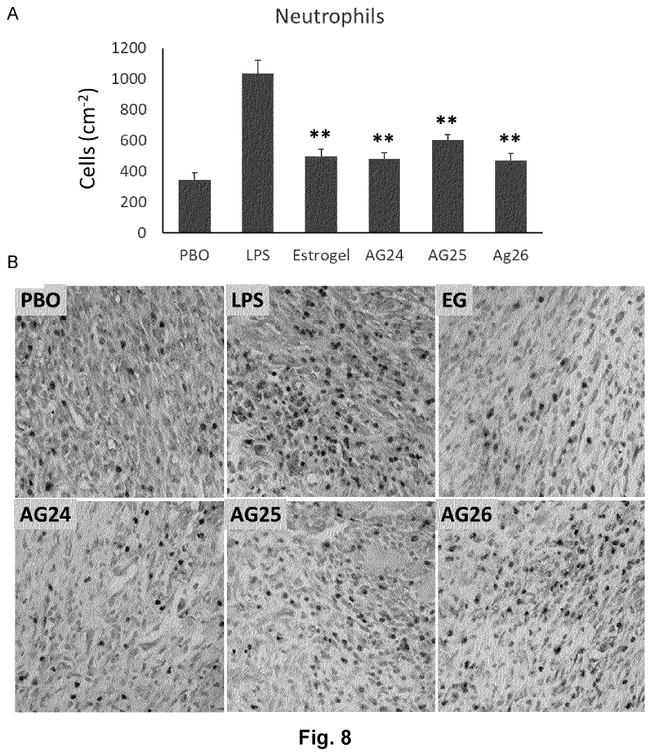

As expected. LPS and placebo treated wound displayed >30% delay in wound re-epithelialisation versus non-LPS treated wounds (FIG. 7). All four active treatments (EstroGel®, AG24, AG25 and AG26) promoted re-epithelialisation. Interestingly, a higher magnitude of promotion and statistical significance was observed for wounds treated with either EstroGel® or AG26, where re-epithelialisation approached that observed in the non-LPS treated control group. The AG25 treatment group just failed to reach statistical significance versus the LPS/PBO group.

Based on the data depicted on FIGS. 6 and 7, we conclude that the topically applied AG26 formulation can improve wound healing particularly well without having systemic effects such as uterine weight increase. This is indicative of the preferred dosage range if systemic effects are to be avoided completely. In some cases however, a higher dosage may be envisaged based on a favorable risk/benefit for the subject at hand.

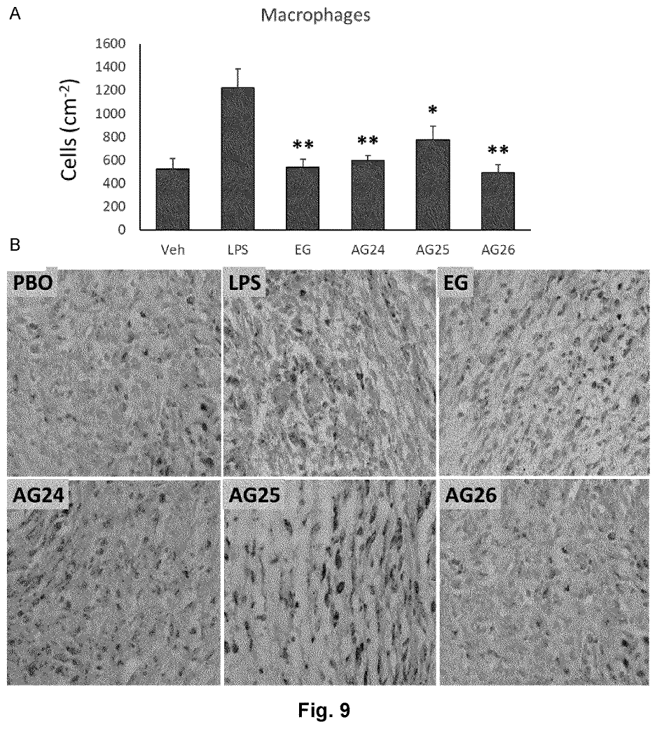

Immunohistochemistry was performed to evaluate the effect of active treatments (EstroGel®, AG24, AG25 and AG26) on local wound immune cell numbers. Wound neutrophil levels were substantially increased in LPS/PBO treated wounds versus non-LPS/PBO (FIG. 8). Treatment with AG24 and AG26 significantly reduced wound neutrophil numbers, to the same extent as EstroGel®, AG25 was marginally less effective at reducing wound inflammation, but still highly significant. Very similar effects of E2 and E4 containing gels were observed with respect to wound macrophage numbers (FIG. 9). Once again. AG25 was marginally less effective. Interestingly. EstroGel® and AG26 completely reversed the effect of LPS on macrophages, restoring wound macrophage levels equivalent to the non-LPS/PBO group (FIG. 9).

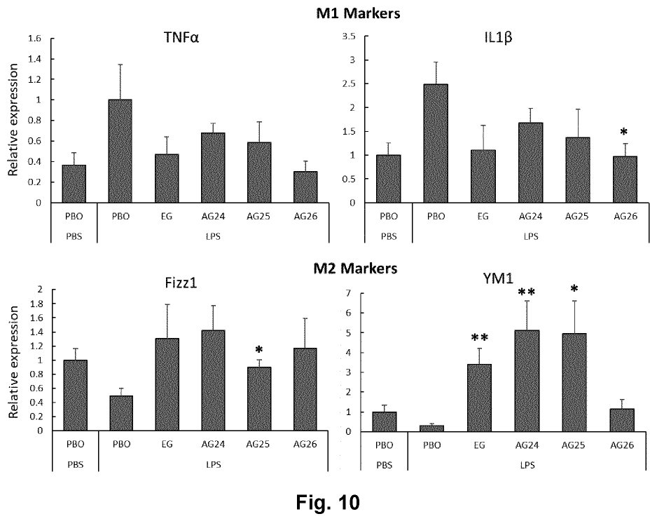

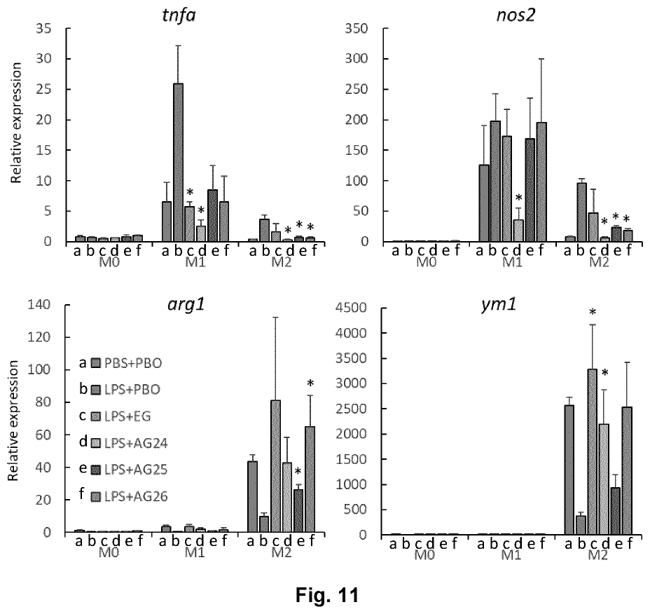

To further compliment the immunohistochemical analysis, wound tissue RNA was isolated and qPCR performed to evaluate specific markers of M1 and M2 polarisation phenotype (FIG. 10). All treatments led to a trend towards reduced M1 marker expression versus LPS/PBO control. Interestingly, the magnitude of effect appeared greatest in the Estrogel® and AG26 groups (reaching statistical significance for IL1-β and AG26 treatment). Conversely, the M2 markers Fizz1 and Yi1 were increased by all treatment groups, however, in this case the effect of AG26 was least pronounced of the three AG formulations tested. This data indicate that E4 both dampens inflammation and promotes a M2 (pro-healing) wound environment.

Finally, to further explore potential systemic effect of topical E2 or E4 treatment the phenotype of peritoneal macrophages isolated from each experimental mouse group upon study completion was assessed. Specifically, the macrophages from each group were separately isolated by peritoneal lavage, cultured and polarised towards either an M1 or M2 phenotype (as in example 7). RNA was isolated and qPCR was performed to quantify the relative levels of Tnf-α & iNOS (M1 markers), and Arg1 & Ym1 (M2 markers: FIG. 11).

It was found that macrophages isolated from the LPS/PBO group displayed increased M1 marker expression when polarised to M1 or M2 phenotype in vitro. Conversely, the LPS/PBO group displayed reduced M2 marker induction in response to M2 stimulation in vitro. Notably, the M0 LPS/PBO group did not display altered M1 or M2 marker expression versus control. To conclude, topical LPS primes peritoneal macrophages towards an exaggerated pro-inflammatory response.

In the LPS treated mice that had been topically treated with EstroGel®, or E4 formulations (AG24, AG25, AG26) these effects were reversed (FIG. 11). For example E4 formulation treatment completely reversed the LPS primed elevated expression of Tnf-α in both M1 and M2 in vitro polarised cells. A statistically significant reduction in expression was observed following treatment with AG24, AG25, and AG26. This anti-inflammatory effect was marginally greater with AG24 (the formulation with the highest E4 concentration), particularly when considered alongside iNOS expression (FIG. 11).

All four formulations increased the expression of the M2 markers. Arg1 and Ym1, in M2 polarised cells versus cells derived from the LPS/PBO treated mice (FIG. 11). This effect was most pronounced in macrophages isolated from the EstroGel® treated group, where the M2 marker levels in M2 polarised cells exceeded those from control (non-LPS treated) mice. Interestingly, of the three E4 formulations tested. AG26 (the lowest E4 concentration) delivered the highest increase in M2 marker expression, although AG25 and AG24 were also effective.

### Example 5. In Vivo LPS Treatment Protocol (Delaved Wound Healing Model): Comparison of Different Formulations and Variation of Treatment Duration

The in vivo LPS treatment protocol (delayed wound healing model) as presented in example 4 has been repeated with the following groups of animals:
Double placebo group (untreated control).
LPS control (LPS, placebo treatment).
LPS with topical EstroGel (estradiol gel, contains 0.06% 17β-estradiol as hemihydrate in a hydro-alcoholic gel), repeated administration for 4 days.
LPS with topical AG26 (E4 at 0.06%), single administration.
LPS with topical AG26 (E4 at 0.06%), repeated administration for 4 days.
LPS with topical AG28 (E4 at 0.06%), single administration.
LPS with topical AG28 (E4 at 0.06%), repeated administration for 4 days.

On day 0 only (single administration), or on days −1, 0, 1 and 2 (repeated administration), topical EstroGel. AG26. AG28 or corresponding placebo was applied in a thin layer over the top of the wounds (60 μl total: 30 μl/wound) during observations.

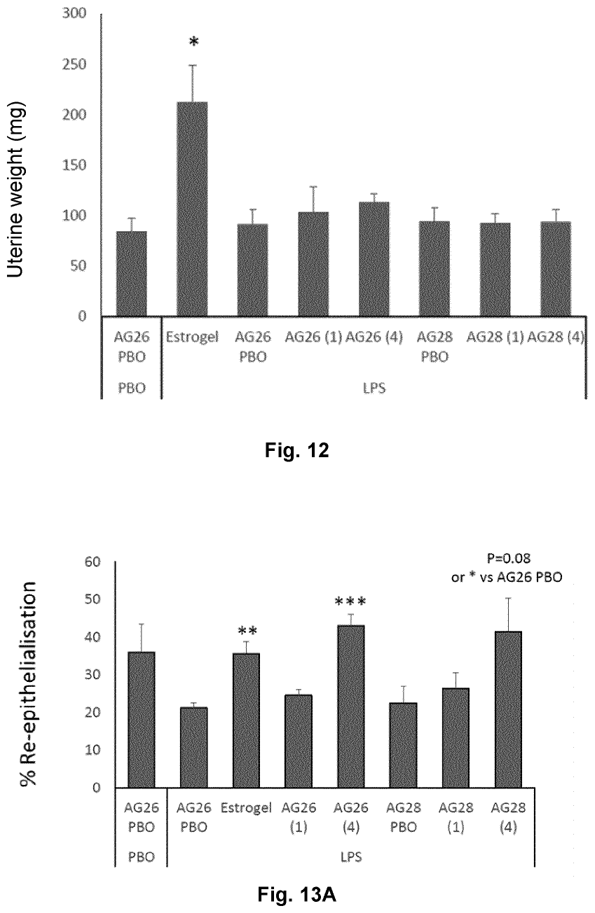

On day 3, mice were humanely culled via rising concentration of CO2 and cervical dislocation. Uteri as well as non-wounded and wound tissue samples were processed as described under example 4. In contrast to EstroGel, topical application of AG26 and AG28 did not affect uterine weight (FIG. 12).

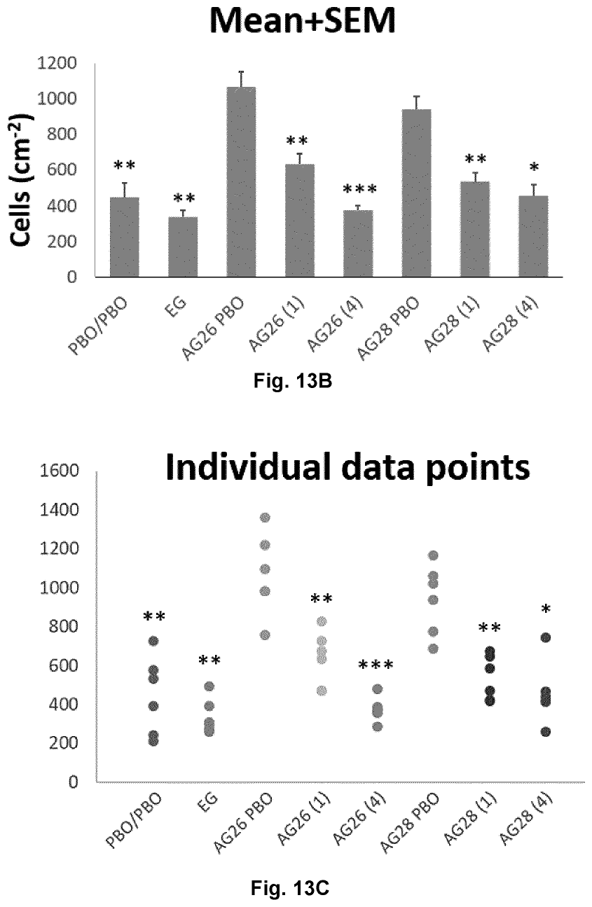

Next, we evaluated wound closure from histological samples (FIG. 13). Histological sections were subjected to K14 immunohistochemistry to visualise the newly forming epidermis. All three active treatments (EstroGel®. AG26 and AG28) promoted re-epithelialisation when administrated repeatedly for 4 days. AG28 just failed to reach statistical significance when compared to AG28 PBO group. Interestingly, a higher magnitude of promotion and statistical significance was observed for wounds treated with either AG26 compared to Estrogel®, where re-epithelialisation encompass that observed in the non-LPS treated control group.

Both AG26 and AG28 (4 applications) accelerated wound healing compared to the placebo by increasing re-epithelialisation in a delayed wound healing mouse model (FIG. 13).

AG26 and AG 28 are topical formulation that can improve wound healing after only one application without having systemic effects.

### Example 6. Wound Healing in db/db Mice

Female db/db 8 weeks old mice with diabetes will be weighed and animals will be split into six groups (6 animals per experimental group):
Topical placebo (control),
Topical EstroGel® (estradiol gel, contains 0.06% 17β-estradiol as hemihydrate in a hydro-alcoholic gel),
Topical AG24 (E4 at 0.5%),
Topical AG25 (E4 at 0.22%),
Topical AG26 (E4 at 0.06%),
Topical AG28 (E4 at 0.06%).

Mice will be anaesthetised using oxygen and isoflurane (flow rate 1.25-2 L with 2-2.5% isoflurane depending on clinical signs), shaved and dorsal skin will be cleaned with chlorhexidine wipes. Two 6 mm dorsal excisions will be created. EstroGel®, AG24, AG25, AG26, AG28 or placebo will then be applied in a thin layer over the top of the wound (60 μL total: 30 μL each wound). Buprenorphine (0.1 mg/Kg) will be administered post-operatively via subcutaneous injection in the scruff and each animal will be imaged. Mice will be recovered in a warming cabinet and returned to single housing. Observations will be performed post-operatively.

Every day, topical EstroGel®, AG24, AG25, AG26, AG28 or placebo will be re-applied in a thin layer over the top of the wounds (60 μL total: 30 μL each wound) during observations. Wound images will be performed on days 1, 3, 5, 7, 9, 11, 13 and 14 for planimetric analysis.

On day 14, mice will be sacrificed, and wound tissue will be bisected at their midpoint with the bottom half of each wound processed for wax histology (placed in a cassette with fixative). The uterus will be carefully removed to allow uterine weights to be documented.

EstroGel® application is expected to promote uterine weight gain and hypertrophic changes. Topical application of E4 is expected to affect uterine weight in a dose-dependent manner, with AG26 and AG28 showing no effect and AG24 inducing uterine weight gain.

EstroGel®, AG24, AG25, AG26 and AG28 is expected to accelerate wound healing compared to the placebo by increasing re-epithelialisation in a diabetic wound healing mice model.

We can conclude that AG26 and AG28 are topical formulations that can improve wound healing without having systemic effects.

### Example 7. Effects of Estetrol in Wound-Relevant In Vitro Assays

In vitro assays were performed to determine the effective dose of E4 in the wound relevant cell types (fibroblasts, keratinocytes and immune cells), informing E4 dose selection for clinical formulation studies. Second, a mechanistic understanding of the effects of E4 on wound-related cellular functions has been established.

### 1.0. Methods

### 1.1. Human and Murine Cells

Primary human dermal fibroblasts (HDF) were isolated from abdominal skin or leg skin. Primary neonatal normal human epidermal keratinocytes (NHEK) were purchased (Lonza). Murine dermal fibroblasts (MDF), murine epidermal keratinocytes (MEK), murine peritoneal macrophages and murine bone marrow were isolated from C57/B16 (wt). NDb (Lepr+/−) or Db (Lepr−/−) mice.

### 1.2. Culture of Dermal Fibroblasts and Epidermal Keratinocytes

Fibroblasts were isolated and cultured in DMEM supplemented with 10% heat inactivated FBS. 1% penicillin/streptomycin and 1% amphotericin B. At least 4 days prior to performing assays cells were switched to DMEM supplemented with 5% charcoal-stripped FBS.

Neonatal HEK were cultured in EpiLife supplemented with 15% human keratinocyte growth supplements (HKGS) and 1% penicillin/streptomycin. MEK were cultured in CnT basal medium supplemented with 1% penicillin/streptomycin and 1% amphotericin B.

### 1.3. Scratch Wound Assays

Cells were seeded into 24 well plates and cultured to form a confluent monolayer, and then scratched with a 1 ml pipette tip. Wells were treated with E2, E4 and/or various estrogen receptor agonists and antagonists. Stock solutions of E2, E4 and PHTPP were dissolved in ethanol (EtOH) so the final concentration of EtOH in the growth media (GM) did not exceed 0.1%. ICI, PHPTT, MPP, PPT and DPN were dissolved in DMSO so the final concentration of DMSO in the GM did not exceed 0.05%. A vehicle control containing the equivalent concentration of EtOH and/or DMSO was included, in addition to an untreated negative control. All treatments were diluted to working concentrations in media supplemented with 2% charcoal-stripped FBS. To visualise scratches at the defined endpoint, cells were stained with crystal violet and imaged on a Nikon E400 brightfield microscope. Scratch closure was determined from multiple independent measurements per well.

### 1.4. Culture of THP1 Cells

THP1 cells were maintained in RPM1 growth medium supplemented with 10% heat-inactivated FBS and 1% penicillin/streptomycin. Cells were seeded into 12- or 6-well plates at 2×10⁵ cells/ml and treated with phorbol 12-myristate 13-acetate (PMA) to induce differentiation to macrophages. Cells were cultured for 24 hours in complete growth medium without PMA, followed by a 6 hour serum starve prior to polarisation. Cells were polarised to M1 macrophages using 20 ng/ml IFN-γ and 10 pg/ml LPS (from E, coli) for 6 or 24 hours and collected for RNA isolation or flow cytometry.

### 1.5. Isolation and Culture of Murine Bone Marrow-Derived Macrophages

Bones were flushed with DMEM supplemented with 1% penicillin/streptomycin and 1% amphotericin B. Bone marrow cells were seeded into 12- or 6-well plates at 1×10⁶ cells/ml in DMEM supplemented with 10% FBS and 10% L929-conditioned medium to induce differentiation. After 7-10 days, differentiated macrophages were treated with 10⁻⁷M E2 or E4 in serum free growth medium for 16 hours and polarised to M1-type cells using 100 ng/ml IFN-γ and 1 μg/ml LPS. Following 6- or 24-hours polarisation cells were collected for RNA isolation or flow cytometry.

### 1.6. Isolation and Culture of Murine Peritoneal Macrophages

Peritoneal macrophages from C57/B16 mice were isolated by peritoneal lavage. The peritoneal cavity of euthanised mice were filled with 5 ml ice cold PBS supplemented with 3% FBS. Cell-containing lavage fluid was removed using a needle and syringe and cells seeded into 12- or 6-well plates at 1×10⁶ cells/ml in RPM1 growth medium supplemented with 10% charcoal-stripped FBS and 1% penicillin/streptomycin, were treated with 10-7M E2 or E4 in serum free growth medium for 16 hours and polarised to M1-type cells using 100 ng/ml IFN-γ and 1 μg/ml LPS. Following 6- or 24-hours polarisation cells were collected for RNA isolation or flow cytometry.

### 1.7. Isolation of RNA and Quantitative Real Time PCR

Following treatments, human and murine macrophages were collected in Trizol and RNA isolated using the Trizol plus RNA isolation kit (Invitrogen. Thermo Fisher) according to the manufacturer's instructions. RNA was reverse transcribed to cDNA using GoScript reverse transcriptase (Promega). Quantitative real-time PCR was carried out using 2×Takyon SYBR Green mastermix and a CFX Connect thermocycler. Primers for ERα and ERβ were used to explore changes in ER expression after treatment of MEK and MDF with E2 and E4. To investigate the effect of E2 and E4 on MEK differentiation and the expression of ECM proteins by MDF, primers for. Snail, keratin-1 and fibronectin, collagen I. MMP-2 and MMP-9 were used. Primers for INOS. IL-1B and Tnf-α or Ccl17 were used to assess M1/M2 polarisation status. Primers for GAPDH were used to normalise data. Relative gene expression was set to vehicle controls or M0 expression unless otherwise stated.

### 1.8 Statistical Analysis

Statistically significance was evaluated using one-way ANOVA with Tukey post hoc analysis or paired t-test as appropriate.

### 2.0. Keratinocytes and Fibroblasts

### 2.1. Both E2 and E4 Promote Human Dermal Fibroblast (HDF) Migration.

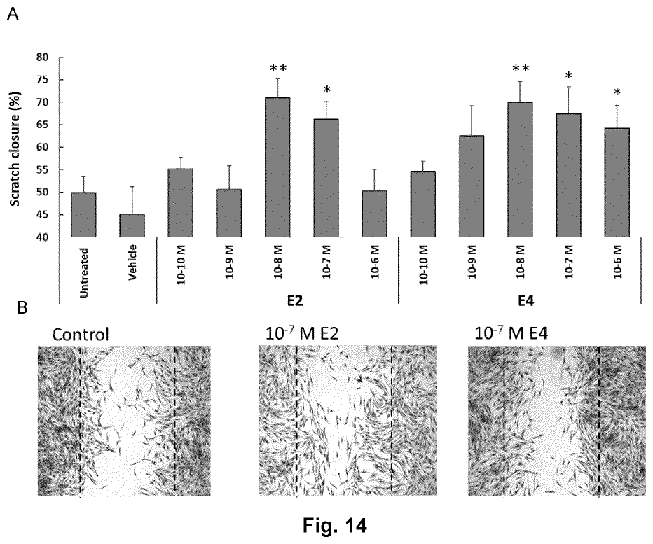

Scratch assays were performed to quantify the effect of E2 and E4 at varying concentrations on HDF migration (FIG. 14). Treatment with 10⁻⁸M and 10⁻⁷M E2 caused significantly faster scratch closure compared to vehicle control (157% and 141% respectively). At the highest concentration of E2 tested (10⁻⁶M) and at the two lowest concentrations 10⁻⁸M and 10⁻⁹M E2, no difference in closure was observed. E4 treatment also led to significantly faster scratch closure compared to vehicle control at 108M. 10⁻⁷M and 10⁻⁶M (152%. 147% and 142% respectively). Thus, both E2 and E4 optimally stimulated fibroblast scratch wound closure at 10⁻⁸M, with the higher magnitude of stimulation observed following E2 treatment.

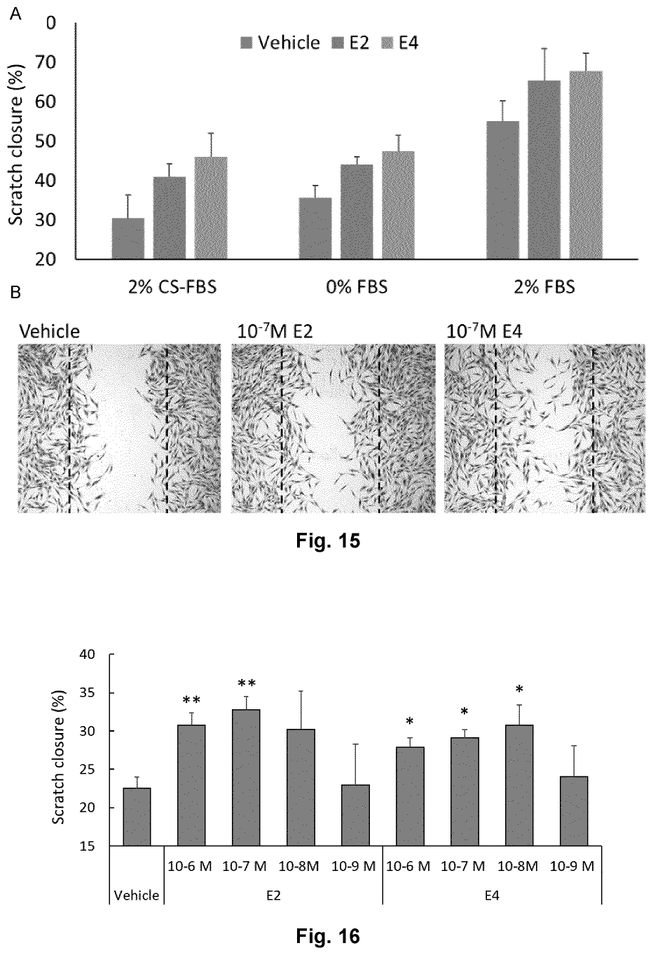

Next, an additional experiment was performed using HDF to a) further confirm the effects of E2 and E4 in fibroblasts from a fifth donor and b) explore the relative effect of including CS-FBS in the cell culture media. Three identical sets of scratch assays were performed (comparing vehicle to E2 or E4 treatment at 10⁻⁷M) in DMEM containing either 2% CS-FBS. 0% FBS or 2% FBS (not CS). Interestingly, in all three cases a trend was observed where both E2 and E4 promoted scratch closure, with the greatest promotion following E4 treatment (FIG. 15). As expected, the FBS (non-CS) group displayed faster closure across all treatments, however, the relative effect of E2 and E4 was less evident in this group.

### 2.2. Both E2 and E4 Promote Murine Dermal Fibroblast (MDF) Migration.

To further demonstrate the beneficial effects of E4, and to support dosing considerations for future in vivo mouse model studies, we next moved to perform in vitro scratch assays in murine dermal fibroblasts (MDFs). The biological effects of estrogenic compounds have been extensively demonstrated in murine models and switching to cells isolated from an inbred mouse strain was predicted to reduce model variability versus using cells isolated from human donors.

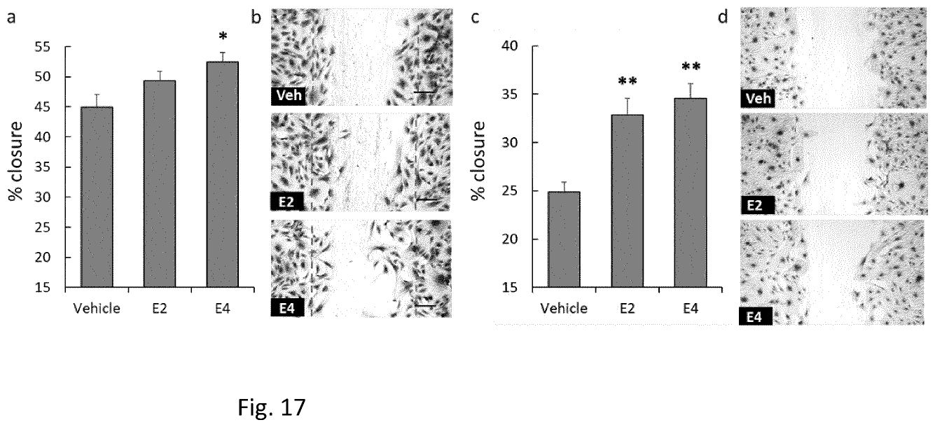

As in HDFs, treatment of MDFs with E2 or E4 significantly increased scratch closure compared to vehicle control treatment (FIGS. 16 & 17). In cells from a single mouse. E4 treatment magnitude of effect was less than E2 treatment, however the effective range appeared wider (significance at 3 concentrations for E4 vs 2 concentrations for E2: FIG. 16). Adding data from 2 additional mice and comparing low and high passage cells revealed slightly greater magnitude of effect for E4 versus E2 (FIG. 17). Moreover, the beneficial effects of both E2 and E4 were greatest in high passage cells, in line with clinical expectation.

2.3. E2 and E4 Treatment Increase Fibroblast ER Expression, while E4 Promotes Fibronectin Expression and Inhibits MMP Activity.

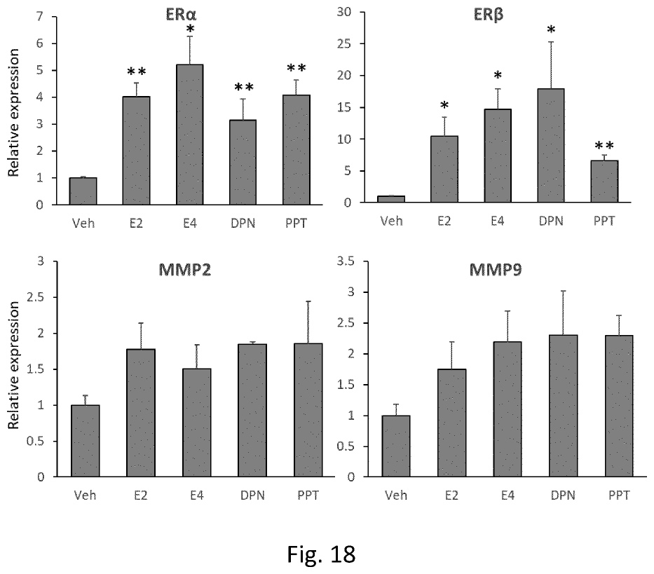

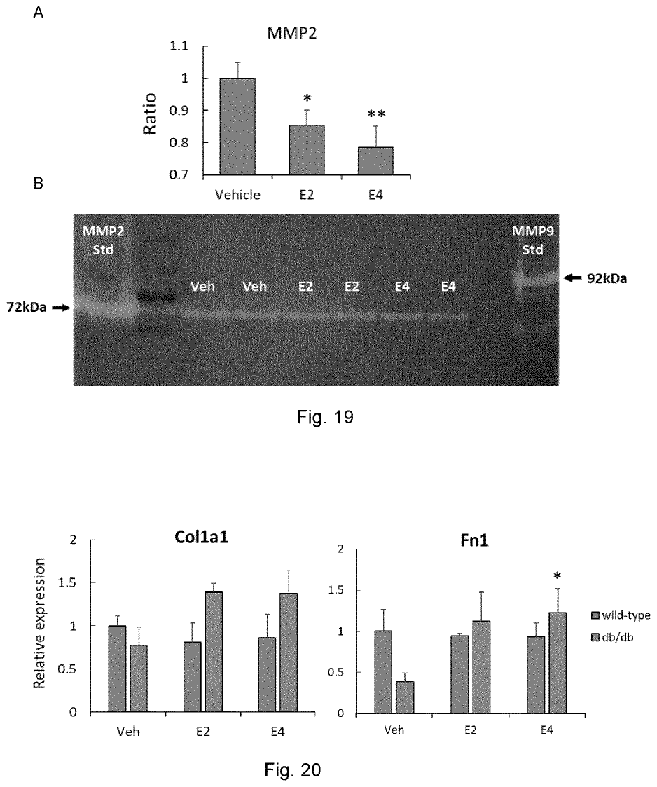

In vitro cultured murine fibroblasts were treated with either E2, E4, DPN or PPT at 10⁻⁷M. As previously reported. E2 treatment upregulated expression of both ERα and ERβ, while treatment with ER-agonists preferentially increased expression of their respective receptor (e.g. PPT increased ERα and DPN increased ERβ expression: FIG. 18). All treatments also showed a strong trend towards increased expression of MMP2 and MMP9 (FIG. 18). Next, zymography was performed on cell supernatants from HDFs treated with either E2 or E4, to evaluate the effect of E2 and E4 on cell-derived MMP activity. Across cells from 3 independent donors both E2 and E4 significantly reduced MMP2 activity versus control (FIG. 19). The magnitude of the effect (and statistical significance) was greater in cells treated with E4. No MMP9 activity was detected in any treatment group. Finally, we explored the effect of E2 and E4 on murine fibroblast expression of extracellular matrix genes, specifically collagen 1 (Colal) and fibronectin (Fn1) (FIG. 20). In cells derived from wild-type mice no effect was observed following treatment. However, when cells derived from diabetic (db/db) mice were treated with either E2 or E4 a trend towards increased expression of both Co1a1 and Fn1 was observed (FIG. 20). This increase in expression reached statistical significance for fibronectin (Fn1) in E4 treated db/db-derived cells alone.

### 2.4. E2 and E4 Promote Epidermal Keratinocyte Migration, and Modulate Wound-Relevant Epidermal Gene Expression

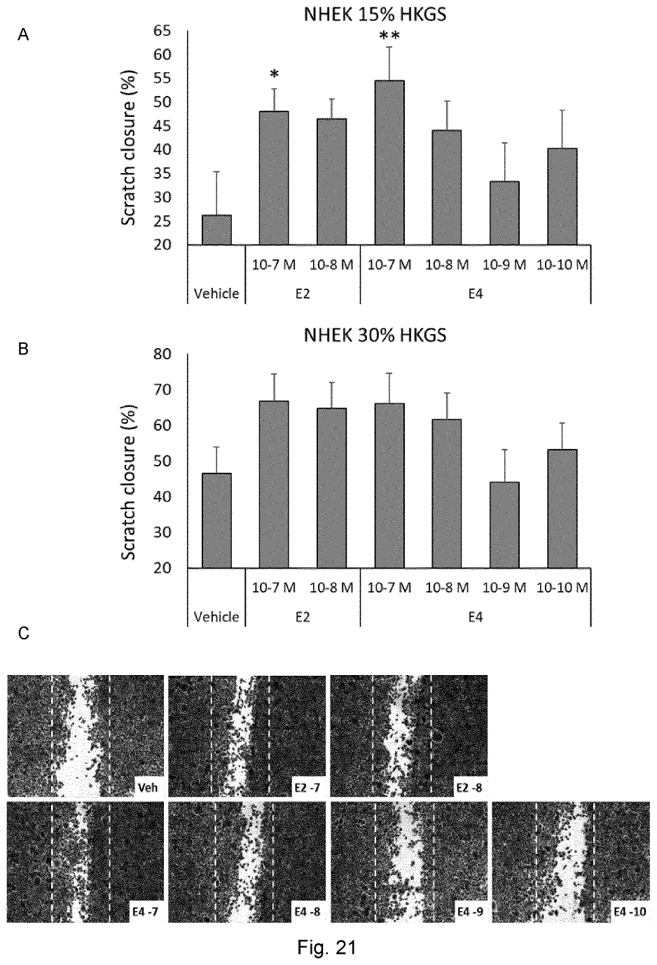

The effects of E2 and E4 on keratinocyte migration where evaluated using primary normal human epidermal keratinocytes (NHEKs). Experiments were performed in a range of grown media (with varying concentrations of human keratinocyte growth supplement (HKGS). Data shown for cells cultured in both 15% HKGS and 30% HKGS supplementation (FIG. 21). In both conditions E2 and E4 in the 10⁻⁷/10⁻⁸ M range displayed a strong trend to faster wound closure. Interestingly, this only reached statistical significance for 10⁻⁷M E2 and 10⁻⁷M E4 in 15% HKGS, with a slightly higher magnitude of effect and greater statistical significance noted for E4.

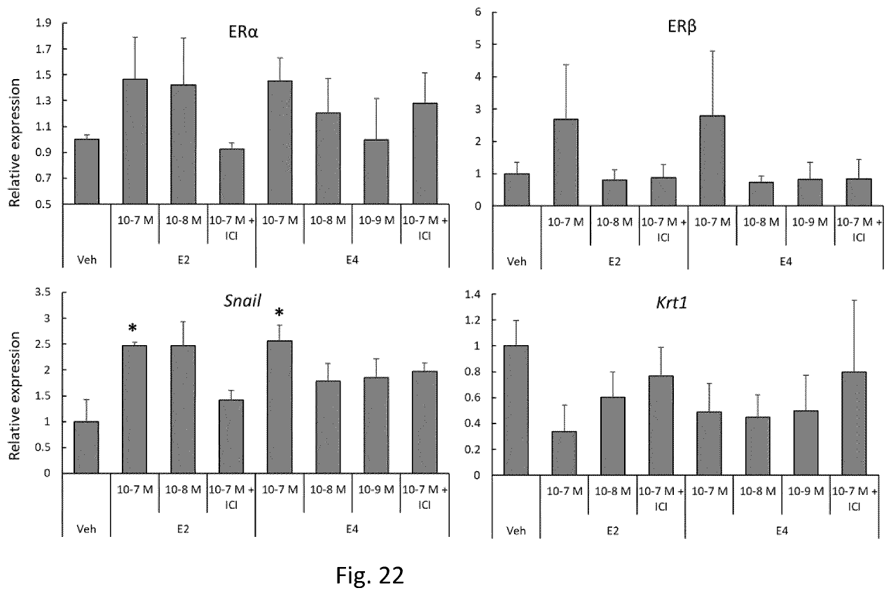

Next, we switched to primary mouse epidermal keratinocytes (MEKs) to explore the effect of E2 and E4 on MEK wound-relevant gene expression. Although not statistically significant, we observed a strong trend towards induction of both ERα and ERβ in cells treated with both E2 and E2 (particularly at 10⁻⁷M: FIG. 22). In line with the documented beneficial effects of E2 we observed statistically significant induction of the EMT marker. Snail, following treatment with either E2 or E4 at 10⁻⁷M. By contrast, the differentiation marker Keratin 1 (Krt1) displayed a trend towards downregulation with E2 and E4 treatment. Collectively, these data suggest a switch to a less differentiated pro-healing phenotype following treatment with either E2 or E4. In general, the observed effects of E2 and E4 on gene expression were reversed by co-treatment with the ER antagonist ICI.

### 2.5. E2 and E4 Display Anti-Inflammatory Activity In Vitro.

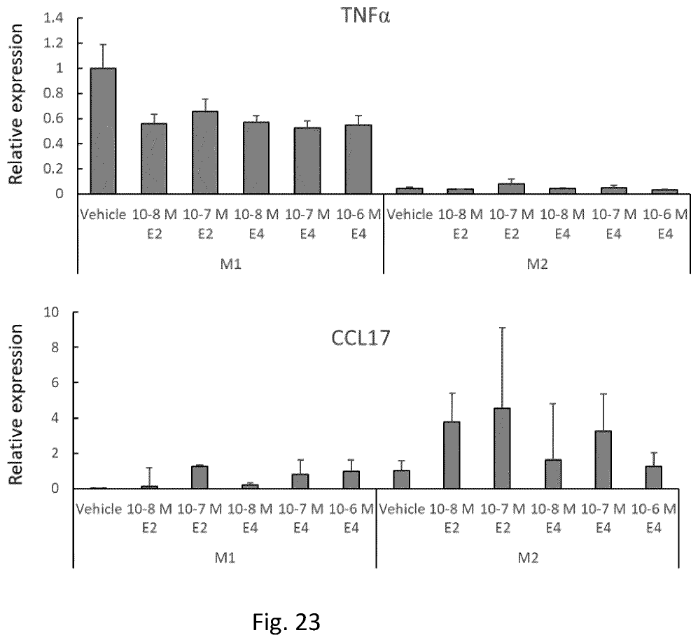

The relative anti-inflammatory effects of both E2 and E4 were evaluated in vitro. First, the human monocyte THP1 cell line was used to screen several concentrations of E2 and E4. THP1 cells were differentiated to a macrophage phenotype by treatment with PMA, followed by polarisation to an M1 or M2 phenotype (FIG. 23). Successful polarisation was confirmed by profiling the expression of the M1 marker (TNF-α) and the M2 marker (CCL17). The effects of co-treatment with a range of E2 or E4 concentrations were evaluated. Here, treatment with either E2 or E4 resulted in a strong trend towards reduced expression of Tnf-α in M1 polarised cells and increased expression of CCL 17 in M2 polarised cells (FIG. 23).

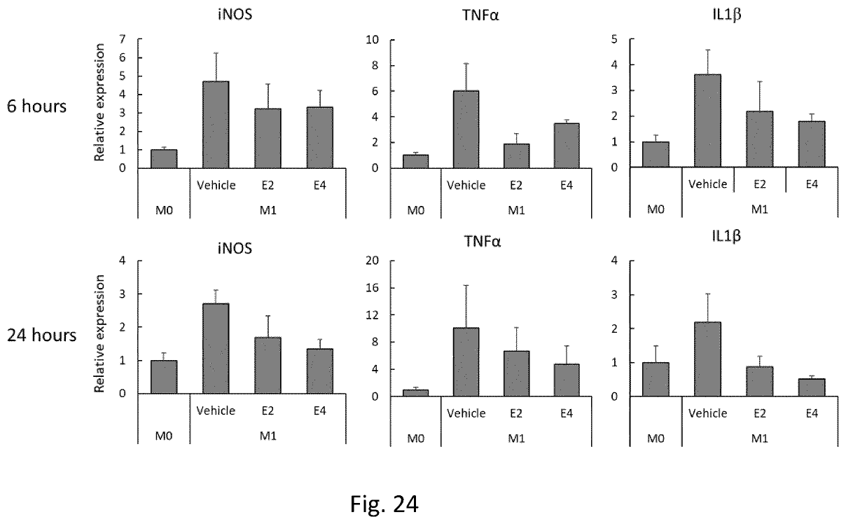

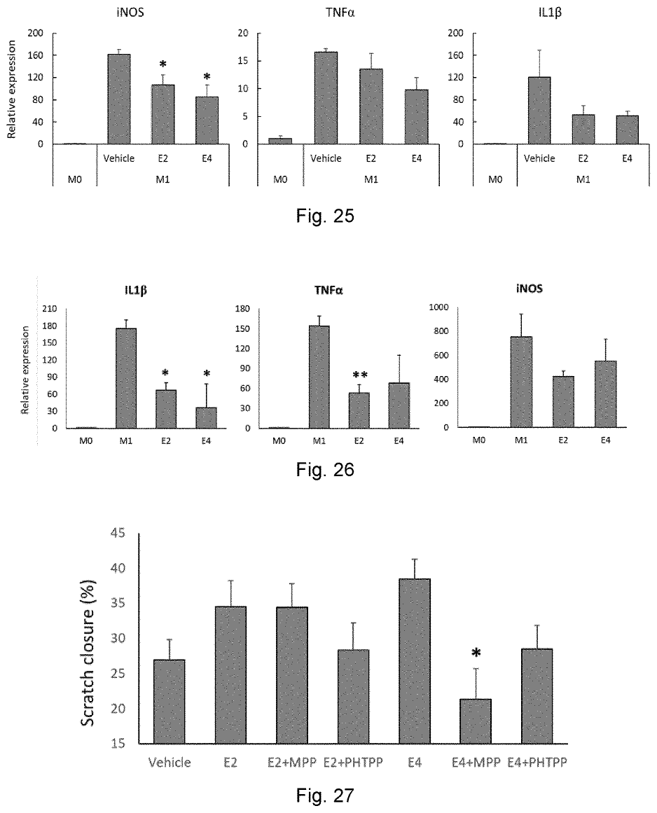

For follow up experiments murine bone marrow derived monocytes were isolated, differentiated (L929 media) and polarised to a pro-inflammatory M1 phenotype (IFN-γ and LPS: for 6 or 24 hours), and co-treated with either E2 or E4 at the optimal concentration identified in THP1 cells (10⁻⁷: FIG. 24). There was a trend toward decreased expression of a range of M1 markers (INOS, IL1-β and Tnf-α), in the presence of both E2 and E4, compared to treatment with vehicle. Similar effects were observed in cell polarised for either 6 hours or 24 hours (FIG. 24). Experiments were then undertaken with murine bone marrow derived monocytes differentiated to macrophages with 30 ng/ml MCSF, as opposed to L929 conditioned media. MCSF-stimulated macrophages displayed a far greater level of M1 marker expression, when polarised to M1 (versus M0). Again, a range of M1 markers (INOS, IL1- and Tnf-α) displayed a strong trend towards reduced expression the presence of both E2 and E4, compared to treatment with vehicle (FIG. 25). Note, both E2 and E4 led to a statistically significant reduction in the M1 marker INOS in MSCF-differentiated BMDMs. Generally, the observed anti-inflammatory effects of E4 were marginally greater than E2.

Finally, the anti-inflammatory effects E2 and E4 were evaluated in freshly isolated murine peritoneal macrophages. Unlike the BMDM protocol, the already differentiated peritoneal macrophages were immediately pre-treated with E2 (10⁻⁷M) or E4 (10⁻⁷M), followed by M1 polarisation using IFN-γ and LPS for 6 hours. In peritoneal macrophages a M1 markers (INOS, IL1-β and Tnf-α) also displayed a strong trend towards reduced expression the presence of both E2 and E4, compared to treatment with vehicle (FIG. 26). Note, the overall level of marker expression was higher in these cells. The E4-mediated reduction in IL1-β achieved statistical significance. The observed anti-inflammatory effects of E4 were broadly similar to those observed for E2.

### 2.6. Preliminary Evaluation of ER-Specific Effects on Fibroblasts and Immune Cells.

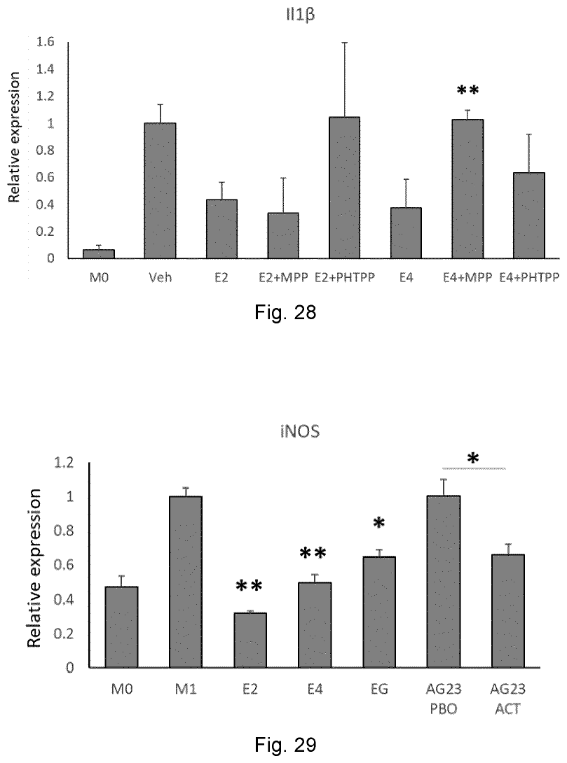

The relative ER-specific effects E4 were evaluated in vitro using co-treatment with highly specific antagonists for ERα (MPP) or ERβ (PHTPP). Preliminary studies suggest that the effects of E4 on both HDF migration (FIG. 27) and BMDM polarisation (FIG. 28) could be mediated by ERα. Specifically, the ERα antagonist MPP co-treatment appears to block E4-induced scratch closure and E4-mediated reduction in IL1-β, while the same ERα antagonist had limited influence on E2 effects. Given the relatively high variability these studies should be repeated with increase replicates to confirm the observed effects.

### 2.7. Preliminary Evaluation of the Activity of the Formulations In Vitro.

To provide initial confidence in the wound-healing potential of the formulated gels we performed in vitro evaluation of the AG23 active (ACT) and placebo (PBO) gel formulation, alongside EstroGel® (EG; FIG. 29). Once again. E2 and E4 treatment significantly dampened the M1 cell phenotype, as did treatment with E2-containing Estrogel®, AG23 placebo had no effect on relative iNOS expression, while AG23 ACT (containing E4) significantly reduced iNOS expression vs AG23 PBO. These data support further evaluation of E4-gel formulation in ex vivo human wounds.

### 3.0. Summary of Findings

Experiments were designed to explore the relative effects of E4 on defined cellular aspects of wound healing. A series of in vitro studies were undertaken using fibroblasts, keratinocytes and immune cells. Crucially, these in vitro studies were performed across both human and mouse cells, combining the clinical relevance of human cells with cross-species validation in murine cells to support follow on in vivo studies.

Fibroblasts. E4 was shown to promote the migration of both human (HDF) and mouse (MDF) dermal fibroblasts. In both cases the optimum concentration of E4 was between 10⁻⁷ and 10⁻⁸M, which was broadly similar to the optimum E2 concentration. Note, in each case the relative efficacy of E2 and E4 was also similar. In MDS we report that the effects on migration were greater in high passage cells (which may mimic the chronic wound environment. In MDFs treatment with either E2 or E4 directly increased cellular expression of ERα and ERβ. In HDSFs E4 treatment inhibited MMP2 activity in cell supernatants, and appeared to do so to a greater extent than E2. Preliminary evaluation suggests that ERα is important for mediating the effects of E4 on HDF migration.

Keratinocytes. Mouse epidermal keratinocytes (MEKs) also closed a scratch wound more quickly when treated with either E4 or E2. Again, the optimum concentration for both E2 and E4 was equivalent (10⁻⁷M), with the observed effects dependent on the composition of the cell growth media. As with fibroblasts, both E2 and E4 treatment induced MEK expression of ERα and ERβ (although the effect did not reach statistical significance). E2 and E4 treatment was shown to induce expression of the EMT marker Snail, and inhibit the differentiation marker keratin 1, both hallmarks of a cellular pro-healing response.

Anti-inflammatory activity. E4 and E2 were shown to exert pro-healing anti-inflammatory effects on mouse bone marrow derived macrophages (BMDM), mouse peritoneal macrophages and human THP-1 cells. In THP-1 cells E4 decreased expression of TNF-α (M1 marker) in M1 polarised cells and increased expression of CCL17 (M2 marker) in M2 polarised cells. In both murine BMDMs and peritoneal macrophages, E4 consistently reduced expression of a panel of M1 markers in M1 polarised cells. This effects was demonstrated at both 6 and 24 hours post-polarisation and using cells obtained with three independent methods of differentiation, L929 GM or MCSF (BMDM), or in vivo (peritoneal isolated macrophages). In each case the magnitude of effect observed with E4 broadly matched that observed using E2. Preliminary evaluation suggests that ERα is important for mediating the anti-inflammatory effects of E4 on BMDMs. Pilot studies revealed reduced M1 marker expression following treatment with the E4 formulation AG23.

### 4.0. References

Campbell L, Emmerson E, Davies F, et al. Estrogen promotes cutaneous wound healing via estrogen receptor beta independent of its antiinflammatory activities. J Exp Med. 2010: 207 (9): 1825-1833, doi: 10.1084/jem.20100500
Collaborative Group on Hormonal Factors in Breast Cancer. Type and timing of menopausal hormone therapy and breast cancer risk: individual participant meta-analysis of the worldwide epidemiological evidence. Lancet. 2019: 394 (10204): 1159-1168, doi: 10.1016/S0140-6736 (19) 31709-X
Roth G S, Harman S M, Lamberg S I. Altered Ovarian Regulation of Wound Healing during Aging. Proceedings of the Society for Experimental Biology and Medicine. 1981: 166 (1): 17-23, doi: 10.3181/00379727-166-41017
Thornton M J. Estrogens and aging skin. Dermatoendocrinol. 2013: 5 (2): 264-270, doi: 10.4161/derm.23872

### Example 8. Preparation of Aqueous Gel Formulations for Stability Studies

Table 8 lists the hydrogels, that were prepared for stability studies. Preparation took place as described for AG24 in Example 1.

TABLE 8
Compositions (% w/w) of estetrol (E4) aqueous gel
formulations prepared for stability studies.

| Formulation | 1 | 2 | 3 | 4 | 5 |
|---|---|---|---|---|---|
| Estetrol (E4) | 0.06 | 0.40 | 0.50 | 0.06 | 0.50 |
| PEG 400 | 43.95 | 43.95 | 43.95 | 43.95 | 43.95 |
| Transcutol ® P | 1.00 | 1.00 | 1.00 | 2.50 | 2.50 |
| Water (1st Addition) | 48 | 48 | 48 | 48 | 48 |
| Carbopol ® 980 | 0.50 | 0.50 | 0.50 | 0.50 | 0.50 |

| | |
|---|---|
| Sodium hydroxide | Adjust to pH 6-6.5 |
| Water (2^{nd} addition) | q.s. 100% |
| TOTAL | 100 |

1. A composition for topical application, comprising of from about 0.01% to about 5%, preferably of from 0.02% to 1.5% (w/w) of an estetrol component, or of from about 0.02% to about 1% (w/w) of an estetrol component, or of from about 0.05% to about 1.2% (w/w) of an estetrol component, more preferably of from about 0.09% to about 1.1% (w/w), even more preferably from 0.1% to 1% (w/w) of an estetrol component, most preferably of from 0.3% to 0.7% (w/w) of an estetrol component.

2. The composition according to claim 1, further comprising a permeation enhancer that enables permeation through the stratum corneum of the skin of a subject.

3. A hydrogel formulation comprising the composition according to claim 1 or 2.

4. The composition or hydrogel according to any one of claims 1 to 3, for use in topical wound healing.

5. The composition, composition for use, hydrogel, or hydrogel for use according to any one of claims 1 to 4, which is a formulation selected from the group consisting of: emulsions, suspensions, ointments, pastes, lotions, gels, foams, mousses, and creams.

6. The composition, composition for use, hydrogel, or hydrogel for use according to any one of claims 1 to 5, comprising a permeation enhancer in an amount of from 0.05% to 60% (w/w), preferably in an amount of from 0.1% to 5% (w/w).

7. The composition, composition for use, hydrogel, or hydrogel for use according to claim 6, wherein said permeation enhancer comprises a substance or molecule that enables permeation through the stratum corneum and a solvent, preferably wherein said permeation enhancing molecule is selected from the group consisting of: ethanol, ether such as diethylene glycol monoethyl ether (Transcutol®), benzyl alcohol, fatty acids and esters thereof, or any combination thereof: or
wherein said permeation enhancer comprises a solvent comprising one or more polyethylene glycols (PEG), propylene glycol (PG), or combinations thereof.

8. The composition, composition for use, hydrogel, or hydrogel for use according to claim 7, wherein said PEG is a PEG having a molecular weight of between about 200 g/mol and about 600 g/mol such as a PEG selected from the group consisting of: PEG200, PEG300, PEG400, PEG500, PEG600, or any combination thereof.

9. The composition, composition for use, hydrogel, or hydrogel for use according to any one of claims 1 to 8, further comprising benzyl alcohol, preferably in an amount of from 1% to 3% and/or further comprising a thickener in an amount of from 0.3% to 20% (w/w), preferably comprising a thickener in an amount of from 0.5% to 3% (w/w).

10. The composition, composition for use, hydrogel, or hydrogel for use according to any one of claims 1 to 9, wherein said thickener is selected from the group consisting of a hydroxyethyl cellulose (HEC), a carboxymethyl cellulose (CMC), a high molecular weight cross-linked acrylic based polymer, a non-ionic triblock copolymer, or any combination thereof, preferably wherein said high molecular weight cross-linked acrylic based polymer is Carbopol®, or wherein said HEC is HEC250 HHX, or wherein said non-ionic triblock copolymer has an approximate molecular mass of between about 1800 and about 4000 and a polyoxyethylene content of from about 70 to about 80%.

11. The composition, composition for use, hydrogel, or hydrogel for use according to any one of claims 1 to 10, further comprising a preservative in an amount of from 1% to 10% (w/w), preferably of from 1% to 3% (w/w), preferably wherein said preservative is selected from the group consisting of: lysozyme, nisin, quaternary ammonium preservatives, parabens, phenoxyethanol, benzyl alcohol, chlorobutanol, phenol, sorbic acid, thimerosal, natural preservatives, and any combination thereof.

12. The composition, composition for use, hydrogel, or hydrogel for use according to any one of claims 1 to 11, further comprising an emollient in an amount of from 2.5% to 30% (w/w), preferably of from 8% to 12% (w/w), preferably wherein said emollient is selected from the group consisting of: glycerol, acetyl alcohol, stearyl alcohol, stearic acid, isopropyl palmitate, squalene, lanolin, glycerin, petrolatum, petroleum, and any combination thereof.

13. The composition, composition for use, hydrogel, or hydrogel for use according to any one of claims 1 to 12, wherein said composition or formulation is complemented to 100% (w/w) by means of an aqueous solution such as water.

14. The composition, composition for use, hydrogel, or hydrogel for use according to any one of claims 1 to 4, comprising:
from about 0.1% to about 5% (w/w) of a permeation enhancer, preferably from about 1% to about 2.5% (w/w/) of a permeation enhancer, more preferably wherein said permeation enhancer comprises a permeation enhancer molecule and a solvent or solvent system;
from about 0.3% to about 3% (w/w) of a thickener;
optionally a preservative and/or an emollient;
water up to 100% (w/w); or
or comprising:
from about 38% to about 45% (w/w) PEG400;
from about 0.1% to about 1% (w/w) Carbopol®; and
from about 0.8% to about 3% (w/w) Transcutol®.

15. The composition, composition for use, hydrogel, or hydrogel for use according to any one of claims 1 to 14, for use in treatment of acute wounds, surgical wounds, wounds caused by acute injury, chronic wounds, wounds caused and/or maintained by a diabetic disease, wounds of subjects having impaired wound healing including an impaired delayed cutaneous wound healing or bacterially delayed wound healing, wound healing characterized by reduced wound edge migration, infected wound sites, combat wounds, burns, and chronic leg ulcers.

16. The composition, composition for use, hydrogel, or hydrogel for use according to any one of claims 1 to 14, comprised in a wound dressing, bandage, band-aid, patch, or plaster.

17. A packaging comprising one or more dosage units of the composition hydrogel according to any one of claims 1 to 14, preferably wherein said packaging unit is a box, a display unit, an ampoule, a bottle, a vial, a tube, a syringe, a cartridge, a bag, a sachet, a pouch, a film, a laminate, a foil, a can, a cylinder, or a pressurized container.
